(19) United States
(12) Patent Application Publication
(10) Pub. No.: US 2017/0266269 A1
(43) Pub. Date: Sep. 21, 2017

(54) METHODS OF IDENTIFYING ANTIGENS FOR VACCINES

(71) Applicant: UNIVERSITY OF WASHINGTON THROUGH ITS CENTER FOR COMMERCIALIZATION, Seattle, WA (US)

(72) Inventors: Mary L. DISIS, Renton, WA (US); Denise CECIL, Shoreline, WA (US); Meredith SLOTA, Seattle, WA (US)

(21) Appl. No.: 15/300,257

(22) PCT Filed: Mar. 27, 2015
(86) PCT No.: PCT/US15/23149

§ 371 Date: Sep. 28, 2016

Related U.S. Application Data
(60) Provisional application No. 61/972,179, filed on Mar. 28, 2014.

(51) Int. Cl. A61K 39/00; A61K 48/00; G01N 33/68

(52) U.S. Cl. CPC A61K 39/0011; G01N 33/6878; G01N 33/6866; G01N 33/6869; A61K 48/0091; G01N 2333/70539; G01N 2333/57; G01N 2333/5428; A61K 2039/53; A61K 2039/57; A61K 2039/70

(57) ABSTRACT

The methods, processes, and systems described herein include identifying an epitope of a peptide that may elicit an immune response in a subject. Often the methods, systems and processes may include designing and producing a composition comprising an epitope of a peptide identified using the methods or processes described herein.

### CROSS-REFERENCE

This application claims the benefit of U.S. provisional patent application No. 61/972,179, filed Mar. 28, 2014, which is herein incorporated by reference in its entirety.

### STATEMENT AS TO FEDERALLY SPONSORED RESEARCH

This invention was made with the support of the United States government under grant number W81XWH-11-1-0760 by the Department of Defense.

### BACKGROUND

Vaccines preventing the onset or progression of diseases have historically advanced slowly from clinical testing into standard of care use despite decades of development and evaluation. The majority of vaccines that have advanced to later stage clinical trials have been peptide- or tumor cell-based. Both peptide and tumor cell-based vaccine platforms supply intact antigen for presentation to the immune system. In general, results of clinical trials using each type of vaccine has shown little or modest clinical efficacy in definitive randomized trials. Obstacles to improved efficacy have included the poor immunogenicity of self-peptides which are disease-associated antigens, low to moderate immune responses to the vaccination, and the observation that active immunization against self-antigens can induce immune suppressive cells, such as T-regulatory cells (Treg), to proliferate. New methods for identifying disease-associated antigens which elicit desired immune responses are needed.

### SUMMARY

The methods described herein include a method for designing a plasmid vaccine, the method comprising: determining a potential of a set of putative epitopes to induce a sub-type of an immune response; ranking a plurality of putative epitopes from the set of putative epitopes by the sub-type of the immune response; from the plurality of ranked putative epitopes, identifying a set of desired epitopes such that the set of desired epitopes induces a desired sub-type of an immune response in a subject; and arranging the desired epitopes to provide a plasmid vaccine design. The methods described herein further include a method for designing a peptide vaccine, the method comprising: determining a potential of a set of putative epitopes to induce a sub-type of an immune response; ranking a plurality of putative epitopes from the set of putative epitopes by the sub-type of the immune response; from the plurality of putative epitopes ranked in step (b), identifying a set of desired epitopes such that the set of desired epitopes induces a desired sub-type of an immune response in a subject; and arranging the desired epitopes to provide a plasmid vaccine design.

Also described herein include systems and processes for designing a plasmid vaccine and/or for designing a peptide vaccine.

The systems described herein include a system for designing a plasmid vaccine, which comprises a digital processing device comprising an operating system configured to perform executable instructions, and an electronic memory; a set of putative epitopes stored in the electronic memory; a computer program including instructions executable by the computer to create an application comprising: (i) a first software module configured to determine the potential of each putative epitope within the set of putative epitopes to induce a sub-type of an immune response; (ii) a second software module configured to rank a plurality of putative epitopes from the set of putative epitopes by the sub-type of the immune response, and identify a set of desired epitopes from the ranking, wherein the set of desired epitopes is capable of inducing a desired sub-type of an immune response in a subject; and (iii) a third software module configured to design a plasmid vaccine from the set of desired epitope identified in step (ii).

The systems describes herein further include a system for designing a peptide vaccine, which comprises a digital processing device comprising an operating system configured to perform executable instructions, and an electronic memory; a set of putative epitopes stored in the electronic memory; a computer program including instructions executable by the computer to create an application comprising: (i) a first software module configured to determine the potential of each putative epitope within the set of putative epitopes to induce a sub-type of an immune response; (ii) a second software module configured to rank a plurality of putative epitopes from the set of putative epitopes by the sub-type of the immune response, and identify a set of desired epitopes from the ranking such that the set of desired epitopes is capable of inducing a desired sub-type of an immune response in a subject; and (iii) a third software module configured to design a peptide vaccine from the set of desired epitope identified in step (ii).

The processes described herein include a plasmid vaccine designed by the process of determining the potential of each putative epitope within the set of putative epitopes to induce a sub-type of an immune response; ranking a plurality of putative epitopes from the set of putative epitopes by the sub-type of the immune response; identifying a set of desired epitopes from the ranking such that the set of desired epitopes is capable of inducing a desired sub-type of an immune response in a subject; and designing a plasmid vaccine from the set of desired epitopes.

The processes described herein further include a peptide vaccine designed by the process of determining the potential of each putative epitope within the set of putative epitopes to induce a sub-type of an immune response; ranking a plurality of putative epitopes from the set of putative epitopes by the sub-type of the immune response; identifying a set of desired epitopes from the ranking such that the set of desired epitopes is capable of inducing a desired sub-type of an immune response in a subject; and designing a peptide vaccine from the set of desired epitopes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a flow diagram for epitope identification.

FIG. 2 shows a heat map of Th epitope prediction.

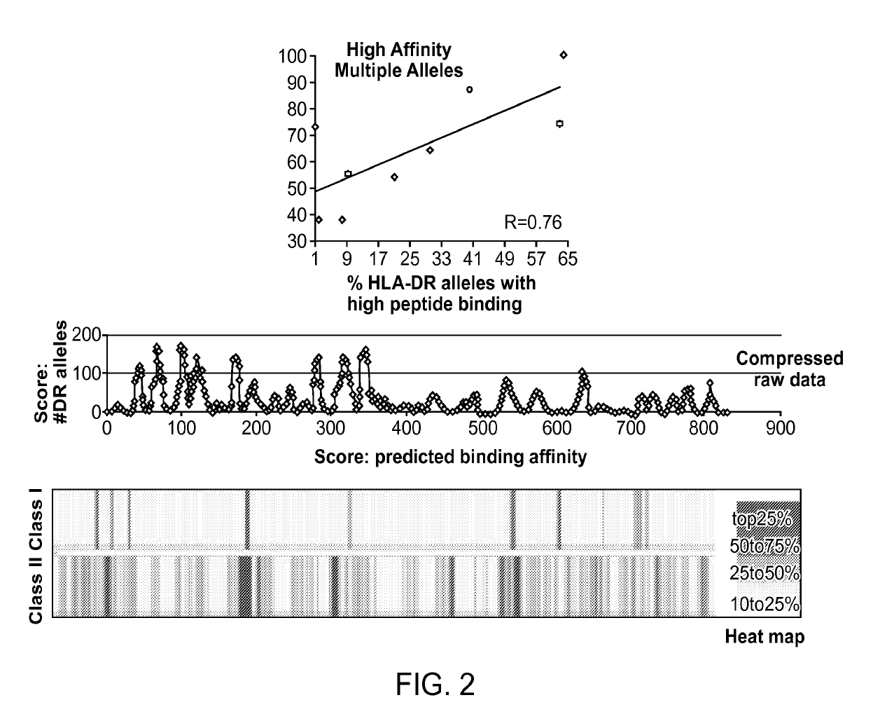

FIG. 3 shows a list of the candidate stem cell/EMT peptides.

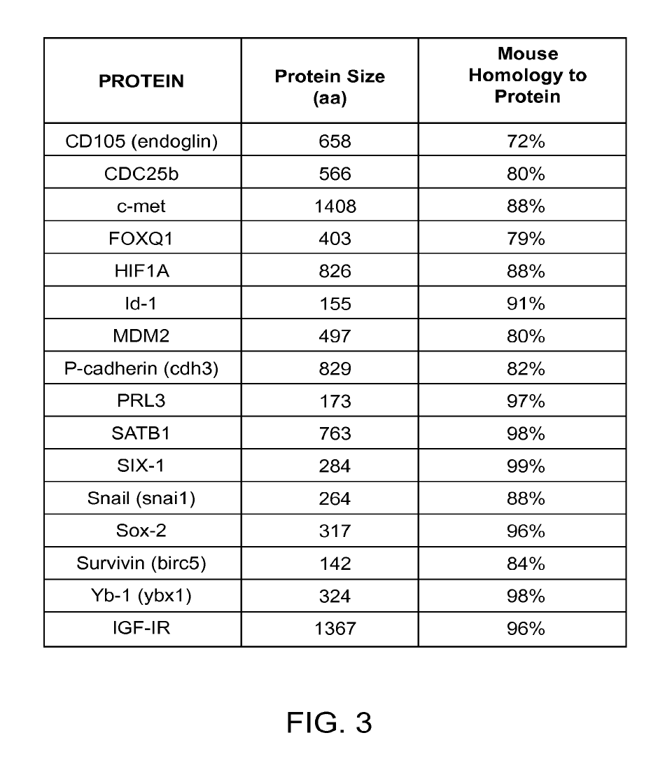

FIG. 4 shows a flow diagram of selection process for candidate stem cell/EMT peptides.

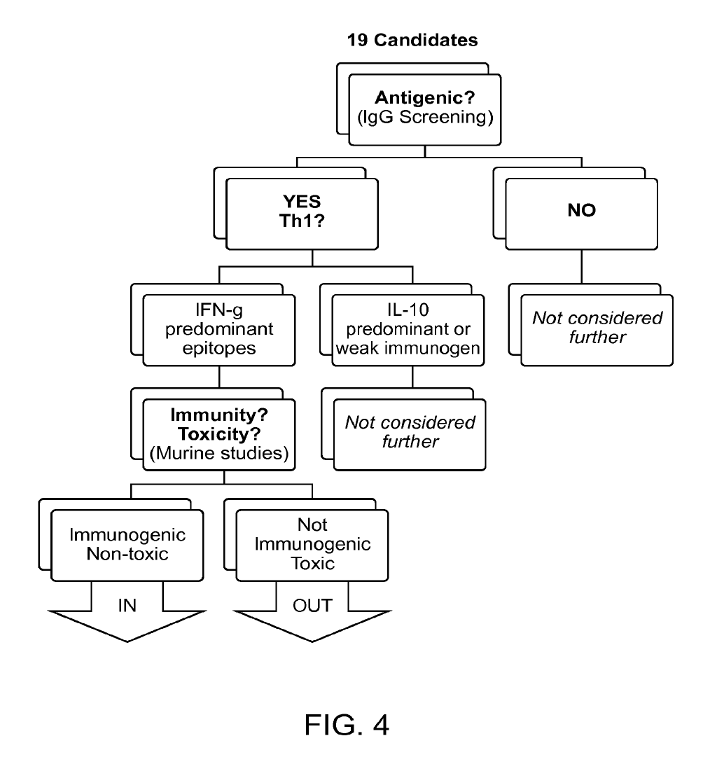

FIG. 5 shows a list of stem cell antigen homologies across selected species.

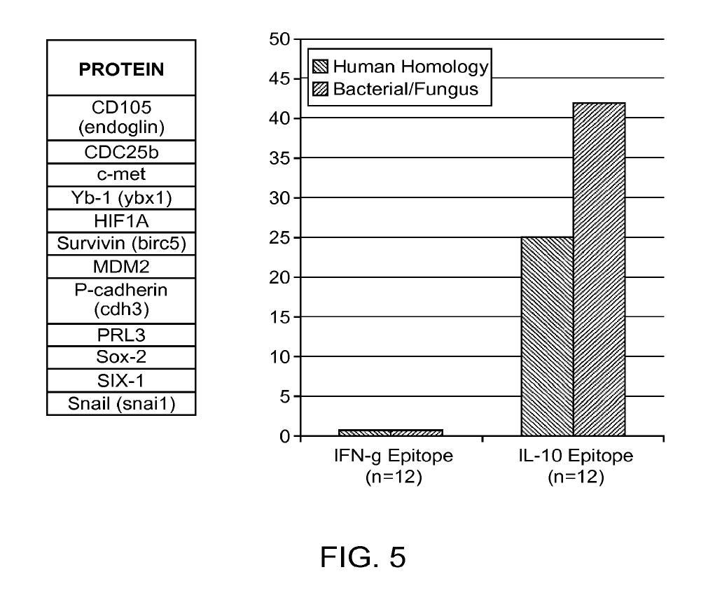

FIG. 6 shows a graph of the in silico multi-score of the CD105 extended epitope.

FIG. 7 shows a graph of the in silico multi-score of the HIF1α extended epitope.

FIG. 8 shows that the in vitro peptide binding affinity correlates with in vivo immunogenicity.

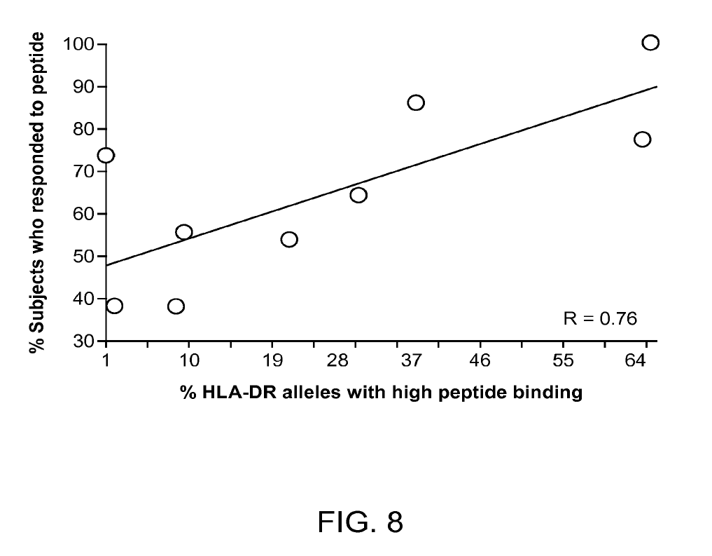

FIG. 9 shows that the IGFBP-2 epitope-specific Th2 higher functional avidity and homology to a greater number of bacterial and self-peptides than IGFBP-2 epitope-specific Th1.

FIG. 10 shows an exemplary diagram of a composition derived from the methods described herein. The sequence of the STEMVAC fusion protein illustrated in FIG. 10 is referenced as SEQ ID NO: 15.

FIG. 11 shows that an N-terminus, but not C-terminus, IGFBP-2 vaccine both stimulates Type I immunity and inhibits tumor growth.

FIG. 12 depicts an IGFBP-2 vaccine-induced Th2 immune response abrogates the anti-tumor effect of IGFBP-2-specific Th1.

FIG. 13 shows exemplary cytokine secretion patterns induced by HER2 vaccination.

FIG. 14 shows IGF-1R epitopes screened for IFNγ and IL-10 T-cell secretion by ELISPOT.

FIG. 15 demonstrates that the IGFBP-2 C-terminus is enriched for epitopes that induce IL-10-secreting T-cells as compared to the N-terminus.

FIG. 16 shows extended epitopes for Yb-1 based on IFN/IL-10 activity ratio.

FIG. 17 shows magnitude and incidence of IFNγ predominant. IFNγ/IL-10 activity ratios for the CDH3 antigen.

FIG. 18 shows magnitude and incidence of IFNγ predominant. IFNγ/IL-10 activity ratios for the HIF1α antigen.

FIG. 19 shows magnitude and incidence of IFNγ predominant. IFNγ/IL-10 activity ratios for the CD105 antigen.

FIG. 20 shows magnitude and incidence of IFNγ predominant. IFNγ/IL-10 activity ratios for the MDM-2 antigen.

FIG. 21 shows magnitude and incidence of IFNγ predominant. IFNγ/IL-10 activity ratios for the SOX-2 antigen.

FIG. 22 shows that Th2 abrogates the anti-tumor efficacy of Th1.

FIG. 23 depicts HIF1α peptide and plasmid vaccine immunogenicity and efficacy in mice.

FIG. 24 depicts CD105 peptide and plasmid vaccine immunogenicity and efficacy in mice.

FIG. 25 depicts CDH3 peptide and plasmid vaccine immunogenicity and efficacy in mice.

FIG. 26 depicts SOX2 peptide and plasmid vaccine immunogenicity and efficacy in mice.

FIG. 27 depicts MDM2 peptide and plasmid vaccine immunogenicity and efficacy in mice.

FIG. 28 shows the mass of mice three months after the last vaccine.

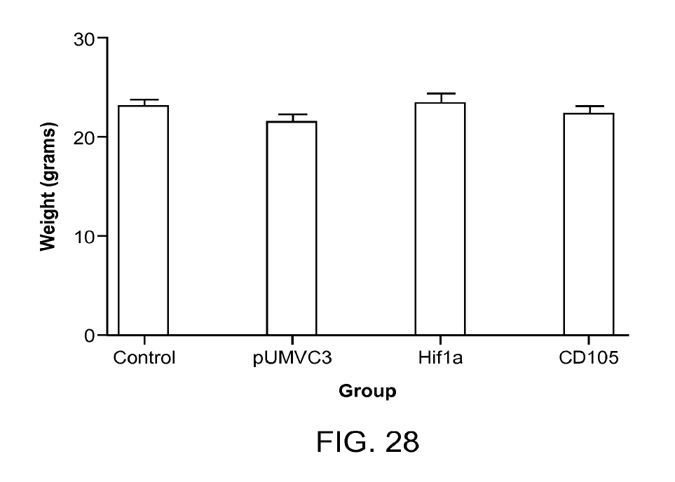

### DETAILED DESCRIPTION

As described in greater detail herein, the methods, systems, and processes of the disclosure include the identification of one or more epitopes from one or more peptides, often a specific set of self-peptides. In some cases, the amino acid sequence of the identified one or more epitopes may be incorporated into a composition, often a vaccine. In other cases, the one or more nucleotide sequences encoding the amino acids of the one or more epitopes may be incorporated into at least one plasmid, the at least one plasmid thereby incorporated into a composition, often a plasmid-based vaccine. In other cases, amino acids of the one or more epitopes may be incorporated into a composition, often a peptide-based vaccine. The one or more epitopes identified using the methods described herein may thereby be antigenic when compounded into a composition and administered to a subject. Often, administration of the composition to the subject may provide a desired set of benefits to a subject in need thereof.

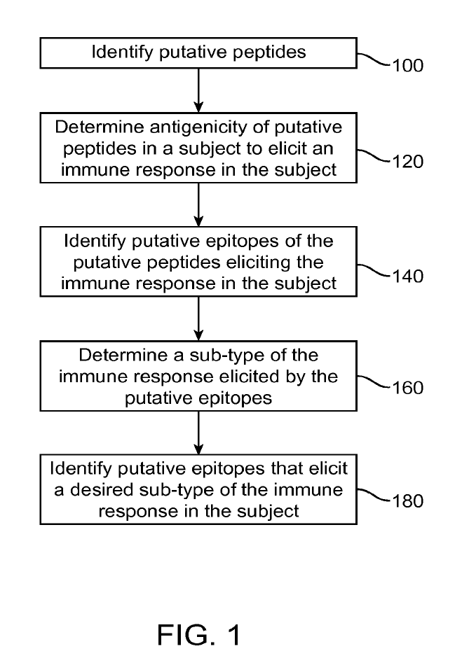

The methods, systems, and processes may comprise identifying one or more putative peptides, often a set of peptides, for example self-peptides (e.g., about 2 to about 50 different peptides), the expression of the one or more peptides deregulated in a subject that may have or may develop a disease. (FIG. 1, 100) In some cases, the disease may be a prognosis, a pathophysiological condition or homeostatic state. For example, the disease may include, but is not limited to cancer, autoimmune disease and metabolic disease. In some cases, the one or more putative peptides may be identified. For example, identification methods or processes may include, performing at least one of the following. a literature search, a database search, a search of bioinformatics mediums, an analysis of a fluid sample from a subject, an analysis of a cellular sample from a subject or an analysis of a tissue sample from a subject. In some cases, the sample from a subject may be blood, other body fluids, tissue, cells or the like. In some cases, the sample may be isolated from a subject with a disease (e.g., a patient) and/or control subject (e.g., a subject without a disease).

The methods, systems, and processes may further comprise determining the antigenicity of one or more identified putative peptides in a subject, often a human subject. (FIG. 1, 120). In some cases, the one or more identified putative peptides are self-peptides. In some cases, the antigenicity may be determined by the detection of an immune response elicited by a subject following administration of nucleic acids encoding the one or more putative identified peptides to the subject. In some cases, the antigenicity may be determined by the detection of an immune response elicited by a subject following administration of the one or more putative identified peptides to the subject. For example, the immune response may be detected by determining the activity of immune cells, often T-cells, in response to the one or more peptides administered to the subject. In some cases, the activity of immune cells may be detected using methods to determine antibody production, often auto-reactive antibodies (e.g., IgG), methods to detect immune cells, often T-cells (e.g., autoreactive regulatory T-cells) in the subject following administration of the one or more peptides or nucleic acids encoding the one or more peptides. Exemplary methods to determine antibody production or detect immune cells may include, but are not limited to, standard in vitro or in vivo immunological assays, such as enzyme linked immunosorbant assay (ELISA) or enzyme linked immunosorbant spot (ELIPOT) assays, delayed type hypersensitivity responses (DTH) and lymphocyte proliferation or cytoxicity assays may also be used.

The methods, systems, and processes may further comprise identifying putative epitopes from the one or more putative peptides, often a set of peptides (e.g., self-peptides). (FIG. 1, 140). In some cases, the one or more putative epitopes may be ranked, often the epitopes are ranked according to one or more parameters. In some cases, the parameters may include an affinity (e.g., high) of the putative epitopes for binding to MHCII molecules. For example, binding to MHCII molecules may include binding with high affinity across multiple HLA-DR alleles. In some cases, identifying putative epitopes may include performing binding assays, often standard competitive inhibition binding assays and/or by performing epitope mapping, often in silico. For example, each parameter and assay performed using each putative epitope may render a value, often the values are considered and a ranking applied to each putative epitope based on the values. In some cases, the putative epitopes may be ranked into quartiles. In some cases, epitopes selected for further analysis using the methods described herein may rank in the highest quartile.

The methods, systems, and processes may further comprise determining the potential of each ranked epitope to elicit an immune response in a subject. (FIG. 1, 160). In some cases, the immune response may include identifying immune cells, often T-cells, which secrete cytokines, often interferon-gamma (IFNγ) and/or interleukin-10 (IL-10). For example, immune cells may be identified using samples of cells isolated from a subject after administration of the ranked epitopes to the subject. In some cases, the subject has a disease. In other cases, the subject is a control (e.g., does not have a disease). In some cases, immune cells may be identified using standard immunological assays, for example, but not limited to, an ELISPOT assay, an ELISA, rtPCR analysis of mRNA expression, immunohistochemistry, or fluorescence in situ hybridization analysis (FISH). In some cases, secretion of cytokines (e.g., IFNγ and/or IL-10) may be quantified. In some cases, a ratio of the amount of IFNγ and IL-10 secreted by immune cells may be determined. The ratio may indicate ranked epitopes that induce a sub-type of an immune response. For example, the ratio may indicate that a ranked epitope induces Type 1 (Th1) responses (e.g., antigen specific responses). For another example, the ratio may indicate that a ranked epitope induces Type 2 (Th2) responses (e.g., antigen specific responses).

The methods, systems, and processes may further comprise identifying ranked epitopes that may induce a specific type of immune response in a subject, often the specific immune response is a Th1 response. In some cases, the ranked epitopes may be presented to immune cells of the subject on endogenous antigen presenting cells (APC). Identifying may further comprise creating T-cell lines, often epitope specific T-cells line, using the identified ranked epitopes according to standard immunological methods that may be, for example, but are not limited to, ranked epitopes may be identified such that the identified ranked epitope elicits an epitope and/or a peptide specific immune response in a subject. In some cases, the identified ranked epitopes may be further identified by a class of binding epitopes. For example, binding classes may be class I or class II. The binding class of an epitope may be identified using methods, for example but are not limited to the binding class of an epitope (e.g., Class I MHC or Class II MHC) may be identified by conducting blocking assays, often using the generated T-cell lines. In some cases, the T-cell lines may be an exogenous T-cell engineered to express a Chimeric Antigen Receptor construct that binds the epitope with high selectivity and avidity.

The disclosure describes methods, systems, and processes for the identification of putative peptides that may elicit an immune response in a subject. The methods, systems, and processes further include screening putative peptides to determine portions of the peptides containing peptide epitopes which may be the antigens eliciting the immune response in the subject. In some cases, the peptides may be human peptides. In some cases, the peptides may be associated with a disease, for example, the peptides may be differentially expressed in a subject with a disease. For example, peptides associated with a disease may be peptides which are upregulated (e.g., expression is increased relative to a control) in a subject with a disease. In some cases, the control may be a subject without the disease. For example, peptides associated with a disease may be peptides which are downregulated (e.g., expression is decreased relative to a control) in a subject with a disease. In some cases, the control may be a subject without the disease. In other cases, the peptides may be associated with a disease, for example, the peptides may be differentially expressed in a subject prior to the subject having the disease. For example, peptides associated with a disease may be peptides which are upregulated (e.g., expression is increased relative to a control) in a subject prior to the subject having the disease. In some cases, the control may be a subject without the disease. For example, peptides associated with a disease may be peptides which are downregulated (e.g., expression is decreased relative to a control) in a subject prior to the subject having the disease.

The methods, systems, and processes described herein further include determining an amino acid sequence of the peptide epitopes which may be the antigens eliciting the immune response in the subject. In some cases, the amino acids of the peptide epitopes may be isolated from a subject, often the isolated amino acids are purified from the subject. Techniques known to one of ordinary skill in the art may be used to isolate and/or purify amino acids from a subject or amino acid sequences may be synthesized. Methods known to those of ordinary skill in the art to obtain isolated and/or purified amino acid sequences derived from ex vivo translation of nucleic acid sequences may be used herein. In some cases, amino acid sequences of the peptides may be incorporated into compositions, often pharmaceutical compositions, for example vaccines, for administration to a subject in need thereof.

The methods, systems, and processes may also include determining the nucleic acid sequence which encodes the amino acids of the peptide epitopes. In some cases, the nucleic acid sequences encoding the amino acids of the peptide epitopes may be isolated from a subject, often the isolated nucleic acids are purified from the subject. Techniques known to one of ordinary skill in the art may be used to isolate and/or purify nucleic acids from a subject, for example, nucleic acid purification, polymerase chain reaction, nucleic acid synthesis and the like. Nucleic acids may also be synthesized. In some cases, the isolated and/or purified nucleic acids may be incorporated into a nucleic acid plasmid for expression in a subject. Plasmids containing nucleic acid sequences of at least one antigenic peptide epitope may be incorporated into compositions, often pharmaceutical compositions, for example vaccines, for administration to a subject in need thereof.

The peptide epitopes identified and selected for design into compositions, often vaccines, using the methods described herein may regulate the immune response of a subject to at least one peptide encoded by a nucleic acid or to a peptide delivered to the subject (FIG. 1, 180). In some cases, the immune response of a subject may be elicited in response to more than one peptide, often a set of peptides. For example, the peptide epitopes identified and selected may be designed to induce, entrain, and/or amplify or attenuate, suppress, or eliminate the immune response of a subject to one or more peptides. Often the peptides are human peptides. In some cases, the peptides are a specific set of peptides (e.g., human self-peptides).

Using the methods, systems, and processes described herein, the identified peptide epitopes may be incorporated into a composition, often a vaccine. For example, nucleic acids encoding the amino acid sequences of at least one peptide epitope contained within at least one plasmid may be incorporated into a vaccine. For another example, amino acids encoding at least one peptide epitope may be compounded into a vaccine. In some cases, the vaccine compositions may be optimized using the methods described herein such that upon administration to a subject, the vaccine composition may induce, amplify or entrain a protective immune response in a subject in need. For example, in some cases, the methods may be used to identify peptide epitopes for vaccine compositions that may be designed to induce, amplify or entrain immune responses against tumors. In other cases, the vaccine compositions may be optimized using the methods described herein such that upon administration to a subject, the vaccine composition may suppress, attenuate or eliminate a pathological one, in a subject in need thereof. For example, vaccine compositions may be designed to suppress, attenuate or eliminate immune responses contributing to the onset and/or progression of autoimmune diseases or any other disease state that is associated with the aberrant immune response to self-antigens.

The immune response elicited by epitopes, antigens, peptides and/or peptides described herein may be a sub-type of an immune response, often a Type I (Th1) and/or a Type 2 (Th2) response. In some cases, a Th1 response may be desired. In other cases, a Th2 response may be desired. In some cases, both a Th1 response and a Th2 response may be desired. The methods described herein further comprise determining one or more identified ranked epitopes which elicit a desired response in a subject after administration of the ranked epitopes.

In some cases, the immune response may be a response to an immunization administered to a subject using the composition comprising ranked epitopes. For example, the ranked epitopes may be epitopes from peptides of self-antigens (e.g., self-tumor antigens). In some cases, the immunization may induce activation of immune cells, often T-cells. Activation of a plurality of sub-types of T-cells may be induced. For example, T-regulatory cells are a sub-type of immune cells and may inhibit proliferation of other sub-types of immune cells, often Type I CD4+T-helper (Th1) and CD8+ cytotoxic T-cells.

The methods, systems, and processes described herein may include selection of ranked epitopes for compositions to prevent or treat disease in a subject. For example, disease may include cancer, autoimmune disease, antigen-induced inflammatory condition and the like. In some instances, the cancer may be a solid tumor or a hematologic malignancy. In some instances, the solid tumor may include sarcoma or carcinoma. Carcinoma may include, for example, breast cancer, colon cancer, gastroenterological cancer, kidney cancer, lung cancer, ovarian cancer, pancreatic cancer, or prostate cancer. In some cases, the epitopes make be ranked according to the sub-type of immune response elicited in a subject. For example, epitopes may be ranked as Th1 or Th2 as described herein. In some cases, ranked epitopes may be selected for compositions to prevent or treat disease in a subject, the selection of Th1 and/or Th2 ranked epitopes in accordance with achieving a desired immune response of a subject to the composition. In some cases, ranked epitopes may be added to or deleted from a composition targeting a disease to enhance efficacy of the composition. For example, a cancer vaccine comprising ranked epitopes targeting a peptide (e.g., IGFBP-2) may be modified such that specific ranked epitopes eliciting an undesirable response in a subject are removed from the composition and/or specific ranked epitopes eliciting a desirable response in a subject are added to the composition which may enhance the anti-cancer efficacy of the vaccine.

The methods, systems, or processes described herein may further include tuning a length of at least one putative epitope such that the putative epitope may comprise a nucleic acid sequence encoding an amino acid sequence which is longer than the minimum amino a nucleic acid sequence encoding an amino acid sequence of the putative epitope necessary to elicit a desired immune response. In some cases, the minimum nucleic acid sequence encoding an amino acid sequence of the putative epitope may be less than three, less than five, less than seven, less than 10, less than 12, less than 15, less than 17, less than 20, less than 22, less than 25, less than 27, less than 30, less than 32, less than 35, less than 37 or less than 40 amino acids in length. In some cases, a longer putative epitope may enhance the desired immune response following administration of the longer putative epitope to a subject. In some cases, the longer putative epitope may contain nucleic acids encoding at least one amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, 40 amino acids, 41 amino acids, 42 amino acids, 43 amino acids, 44 amino acids, 45 amino acids, 46 amino acids, 47 amino acids, 48 amino acids, 49 or at least 50 amino acids which exceed the minimum number of nucleic acids encoding the amino acids in the putative epitope.

The methods, systems, or processes described herein may further include tuning a length of at least one putative epitope such that the putative epitope may comprise an amino acid sequence which is longer than the minimum amino acid sequence of the putative epitope necessary to elicit a desired immune response. In some cases, the minimum amino acid sequence of the putative epitope may be less than three, less than five, less than seven, less than 10, less than 12, less than 15, less than 17, less than 20, less than 22, less than 25, less than 27, less than 30, less than 32, less than 35, less than 37 or less than 40 amino acids in length. In some cases, a longer putative epitope may enhance the desired immune response following administration of the longer putative epitope to a subject. In some cases, the longer putative epitope may contain at least one amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, 40 amino acids, 41 amino acids, 42 amino acids, 43 amino acids, 44 amino acids, 45 amino acids, 46 amino acids, 47 amino acids, 48 amino acids, 49 or at least 50 amino acids which exceed the minimum number of amino acids in the putative epitope.

The methods described herein include a method for designing a plasmid vaccine, the method comprising: determining a potential of a set of putative epitopes to induce a sub-type of an immune response; ranking a plurality of putative epitopes from the set of putative epitopes by the sub-type of the immune response; from the plurality of ranked putative epitopes, identifying a set of desired epitopes such that the set of desired epitopes induces a desired sub-type of an immune response in a subject; and arranging the desired epitopes to provide a plasmid vaccine design. In some cases, the set of putative epitopes comprise a set of epitopes of self-proteins of the subject. In some cases, the set of putative epitopes contains epitopes from between about 2 and about 50 unique peptides.

The methods described herein further include a method for designing a peptide vaccine, in which the method comprises determining a potential of a set of putative epitopes to induce a sub-type of an immune response; ranking a plurality of putative epitopes from the set of putative epitopes by the sub-type of the immune response; from the plurality of ranked putative epitopes, identifying a set of desired epitopes such that the set of desired epitopes induces a desired sub-type of an immune response in a subject; and arranging the desired epitopes to provide a peptide vaccine design.

The methods described herein, in some cases, include a subject. In some cases, the subject is a human. In some cases, the human has a disease. In some cases, the human is a healthy individual. In some cases, the set of putative epitopes is overexpressed in a subject with a disease compared to a subject without a disease.

The systems described herein include a system for designing a plasmid vaccine, which comprises a digital processing device comprising an operating system configured to perform executable instructions, and an electronic memory; a set of putative epitopes stored in the electronic memory; a computer program including instructions executable by the computer to create an application comprising: (i) a first software module configured to determine the potential of each putative epitope within the set of putative epitopes to induce a sub-type of an immune response; (ii) a second software module configured to rank a plurality of putative epitopes from the set of putative epitopes by the sub-type of the immune response, and identify a set of desired epitopes from the ranking, wherein the set of desired epitopes is capable of inducing a desired sub-type of an immune response in a subject; and (iii) a third software module configured to design a plasmid vaccine from the set of desired epitope identified in step (ii).

The systems describes herein further include a system for designing a peptide vaccine, which comprises a digital processing device comprising an operating system configured to perform executable instructions, and an electronic memory; a set of putative epitopes stored in the electronic memory; a computer program including instructions executable by the computer to create an application comprising: (i) a first software module configured to determine the potential of each putative epitope within the set of putative epitopes to induce a sub-type of an immune response; (ii) a second software module configured to rank a plurality of putative epitopes from the set of putative epitopes by the sub-type of the immune response, and identify a set of desired epitopes from the ranking such that the set of desired epitopes is capable of inducing a desired sub-type of an immune response in a subject; and (iii) a third software module configured to design a peptide vaccine from the set of desired epitope identified in step (ii).

The processes described herein include a plasmid vaccine designed by the process of determining the potential of each putative epitope within the set of putative epitopes to induce a sub-type of an immune response; ranking a plurality of putative epitopes from the set of putative epitopes by the sub-type of the immune response; identifying a set of desired epitopes from the ranking such that the set of desired epitopes is capable of inducing a desired sub-type of an immune response in a subject; and designing a plasmid vaccine from the set of desired epitope.

In some cases, the set of putative epitopes comprise a set of epitopes of self-proteins of the subject. In some cases, the set of putative epitopes contains epitopes from between about 2 and about 50 unique peptides. In some cases, the set of putative epitopes is overexpressed in a subject with a disease compared to a subject without a disease. In some cases, the process further comprises identifying the set of putative epitopes by a process selected from: a literature search, a database search, a search of bioinformatics mediums, an analysis of a fluid sample from a subject, an analysis of a cellular sample from a subject, an analysis of a tissue sample from a subject, or a combination thereof. In some cases, the process further comprises identifying the set of putative epitopes using a computer equipped with executable instructions. In some cases, the process further comprises ranking the plurality of putative epitopes from the set of putative epitopes by identifying an adaptive immune response to the set of putative peptides in a subject. In some cases, the process further comprises ranking the plurality of putative epitopes from the set of putative epitopes by using a computer equipped with executable instructions. In some cases, the sub-type of the immune response is selected from: production of IgG antibodies, production of specific Th cells in response to at least the first set of putative peptides, or a combination thereof. In some cases, the sub-type of the immune response is identified by an assay selected from: an enzyme linked immunosorbant assay (ELISA), an enzyme linked immunosorbant spot (ELISPOT) assay, a delayed type hypersensitivity responses (DTH), a lymphocyte proliferation or a cytoxicity assay, or a combination thereof. In some cases, the ranking includes ranking each epitope in the set of putative epitopes according to a parameter selected from: binding of each epitope to major histocompatibility complex (MHC) alleles, affinity of each epitope for major histocompatibility complex (MHC) alleles, or a combination thereof. In some cases, each epitope ranked in the top two quartiles of the set of putative epitopes is identified in the set of desired epitopes. In some cases, the sub-type of the immune response is a Type I immune response. In some cases, the Type I response is determined by measuring production of interferon gamma (IFNγ), interleukin-12 (IL-12), TNFα, or GM-CSF in the subject. In some cases, the sub-type of the immune response is a Type II immune response. In some cases, the Type II response is determined by measuring production of interleukin-10 (IL-10), interleukin-4 (IL-4), interleukin-5 (IL-5), or interleukin-6 (IL-6) in the subject. In some cases, each epitope within the set of putative epitopes may be differentiated by induction of a Type I immune response. In some cases, each epitope within the set of putative epitopes is differentiated by suppression of a Type I immune response. In some cases, each epitope within the set of putative epitopes is differentiated by induction of a Type II immune response. In some cases, the set of desired epitopes are presented on antigen presenting cells (APC)s in the subject. In some cases, the arranging of the desired epitopes comprises separating two or more epitopes with a sequence of linker nucleic acids. In some cases, the arranging of the desired epitopes comprises arranging two or more adjacent epitopes.

The processes described herein further include a peptide vaccine designed by the process of determining the potential of each putative epitope within the set of putative epitopes to induce a sub-type of an immune response; ranking a plurality of putative epitopes from the set of putative epitopes by the sub-type of the immune response; identifying a set of desired epitopes from the ranking such that the set of desired epitopes is capable of inducing a desired sub-type of an immune response in a subject; and designing a peptide vaccine from the set of desired epitope.

In some instances, the set of putative epitopes comprise a set of epitopes of self-proteins of the subject. In some instances, the set of putative epitopes contains epitopes from between about 2 and about 50 unique peptides. In some instances, the set of putative epitopes is overexpressed in a subject with a disease compared to a subject without a disease. In some instances, the process further comprises identifying the set of putative epitopes by a process selected from: a literature search, a database search, a search of bioinformatics mediums, an analysis of a fluid sample from a subject, an analysis of a cellular sample from a subject, an analysis of a tissue sample from a subject, or a combination thereof. In some instances, the process further comprises identifying the set of putative epitopes using a computer equipped with executable instructions. In some instances, the process further comprises ranking the plurality of putative epitopes from the set of putative epitopes by identifying an adaptive immune response to the set of putative peptides in a subject. In some instances, the process further comprises ranking the plurality of putative epitopes from the set of putative epitopes by using a computer equipped with executable instructions. In some instances, the sub-type of the immune response is selected from: production of IgG antibodies, production of specific Th cells in response to at least the first set of putative peptides, or a combination thereof. In some instances, the sub-type of the immune response is identified by an assay selected from: an enzyme linked immunosorbant assay (ELISA), an enzyme linked immunosorbant spot (ELISPOT) assay, a delayed type hypersensitivity responses (DTH), a lymphocyte proliferation or a cytoxicity assay, or a combination thereof. In some instances, the ranking includes ranking each epitope in the set of putative epitopes according to a parameter selected from: binding of each epitope to major histocompatibility complex (MHC) alleles, affinity of each epitope for major histocompatibility complex (MHC) alleles, or a combination thereof. In some instances, each epitope ranked in the top two quartiles of the set of putative epitopes is identified in the set of desired epitopes. In some instances, the sub-type of the immune response is a Type I immune response. In some instances, the Type I response is determined by measuring production of interferon gamma (IFNγ), interleukin-12 (IL-12), TNFα, or GM-CSF in the subject. In some instances, the sub-type of the immune response is a Type II immune response. In some instances, the Type II response is determined by measuring production of interleukin-10 (IL-10), interleukin-4 (IL-4), interleukin-5 (IL-5), or interleukin-6 (IL-6) in the subject. In some instances, each epitope within the set of putative epitopes may be differentiated by induction of a Type I immune response. In some instances, each epitope within the set of putative epitopes is differentiated by suppression of a Type I immune response. In some instances, each epitope within the set of putative epitopes is differentiated by induction of a Type II immune response. In some instances, the set of desired epitopes are presented on antigen presenting cells (APC)s in the subject. In some instances, the arranging of the desired epitopes comprises separating two or more epitopes with a sequence of linker nucleic acids. In some instances, the arranging of the desired epitopes comprises arranging two or more adjacent epitopes.

### Identification of Putative Peptides

The methods or processes may comprise identifying one or more putative peptides, often a set of peptides, for example self-peptides (e.g., about 2 to about 50 different peptides) such that expression of the one or more putative peptides may be associated with a disease. For example, associated with a disease may include increased expression in a subject with a disease compared to a subject without a disease, decreased expression in a subject with a disease compared to a subject without a disease, stable expression in a subject with a disease compared to a subject without a disease, increased expression in a subject that may develop a disease compared to a subject without a disease, decreased expression in a subject that may develop a disease compared to a subject without a disease or stable expression in a subject that may develop a disease compared to a subject without a disease. In some cases, the disease may be a prognosis, a pathophysiological condition or homeostatic state. For example, the disease may include, but is not limited to cancer, autoimmune disease and metabolic disease.

The putative peptides within the set of putative peptides identified using the methods or processes described herein may be members of the same peptide family. A peptide family may be a group of peptides categorized by any feature such that the feature categorized is a similar feature. For example, a peptide family may be a cancer, tumor, autoimmune, cytoskeletal, metabolic, glycolytic, stem cell, epithelial to mesenchymal transition, embryogenesis peptide family, invasion, migration, inhibition of apoptosis, cell survival, angiogenesis, proliferation, drug resistance, cancer stem cell maintenance, and evasion of immunologic defense mechanisms or the like.

In some cases, the method or process further comprises identifying the set of putative epitopes by a method selected from: a literature search, a database search, a search of bioinformatics mediums, analysis of a fluid sample from a subject, analysis of a cellular sample from a subject, analysis of a tissue sample from a subject, or a combination thereof. In some cases, the method or process further comprises identifying the set of putative epitopes using a digital processing device (e.g. a computer) comprising an operating system and equipped with executable instructions. In some cases, the method or process further comprises ranking the plurality of putative epitopes from the set of putative epitopes by a method selected from: a literature search, a database search, a search of bioinformatics mediums, analysis of a fluid sample from a subject, analysis of a cellular sample from a subject, analysis of a tissue sample from a subject, or a combination thereof. In some cases, the method or process further comprises ranking the plurality of putative epitopes from the set of putative epitopes by identifying an adaptive immune response to the set of putative peptides in a subject. In some cases, the method or process further comprises ranking the plurality of putative epitopes from the set of putative epitopes by using a computer equipped with executable instructions. In some cases, ranking includes ranking each epitope in the set of putative epitopes according to a parameter selected from: binding of each epitope to major histocompatibility complex (MHC) alleles, affinity of each epitope for major histocompatibility complex (MHC) alleles, or a combination thereof.

In some cases, the subject is a human. In some cases, the human has a disease. In some cases, the human is a healthy individual.

In some cases, the method or process further comprises identifying the set of putative epitopes by a method selected from: a literature search, a database search, a search of bioinformatics mediums, an analysis of a fluid sample from a subject, an analysis of a cellular sample from a subject, an analysis of a tissue sample from a subject, or a combination thereof. In some cases, the method further comprises identifying the set of putative epitopes using a computer equipped with executable instructions.

The methods or processes may further comprise identifying one or more putative peptides, often a set of peptides (e.g., self-peptides). Often, the one or more putative peptides may be identified with a digital processing device (e.g. computer) equipped with computer-readable medium and executable instructions. In some cases, the computer-readable medium may be a processor, memory and/or a hard drive. The amino acid sequences of one or more putative peptides may be entered into the digital processing device (e.g. computer) equipped with computer-readable medium and executable instructions. In some cases, the amino acid sequence of the putative peptide may be analyzed to identify one or more putative peptides which elicits an immune response in a subject. The digital processing device (e.g. computer) equipped with computer-readable medium and executable instructions may be connected to an internet, an intranet and/or and extranet. In some cases, the connection may be wireless, hard-wired, ethernet, bluetooth or the like. At least one database may be interrogated by the computer equipped with computer-readable medium and executable instructions such that the one or more putative peptides identified above may be analyzed for eliciting an immune response in a subject.

In some cases, the one or more putative peptides may be identified, and for example, identification methods may include, performing at least one of the following; a literature search, a database search, a search of bioinformatics mediums, analysis of a fluid sample from a subject, analysis of a cellular sample from a subject or analysis of a tissue sample from a subject. In some cases, the sample from a subject may be blood, other body fluids, tissue, cells or the like. In some cases, the sample may be isolated from a subject with a disease (e.g., a patient) and/or control subject (e.g., a subject without a disease).

In some cases, at least one putative peptide may be identified by performing a key word search in literature databases. For example, literature databases may include PubMed and the like. In some cases, a key word search may include one key word. In other cases, may include more than one key word, for example, two, three, four, five, six, seven, eight, nine, ten, 11, 12, 13, 14, 15, 16, 17, 18, 19, 20, 21, 22, 23, 24, 25, 26, 27, 28, 29, 30 or more than 30 key words. The key word search in the literature database may result in a first set of results, such that the first set includes at least one item of literature. Often, the key word search results in a first set of results comprising more than one, for example, more than 10, more than 50, more than 100, more than 150, more than 200, more than 250, more than 300, more than 350, more than 400, more than 450, more than 500, more than 550, more than 600, more than 650, more than 700, more than 750, more than 800, more than 850, more than 900, more than 1000, more than 1200, more than 1400, more than 1600, more than 1800, more than 2000, more than 22000, more than 2400, more than 2600, more than 2800, more than 3000, more than 3500, more than 4000, more than 4500, more than 5000, more than 5500, more than 6000, more than 6500, more than 7000, more than 7500, more than 8000, more than 8500, more than 9000, more than 9500 or more than 10,000 items of literature. In some cases, the literature results may be reviewed and an additional literature search may be performed using the first set of results to narrow the first set of results into a second set of results.

The literature results in the second set of results may be evaluated to identify at least one putative peptide. In some cases, the at least one putative peptide identified, as described above, may be ranked based on factors. For example, the factors may include upregulated, downregulated, overexpressed or underexpressed in a subject with a disease or in a subject that may develop, association with transformation from one cell type to a different cell type, for example, epithelial to mesenchymal transformation, association with stem cells, often cancer stem cells, and/or expression indicates a poor prognosis of a subject with a disease or that may develop a disease by univariate and/or multivariate analysis.

The methods or processes described herein may further determine a sub-type of an immune response elicited by putative epitopes. One or more than one peptide of a putative epitope, often a set of peptides of putative epitopes, may be generated for administration to a subject. In some cases, a subject can be a human, mouse, rat, dog, pig, guinea pig, cow, horse, chicken, rabbit, monkey, baboon, orangutan or a gorilla. In some cases, the subject may be a subject in need.

The one or more than one peptide of a putative epitope may comprise a human amino acid sequence. In some cases, the peptide of a putative epitope may comprise a non-human amino acid sequence from a mouse amino acid sequence, rat amino acid sequence, dog amino acid sequence, pig amino acid sequence, guinea pig amino acid sequence, cow amino acid sequence, horse amino acid sequence, chicken amino acid sequence, rabbit amino acid sequence, monkey amino acid sequence, baboon amino acid sequence, orangutan amino acid sequence or a gorilla amino acid sequence. The non-human amino acid sequence of a putative epitope may be homologous to the human amino acid sequence of a putative epitope. In some cases, the non-human amino acid sequence may be 100% homologous to the human amino acid sequence. In other cases, the non-human amino acid sequence may be more than 50%, more than 55%, more than 60%, more than 65%, more than 70%, more than 75%, more than 80%, more than 85%, more than 86%, more than 87%, more than 88%, more than 89%, more than 90%, more than 91%, more than 92%, more than 93%, more than 94%, more than 95%, more than 96%, more than 97%, more than 98 or more than 99% homologous to the human amino acid sequence. In some cases, the non-human amino acid sequences may be 100% homologous to the human amino acid sequence. In other cases, the non-human amino acid sequence may be more than 50%, more than 55%, more than 60%, more than 65%, more than 70%, more than 75%, more than 80%, more than 85%, more than 86%, more than 87%, more than 88%, more than 89%, more than 90%, more than 91%, more than 92%, more than 93%, more than 94%, more than 95%, more than 96%, more than 97%, more than 98 or more than 99% homologous to the other non-human amino acid sequences.

The one or more than one peptide of a putative epitope may comprise a nucleic acid sequence encoding a human amino acid sequence. In some cases, the peptide of a putative epitope may comprise a nucleic acid sequence encoding a non-human amino acid sequence from a nucleic acid sequence encoding a mouse amino acid sequence, a nucleic acid sequence encoding a rat amino acid sequence, a nucleic acid sequence encoding a dog amino acid sequence, a nucleic acid sequence encoding a pig amino acid sequence, a nucleic acid sequence encoding a guinea pig amino acid sequence, a nucleic acid sequence encoding a cow amino acid sequence, a nucleic acid sequence encoding a horse amino acid sequence, a nucleic acid sequence encoding a chicken amino acid sequence, a nucleic acid sequence encoding a rabbit amino acid sequence, a nucleic acid sequence encoding a monkey amino acid sequence, a nucleic acid sequence encoding a baboon amino acid sequence, a nucleic acid sequence encoding an orangutan amino acid sequence or a nucleic acid sequence encoding a gorilla amino acid sequence. The non-human nucleic acid sequence encoding an amino acid sequence of a putative epitope may be homologous to the human amino acid sequence of a putative epitope. In some cases, the non-human nucleic acid sequence encoding an amino acid sequence may be 100% homologous to the human amino acid sequence. In other cases, the non-human nucleic acid sequence encoding an amino acid sequence may be more than 50%, more than 55%, more than 60%, more than 65%, more than 70%, more than 75%, more than 80%, more than 85%, more than 86%, more than 87%, more than 88%, more than 89%, more than 90%, more than 91%, more than 92%, more than 93%, more than 94%, more than 95%, more than 96%, more than 97%, more than 98 or more than 99% homologous to the nucleic acid sequence encoding a human amino acid sequence. In some cases, the nucleic acid sequence encoding non-human amino acid sequence may be 100% homologous to the nucleic acid sequence encoding the human amino acid sequence. In other cases, the non-human nucleic acid sequence encoding an amino acid sequence may be more than 50%, more than 55%, more than 60%, more than 65%, more than 70%, more than 75%, more than 80%, more than 85%, more than 86%, more than 87%, more than 88%, more than 89%, more than 90%, more than 91%, more than 92%, more than 93%, more than 94%, more than 95%, more than 96%, more than 97%, more than 98 or more than 99% homologous to the other non-human nucleic acid sequence encoding the amino acid sequences.

In some cases, the method or process further comprises ranking the plurality of putative epitopes from the set of putative epitopes by a method selected from: a literature search, a database search, a search of bioinformatics mediums, analysis of a fluid sample from a subject, analysis of a cellular sample from a subject, analysis of a tissue sample from a subject, or a combination thereof. In some cases, the method or process further comprises ranking the plurality of putative epitopes from the set of putative epitopes by identifying an adaptive immune response to the set of putative peptides in a subject. In some cases, the method or process further comprises ranking the plurality of putative epitopes from the set of putative epitopes by using a computer equipped with executable instructions. In some cases, ranking includes ranking each epitope in the set of putative epitopes according to a parameter selected from: binding of each epitope to major histocompatibility complex (MHC) alleles, affinity of each epitope for major histocompatibility complex (MHC) alleles, or a combination thereof.

### Determining Antigenicity of Putative Peptides

The methods or processes may further comprise determining the antigenicity of one or more identified putative peptides in a subject, often a human subject. In some cases, the antigenicity may be determined by the detection of an immune response elicited by a subject following administration of nucleic acids encoding the one or more putative identified peptides or the one or more putative identified peptides to the subject. For example, the immune response may be detected by determining the activity of immune cells, often T-cells, in response to the one or more peptides administered to the subject. The immune response that may be elicited by peptides described herein may be a sub-type of an immune response, often a Type I (Th1) and/or a Type 2 (Th2) response. In some cases, a Th1 response may be desired. In other cases, a Th2 response may be desired. In some cases, both a Th1 response and a Th2 response may be desired.

In some cases, the immune response may be a response following administration of a putative peptide or of nucleic acids encoding the one or more putative identified peptides identified using the methods described herein. In some cases, the immunization may induce activation of immune cells, often T-cells. Activation of a plurality of sub-types of T-cells may be induced in response to administration of the putative peptide. For example, T-regulatory cells are a sub-type of immune cells and may inhibit proliferation of other sub-types of immune cells, often Type I CD4+T-helper (Th1) and CD8+ cytotoxic T-cells. For example, peptides may be ranked as Th1 or Th2 as described herein. In some cases, peptides may be selected for compositions to prevent or treat disease in a subject, the selection of Th1 and/or Th2 peptides in accordance with achieving a desired immune response of a subject to the composition. In some cases, peptides may be added to or deleted from a composition targeting a disease to enhance efficacy of the composition. In some cases, the peptides included in a composition administered to a subject may be selected based upon the sub-type of the immune response elicited by the subject. For example, if a Th1 immune response is desired, peptides that elicit a Th1 response may be included in the composition and peptides that elicit a Th2 response may be omitted from the composition. For another example, if a Th2 immune response is desired, peptides that elicit a Th2 response may be included in the composition and peptides that elicit a Th1 response may be omitted from the composition.

In some cases, each epitope within the set of putative epitopes may be differentiated by induction of a Type I immune response. In some cases, each epitope within the set of putative epitopes may be differentiated by suppression of a Type I immune response. In some cases, each epitope within the set of putative epitopes may be differentiated by induction of a Type II immune response. In some cases, the desired sub-type of immune response is characterized by a ratio of Type I cytokine production to Type II cytokine production that is greater than 1. In some cases, the desired sub-type of immune response is characterized by a ratio of Type I cytokine production to Type II cytokine production that is less than 1.

The methods or processes described herein further include, in some cases, the sub-type of the immune response is selected from: production of IgG antibodies, production of specific Th cells in response to at least the first set of putative peptides, or a combination thereof. In some cases, the sub-type of the immune response is identified by an assay selected from: an enzyme linked immunosorbant assay (ELISA), an enzyme linked immunosorbant spot (ELISPOT) assay, a delayed type hypersensitivity responses (DTH), a lymphocyte proliferation or a cytoxicity assay, or a combination thereof. In some cases, IFNγ is measured using an assay selected from: ELISPOT assay, ELISA, rtPCR analysis of mRNA expression, immunohistochemistry, fluorescence in situ hybridization analysis (FISH), or a combination thereof. In some cases, IL-10 is measured using an assay selected from: ELISPOT assay, ELISA, rtPCR analysis of mRNA expression, immunohistochemistry, and fluorescence in situ hybridization analysis (FISH), or a combination thereof.

In some cases, the activity of immune cells may be detected using methods to determine antibody production (e.g., IgG), often auto-reactive antibodies, methods to detect immune cells, often T-cells (e.g., autoreactive regulatory T-cells) in the subject following administration of the one or more putative peptides. Putative peptides may be commercially available, prepared by custom order or synthesized in a private lab. In some cases, antibodies may be specific for peptides expressed by a subject with a disease. In other cases, antibodies may be specific for peptides expressed by a subject that may develop a disease. Methods to determine antibody production or detect immune cells may include for example, standard in vitro or in vivo immunological assays, such as direct enzyme linked immunosorbant assay (ELISA), indirect ELISA, enzyme linked immunosorbant spot (ELISPOT) assays, Western blot assays, delayed type hypersensitivity responses (DTH) and lymphocyte proliferation or cytoxicity assays may also be used. In some cases, any of the above immunologic assays may be commercially available, prepared by custom order or synthesized in a private lab.

In some cases, the sub-type of the immune response is selected from: production of IgG antibodies, production of specific Th cells in response to at least the first set of putative peptides, or a combination thereof. In some cases, the sub-type of the immune response is identified by an assay selected from: an enzyme linked immunosorbant assay (ELISA), an enzyme linked immunosorbant spot (ELISPOT) assay, a delayed type hypersensitivity responses (DTH), a lymphocyte proliferation or a cytoxicity assay, or a combination thereof. In some cases, the sub-type of the immune response is a Type I immune response. In some cases, the sub-type of the immune response is a Type II immune response. In some cases, the Type I response is determined by measuring production of interferon gamma (IFNγ), interleukin-12 (IL-12), tumor necrosis factor alpha (TNFα), or GM-CSF in the subject. In some cases, the Type II response is determined by measuring production of interleukin-10 (IL-10), interleukin-4 (IL-4), interleukin-5 (IL-5), or interleukin-6 (IL-6) in the subject.

In some cases, IFNγ is measured using an assay selected from: ELISPOT assay, ELISA, rtPCR analysis of mRNA expression, immunohistochemistry, fluorescence in situ hybridization analysis (FISH), or a combination thereof. In some cases, IL-10 is measured using an assay selected from: ELISPOT assay, ELISA, rtPCR analysis of mRNA expression, immunohistochemistry, and fluorescence in situ hybridization analysis (FISH), or a combination thereof.

In some cases, the set of desired epitopes are presented on antigen presenting cells (APCs) in the subject. In some cases, the APCs in the subject are endogenous. Serum may be screened for reactivity to at least one putative peptide using the methods described herein, often serum may be isolated from at least one subject and immunologic assays performed on freshly isolated serum, purified serum, previously frozen isolated serum and/or previously frozen purified serum. In some cases, serum may be drawn at the time a subject is initially diagnosed with a disease, a time after the subject is diagnosed with a disease, prior to a subject receiving treatment for a disease, while a subject is receiving treatment for a disease, after a subject has received treatment for a disease, before a subject develops a disease, from a subject without a disease or from a control subject. In some cases, at least one putative peptide may be identified using the methods described herein. For example, one putative peptide may be identified, often more than one peptide, more than two peptides, more than three peptides, more than four peptides, more than five peptides, more than six peptides, more than seven peptides, more than eight peptides, more than nine peptides, more than ten peptides, more than 11 peptides, more than 12 peptides, more than 13 peptides, more than 14 peptides, more than 15 peptides, more than 16 peptides, more than 17 peptides, more than 18 peptides, more than 19 peptides, more than 20 peptides, more than 21 peptides, more than 22 peptides, more than 23 peptides, more than 24 peptides, more than 25 peptides, more than 26 peptides, more than 27 peptides, more than 28 peptides, more than 29 peptides, more than 30 peptides, more than 31 peptides, more than 32 peptides, more than 33 peptides, more than 34 peptides, more than 35 peptides, more than 36 peptides, more than 37 peptides, more than 38 peptides, more than 39 40 peptides, more than 41 peptides, more than 42 peptides, more than 43 peptides, more than 44 peptides, more than 45 peptides, more than 46 peptides, more than 47 peptides, more than 48 peptides, more than 49 or more than 50 peptides.

In some cases, identified peptides may be candidate peptides and further screened to identify putative epitopes which elicit a sub-type of an immune response in a subject. A candidate peptide may be an antigen eliciting an immune response in a subject if any of the serum samples screened as described above yield a positive result. The methods may further include a statistical analysis. For example, with 144 subjects, if the estimated proportion is 50%, a 95% confidence may be determined such that the estimate may be within 0.08 of the true proportion. If the estimated proportion is 90%, a 95% confidence may be determined such that the estimate may be within 0.05 of the true proportion.

In some cases, each epitope ranked in the top two quartiles of the set of putative epitopes is identified in the set of desired epitopes. In some cases, the affinity of each epitope for MHC alleles is high across a plurality of human leukocyte antigen (HLA) alleles.

### Identifying Putative Epitopes of Putative Peptides Eliciting an Immune Response

The methods or processes may further comprise identifying putative epitopes from the one or more putative peptides, often a set of peptides (e.g., self-peptides). Often, putative epitopes from one or more putative peptides may be identified with a digital processing device (e.g. computer) equipped with computer-readable medium and executable instructions. In some cases, the computer-readable medium may be a processor, memory and/or a hard drive. The amino acid sequences of one or more putative peptides may be entered into the computer equipped with computer-readable medium and executable instructions. In some cases, one or more portions of the amino acid sequence of the putative peptide may be analyzed to identify one or more portions of the putative peptide which elicits an immune response in a subject. The digital processing device (e.g. computer) equipped with computer-readable medium and executable instructions may be connected to an internet, an intranet and/or and extranet. In some cases, the connection may be wireless, hard-wired, ethernet, bluetooth or the like. At least one database may be interrogated by the computer equipped with computer-readable medium and executable instructions such that the one or more putative portions of the putative peptide identified above may be analyzed for eliciting an immune response in a subject.

In some cases, the one or more putative epitopes may be ranked, using the digital processing device (e.g. computer) equipped with computer-readable medium and executable instructions, often the epitopes are ranked according to one or more parameters. In some cases, the parameters may include an affinity (e.g., high) of the putative epitopes for binding to MHCII molecules. For example, binding to MHCII molecules may include binding with high affinity across multiple HLA-DR alleles. In some cases, identifying putative epitopes may include performing binding assays, often standard competitive inhibition binding assays and/or by performing epitope mapping, often in silico. For example, each parameter and assay performed using each putative epitope may render a value, often the values are considered and a ranking applied to each putative epitope based on the values. In some cases, the putative epitopes may be ranked into quartiles. In some cases, epitopes selected for further analysis using the methods described herein may rank in the highest quartile.

The methods or processes described herein include the identification of one or more putative epitopes from one or more putative peptides, often the putative peptides are a specific set of self-peptides of a subject. In some cases, identification may include determining a set of putative peptides which may contain putative epitopes predicted to bind to a receptor of interest, often the receptor is a major histocompatibility complex molecule of class II (MHCII). In some cases, the putative epitopes may be predicted to bind to at least one amino acid, at least two MHC allele, at least three MHC allele, at least four MHC allele, at least five MHC allele, at least six MHC allele, at least seven MHC allele, at least eight MHC allele, at least nine MHC allele, at least ten MHC allele, at least 11 MHC allele, at least 12 MHC allele, at least 13 MHC allele or at least 14 MHC allele. In some cases, MHC alleles may be human lymphocyte antigens (HLA) molecules. For example, the HLA alleles may be, but are not limited to, alleles of HLA-DR molecules, such as HLA-DRB1*0101, HLA-DRB1*0301, HLA-DRB1*0401, HLA-DRB1*0404, HLA-DRB1*0405, HLA-DRB1*0701, HLA-DRB1*0802, HLA-DRB1*0901, HLA-DRB1*1101, HLA-DRB1*1201, HLA-DRB1*1302, HLA-DRB1*1501, HLA-DRB4*0101 or HLA-DRB5*0101.

In some cases, nucleic acid sequences encoding amino acid sequences of putative epitopes may be the query sequences for input into databases, often in FASTA format, such that the formatting is compatible for use with the selected database. In other cases, amino acid sequences of putative epitopes may be the query sequences for input into databases, often in FASTA format. Any database containing nucleic acid and/or amino acid information known to one of ordinary skill in the art may be used, in an exemplary case, the National Center for BioInformatics database may be used.

In some cases, algorithms may be used to predict the MHC allele to which the putative epitope may bind. The algorithms may be publically, commercially or privately available. For example, the algorithms may be web-based, downloadable from the web or the like. Often, three algorithms may be used may be used to predict the MHC allele. For example, three algorithms may be SYFPEITHI, PROPRED and RANKPEP. In some cases, more than one, more than two, more than three, more than four, more than five, more than six, more than seven, more than eight, more than nine or more than ten algorithms may be used. In some cases, nucleic acid sequences encoding amino acid sequences of putative epitopes may be the query sequences for input into algorithms using any format known to one of ordinary skill in the art such that the input format is compatible with the algorithm, often FASTA format may be used. In other cases, amino acid sequences of putative epitopes may be the query sequences for input into algorithms using any input format is compatible with the algorithm, often FASTA format may be used.

At least one algorithm may be used to generate at least one score, often the score is assigned to a putative epitope. In some cases, the score may indicate binding of the putative epitope to at least one allele of an MHC molecule. For example, algorithm-generated epitope binding scores may be used to map putative epitopes within a larger peptide sequence. In some cases, the larger peptide sequence may be predicted to contain putative epitopes that may interact with at least one MHC allele. In some cases, the putative epitopes may be ranked based upon the score of each epitope, often the score for ranking each putative epitope may be categorized according to HLA alleles. In some cases, the top scoring epitopes for each HLA allele (e.g., HLA-DR) may be used to create a heat map. In some cases, about three, about four, about five, about six, about seven, about eight, about nine, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49 or about 50 top scoring epitopes may be used to create the heat map. Often, about twenty top scoring epitopes may be used to create a heat map. In some cases, the heat map may be a heat map of the query peptide.

Each algorithm may generate at least one score for each putative epitope, the at least one score subject to a scoring system. In some cases, each algorithm may have a unique scoring system. In other cases, each algorithm may have the same scoring system. In other cases, some algorithms may have a unique scoring system and other algorithms may have the same scoring system. At least one calculation may be applied to at least one score for each putative epitope. In some cases, the calculation may be a normalization. For example, at least one score for each putative epitope derived from at least one algorithm with a unique scoring system may be normalized such that more than one score where each score is the result of a different algorithm may be complied. In some cases, at least one score is normalized before compiling at least one score from at least one algorithm. In other cases, more than one, more than two, more than three, more than four, more than five, more than six, more than seven, more than eight, more than nine, more than ten, more than 11, more than 12, more than 13, more than 14, more than 15, more than 16, more than 17, more than 18, more than 19 or more than 20 scores may be normalized before compiling at least one score from at least one algorithm. A plurality of calculations may be applied to a numerical value (e.g., a score) such that the numerical value subject to the calculation is normalized may be used with the methods described herein. In some cases, at least one score may be normalized by dividing the top score obtained by at least one algorithm, often an epitope with the highest predicted affinity may have a normalized score of 1.0.

In some cases, each epitope ranked in the top two quartiles of the set of putative epitopes is identified in the set of desired epitopes. In some instances, the top two quartiles include the top 25% quartile or the 75%-100% percentile and the 50% to 75% quartile. In some cases, the affinity of each epitope for MHC alleles is high across a plurality of human leukocyte antigen (HLA) alleles. In some instances, the term “high” refers to a value, such as an IC₅₀ value. In some instances, the term “high” refers to an IC₅₀ value of from about 1 uM to about 1 pM, about 500 nM to about 50 pM, 50 nM to about 500 pM, or about 5 nM to about 1 nM. In some instances, the term “high” refers to a value, such as a value of greater than 1, greater than 1.5, greater than 2, greater than 2.5, greater than 3, or more.

Methods for compiling and analyzing data may be applied to a numerical value (e.g., a score) using the methods or processes described herein. In some cases, the data may be scores of epitopes, often the epitopes may be putative, ranked and/or desired. For example, the data may be epitope prediction data. In some cases, software may be used to compile and analyze data, for example, the software may be graphical, tabular and or text software. In some cases, the software may be Microsoft Excel, Microsoft Access, GraphPad Prizm and/or the like. The software may be programmed commercially, publically or privately, in some cases, the programming may occur prior to entering data into the software program. In other cases, the programming may occur as the data is entered into the software program. In some cases, programming may include the use of equations, functions and/or the like. For example, the software may be programmed such that (i) each amino acid of a putative epitope may be assigned a normalized score of the putative epitope, (ii) a number of different HLA alleles with epitopes at each amino acid position may be calculated and, in some cases, a graph may be generated, (iii) a sum of at least one normalized score from at least one putative epitope may be calculated and, often, may be graphed at least at one amino acid position, and (iv) a “Multiple Score” may be calculated and graphed. In some cases, the “multiple score” may be a product of a sum of the at least one normalized score and a number of HLA alleles.

The Multiple Score may be a score calculated using from measurements obtained using the methods described herein. In some cases, the Multiple Score may represent an epitope binding strength. In other cases, the Multiple Score may represent an epitope promiscuity. In yet other cases, the Multiple Score may represent both an epitope binding strength and an epitope promiscuity. The Multiple Score may be used to create a visual representation of an epitope binding strength and/or an epitope promiscuity. In some cases, the visual representation may be a heat map. For example, a heat map may depict at least one multiple score of at least one putative epitope from at least one putative peptide. Often, the heat map may be a graph of amino acid position versus Multiple Score. In some cases, the amino acid position may be plotted on the x-axis of the graph. In other cases, the amino acid position may be plotted on the y-axis of the graph. In some cases, the multiple score may be plotted on the x-axis of the graph. In other cases, the multiple score may be plotted on the y-axis of the graph. In an exemplary case, the amino acid position may be plotted on the x-axis of the graph and the multiple score may be plotted on the y-axis of the graph. In some cases, the heat map may be a MHC class II heat map.

Often amino acid sequences and/or nucleic acid sequences may be organized using at least one software application on a computer equipped with computer-readable medium and executable instructions, such that the amino acid sequences and/or nucleic acid sequences may be included in the visual representations, for example, a heat map. In some cases, the software applications may generate templates for heat maps. In some cases, the amino acid sequences and/or the nucleic acid sequences may be input into the software application in FASTA format. For example, the amino acid and/or nucleic acid sequences may be input in FASTA format into the vertical columns of the heat map. For example, the amino acid and/or nucleic acid sequences may be input in FASTA format into the horizontal columns of the heat map.

In some cases, the heat map may be generated using colors which indicate the Multiple Score value of an amino acid in the epitope. For example, the amino acids depicted in the heat map may be color-coded based on the Multiple Score values. In some cases, multiple score values may be color-coded by ranges, thresholds and the like. For example, a single color may be assigned to a range of Multiple Score values such that each single color may indicate Multiple Scores within the range of about 75-100%, about 50-75%, about 25-50% and about 10-25%. For another example, a single color may be assigned to a threshold of Multiple Score values such that each single color may indicate Multiple Scores of greater than 0% but less than 25%, about 25%-less than 50%, about 50%-less than 75%, about 75% to less than 100% or 100%. In some cases, the color-coded heat maps may aid in selection of the peptide sequences of the putative epitopes for further analysis, often with immunological assays.

Peptide sequences of the putative epitopes that may have been selected from heat maps may be constructed into peptides for further analysis. In some cases, the peptide sequences of the putative epitopes may be a percentage of the putative epitope. For example, the peptide sequences may be less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95% or less than 100% of the putative epitope.

### Determining a Sub-Type of an Immune Response Elicited by Putative Epitopes

Peptides of putative epitopes or nucleic acids encoding peptides of putative epitopes may be administered to a subject and the immune response elicited by peptides of putative epitopes or nucleic acids encoding peptides of putative epitopes may be determined. Often, the immune response may be determined using an immunological assay. In some cases, peptides of putative epitopes or nucleic acids encoding peptides of putative epitopes may be tested using one immunological assay. In other cases, peptides of putative epitopes or nucleic acids encoding peptides of putative epitopes may be tested using more than one immunological assay. For example, peptides of putative epitopes or nucleic acids encoding peptides of putative epitopes may be tested using one immunological assay, two immunological assays, three immunological assays, four immunological assays, five immunological assays, six immunological assays, seven immunological assays, eight immunological assays, nine immunological assays, ten immunological assays, 11 immunological assays, 12 immunological assays, 13 immunological assays, 14 immunological assays, 15 immunological assays, 16 immunological assays, 17 immunological assays, 18 immunological assays, 19 immunological assays, 20 or more than 20 immunological assays.

The methods or processes described herein may include generation of cell lines, often the cell lines are specific to a sub-type of an immune response, for example, a Th1 response and/or a Th2 response to one or more peptides of putative epitopes or nucleic acids encoding peptides of putative epitopes. In some cases, the peptide of interest may be the amino acid sequence of a non-human peptide. For example, cell lines from non-human subjects discussed above may be prepared from non-human subjects vaccinated with a peptide of interest. In some cases, the peptide of interest may be the putative peptide from which the putative epitopes were derived. In other cases, the peptide of interest may be related to the putative peptide from which the putative epitopes were derived. In other cases, the peptide of interest may be unrelated to the putative peptide from which the putative epitopes were derived. Often, the peptide of interest may be the amino acid sequence of a human peptide. For example, cell lines from human subjects discussed above may be prepared from human subjects vaccinated with a peptide of interest. In some cases, the peptide of interest may be the putative peptide from which the putative epitopes were derived. In other cases, the peptide of interest may be related to the putative peptide from which the putative epitopes were derived. In other cases, the peptide of interest may be unrelated to the putative peptide from which the putative epitopes were derived.

Any of the subjects described above may be stimulated with one or more peptides of putative epitopes or nucleic acids encoding peptides of putative epitopes prior to isolation of cells for generation of cell lines. In some cases, cells may be isolated from a subject following stimulation with a peptide of putative epitopes or nucleic acids encoding peptides of putative epitopes. Often, cells are isolated from a tissue of the subject, for example, the tissue may be the spleen, liver, lung, brain, bone marrow, skeletal muscle, blood, skin, lymph nodes or the like. For example, cells may be B cells, T-cells, macrophages, dendritic cells, monocytes, neutrophils, eosinophils, smooth muscle cells, stromal cells or any cell which is a precursor of the aforementioned cells. In an exemplary case, T-cell lines are prepared from the subject. Often, isolated cells are stimulated ex vivo with peptides of putative epitopes or nucleic acids encoding peptides of putative epitopes.

The methods or processes may further determine the phenotype of immune cells, often T-cells, isolated from a subject to which the peptides of the putative epitopes were administered. In some cases, expression of receptors (e.g., intracellular receptors, cell-surface receptors and/or internalized cell surface receptors) may be determined in the T-cells isolated from the subject. For example, immunoassays such as flow cytometry and/or Westernblotting may be performed using the methods described herein. Often, expression of immune cell markers (e.g., CD49b, CD4, CD3 and/or CD19) may be determined using antibodies detecting each of the aforementioned peptides. In some cases, antibodies may be directly labeled to a detection agent, often a fluorophore. For example, detection agents may include PE, APC, PerCP, FITC and PE-Cy7. Flow cytometry data may be analyzed using FlowJo software or the like.

In some cases, the methods or processes may further determine the activation of immune cells which elicit a sub-type of an immune response following administration of peptides of putative epitopes. For example, the activated immune cells may be disease-specific. In some cases, CD8+ T-cells, CD4+ T-cells, natural killer cells, macrophages, B cells, monocytes, dendritic cells or the like may be activated. The sub-type of the immune response may be determined by secretion of one or more cytokines by the activated immune cells, often IFNγ and/or IL-10.

A plurality of immunological assays may be used with the methods or processes described herein to test at least one peptides of putative epitopes or nucleic acids encoding peptides of putative epitopes to determine if the peptide of the putative epitope elicits an immune response, often the sub-type of the immune response may be determined, often the immunologic assay may be an ELISPOT assay, an ELISA assay, a multiplex assay or the like. For example, a suitable immunologic assay may include performing an ELISPOT assay using cells derived from donors. The ELISPOT assay may include isolating peripheral blood mononuclear cells (PBMC) from at least 10, preferably at least 40, subjects, often the subjects are human donors. The amount of at least one cytokine production in cells following administration of the peptides of putative epitopes may be determined by the ELISPOT assay. The at least one cytokine may be IFNγ and/or IL-10 such that data indicating cytokine production may represent a positive or a negative response of cells to the peptide sequence of the putative epitope. In some cases, positive responses may have a statistically significant difference (p<0.05) between a mean number of spots from five replicates in the experimental wells and the mean number from no antigen control wells. In other cases, negative responses may not have a statistically significant difference (p<0.05) between a mean number of spots from five replicates in the experimental wells and the mean number from no antigen control wells.

A ratio of the sub-types (e.g., Th1 and/or Th2) of immune responses may be calculated from the results of the immunological assay, often an ELISPOT assay. In some cases, a Th1/Th2 ratio may be calculated such that the magnitude and frequency of the immunological assay responses (e.g., ELISPOT) for each of the peptide sequences of the putative epitopes. For example, the ratio may be calculated using the following algorithm: (corrected mean spots per well)×(percent of responding subjects). In some cases, an activity ratio for each putative epitope may be determined. For example, the activity ratio may be calculated using the following algorithm: ((mean incidence of IFNγ×mean magnitude of IFNγ)/(mean incidence IL10×mean magnitude of IL-10)).

In some cases, the data obtained from performing at least the immunological assays are determined, compiled and results calculated using a digital processing device (e.g. computer) as described herein. For example, the data from the immunological assays may be analyzed and epitopes selected based on the output of the immunological assays. In some cases, the data obtained from performing at least the calculations with the algorithms are determined, compiled and results calculated using a computer as described herein. For example, the data from the algorithms may be analyzed and epitopes selected based on the output of the algorithms. In some cases, the data obtained from performing at least the immunological assays and the data obtained from performing at least the calculations with the algorithms are determined, compiled and results calculated using a computer as described herein. Often, the data from both the immunological assays and the algorithms may be analyzed and epitopes selected based on the outputs of both the immunological assays and the algorithms.

The methods or processes described herein may be used for designing compositions, often vaccines, comprising portions of peptides. In some cases, the portions of the peptides may be subunits of the peptides. For example, the compositions may contain nucleic acids encoding amino acid sequences of the portions of the peptides or of the subunits of the peptides. Using the methods described herein, the sub-type of the immune response elicited in a subject may be tuned by administering a portion of a peptide, a subunit of a peptide or an epitope of a peptide to a subject such that the portion of a peptide, a subunit of a peptide or an epitope of a peptide may elicit a desired sub-type of an immune response (e.g., Th1 or Th2) in a subject.

The methods or processes described herein may be used for designing compositions, often vaccines, comprising nucleic acids encoding portions of peptides. In some cases, the nucleic acids encoding portions of the peptides may be subunits of the peptides. For example, the compositions may contain nucleic acids encoding amino acid sequences of the portions of the peptides or of the subunits of the peptides. Using the methods or processes described herein, the sub-type of the immune response elicited in a subject may be tuned by administering a nucleic acids encoding portion of a peptide, a nucleic acids encoding a subunit of a peptide or nucleic acids encoding an epitope of a peptide to a subject such that the nucleic acids encoding a portion of a peptide, a nucleic acids encoding a subunit of a peptide or a nucleic acids encoding an epitope of a peptide may elicit a desired sub-type of an immune response (e.g., Th1 or Th2) in a subject.

In some cases, vaccines comprising portions of peptides, subunits of peptides or epitopes of peptides may be effective in preventing the onset or progression of a disease when administered to a subject. In other cases, vaccines comprising portions of peptides, subunits of peptides or epitopes of peptides may be more effective in preventing the onset or progression of a disease than a vaccine comprising a whole peptide or a whole peptide when administered to a subject. For example, the portion of a peptide, a subunit of a peptide or an epitope of a peptide may be more effective at eliciting a Th1 response in a subject compared to a whole peptide or a whole peptide, often the Th1 response may be desired in the subject compared to the Th2 response. For another example, the portion of a peptide, a subunit of a peptide or an epitope of a peptide may be more effective at eliciting a Th2 response in a subject compared to a whole peptide or a whole peptide, often the Th2 response may be desired in the subject compared to the Th1 response. Often, the methods or processes described herein may identify portions of peptides, subunits of peptides or epitopes of peptides that may be removed from a whole peptide or a whole peptide such that a desired sub-type of an immune response may be achieved in subject for prevention or elimination of a disease.

The methods or processes described herein may be used for designing compositions, often vaccines, comprising nucleic acids encoding portions of peptides. In some cases, the nucleic acids encoding portions of the peptides may be subunits of the peptides. For example, the compositions may contain nucleic acids encoding amino acid sequences of the portions of the peptides or of the subunits of the peptides. Using the methods or processes described herein, the sub-type of the immune response elicited in a subject may be tuned by administering a nucleic acids encoding portion of a peptide, a nucleic acids encoding a subunit of a peptide or nucleic acids encoding an epitope of a peptide to a subject such that the nucleic acids encoding a portion of a peptide, a nucleic acids encoding a subunit of a peptide or a nucleic acids encoding an epitope of a peptide may elicit a desired sub-type of an immune response (e.g., Th1 or Th2) in a subject.

In some cases, vaccines comprising nucleic acids encoding portions of peptides, nucleic acids encoding subunits of peptides or nucleic acids encoding epitopes of peptides may be effective in preventing the onset or progression of a disease when administered to a subject. In other cases, vaccines comprising nucleic acids encoding portions of peptides, nucleic acids encoding subunits of peptides or nucleic acids encoding epitopes of peptides may be more effective in preventing the onset or progression of a disease than a vaccine comprising a whole peptide or a whole peptide when administered to a subject. For example, nucleic acids encoding portion of a peptide, nucleic acids encoding a subunit of a peptide or nucleic acids encoding an epitope of a peptide may be more effective at eliciting a Th1 response in a subject compared to a whole peptide or a whole peptide, often the Th1 response may be desired in the subject compared to the Th2 response. For another example, nucleic acids encoding the portion of a peptide, nucleic acids encoding a subunit of a peptide or nucleic acids encoding an epitope of a peptide may be more effective at eliciting a Th2 response in a subject compared to a whole peptide or a whole peptide, often the Th2 response may be desired in the subject compared to the Th1 response. Often, the methods or processes described herein may identify nucleic acids encoding portions of peptides, nucleic acids encoding subunits of peptides or nucleic acids encoding epitopes of peptides that may be removed from a whole peptide or a whole peptide such that a desired sub-type of an immune response may be achieved in subject for prevention or elimination of a disease.

In some cases, each epitope within the set of putative epitopes may be differentiated by induction of a Type I immune response. In some cases, the sub-type of the immune response is a Type I immune response. In some cases, the sub-type of the immune response is a Type II immune response. In some cases, the Type I response is determined by measuring production of interferon gamma (IFNγ), interleukin-12 (IL-12), TNFα, or GM-CSF in the subject. In some cases, the Type II response is determined by measuring production of interleukin-10 (IL-10), interleukin-4 (IL-4), interleukin-5 (IL-5), or interleukin-6 (IL-6) in the subject. In some cases, each epitope within the set of putative epitopes may be differentiated by suppression of a Type I immune response. In some cases, each epitope within the set of putative epitopes may be differentiated by induction of a Type II immune response.

In some cases, the set of desired epitopes are presented on antigen presenting cells (APC)s in the subject. In some cases, the APCs in the subject are endogenous.

In some cases, arranging of the desired epitopes comprises separating two or more epitopes with a sequence of linker nucleic acids. In some cases, arranging of the desired epitopes comprises arranging two or more adjacent epitopes.

### Applications

The methods or processes described herein may identify desired epitopes from peptides such that the nucleic acids encoding the amino acids of the desired epitopes may be included in a composition administered to a subject to prevent or treat a disease, often a vaccine. In some cases, the vaccine may prevent or treat cancer. The methods or processes described herein may identify desired epitopes from peptides such that the amino acids of the desired epitopes may be included in a composition administered to a subject to prevent or treat a disease, often a vaccine to prevent or treat a disease. In some cases, the subject may be a subject in need of a vaccine. In some cases, the vaccine may be administered to a subject who does not have a disease. In other cases, the vaccine may be administered to a subject who has a disease.

In some cases, the subject may be a healthy individual. In some cases, the subject may be an individual with a disease. For example, the individual may be a patient. In some cases, the subject is a human individual. In other cases, the subject is a non-human individual. For example, non-human individuals may be a non-human primate, monkey, macaque, baboon, chimpanzee, orangutan, mouse, rat, guinea pig, rabbit, horse, cow, pig, dog, cat or any individual that may have had or has a disease.

Vaccine Construction.

The methods or processes described herein may identify at least one epitope of a peptide that may elicit at least one sub-type of an immune response in a subject. In some cases, a peptide-based vaccine may comprise at least one epitope of a peptide that may elicit an immune response in a subject. In other cases, a plasmid-based vaccine may comprise at least a nucleic acid sequence encoding an amino acid sequence of at least one epitope of a peptide that may elicit at least one sub-type of an immune response in a subject.

In some cases, a peptide-based vaccine may comprise at least one epitope of a peptide that may elicit an immune response in a subject. In other cases, a peptide-based vaccine may comprise at least a nucleic acid sequence encoding an amino acid sequence of at least one epitope of a peptide that may elicit at least one sub-type of an immune response in a subject.

For example, the peptide IGFBP-2 may elicit an immune response in a subject. In some cases, the peptides comprising two epitopes (e.g. IGFBP-2 (1-163) (N-terminus) and IGFBP-2 (164-328) (C-terminus)) may be identified to elicit at least one sub-type of an immune response in a subject. For example, a vaccine composition may comprise at least the two epitopes (e.g. IGFBP-2 (1-163) (N-terminus) and IGFBP-2 (164-328) (C-terminus)).

In some cases, nucleic acids encoding the amino acids comprising two epitopes (e.g. IGFBP-2 (1-163) (N-terminus) and IGFBP-2 (164-328) (C-terminus)) may be identified to elicit at least one sub-type of an immune response in a subject. For example, a vaccine composition may comprise at least the nucleic acids encoding the amino acid sequences of two epitopes (e.g. IGFBP-2 (1-163) (N-terminus) and IGFBP-2 (164-328) (C-terminus)). Using standard molecular biology techniques, the nucleic acids may be cloned into an expression vector, for example pUMVC3, the expression vector produced using standard molecular biology techniques and the sequence of the expression vector comprising the nucleic acids encoding the amino acid sequences of the at least one epitope. In some cases, cross-reactive sequences may be identified from sequences of either the N-terminal (amino acids 1-163), C-terminal IGFBP-2 (amino acids 164-328) or both. For example, sequences may be aligned with human, viral, bacterial or fungal peptides, often searching a database (e.g., ref_seq peptide in NCBI's DELTA-BLAST algorithm). Often, the default parameters for searching may be used. In some cases, alignments with less than 50% positivity (e.g., identical amino acids or conservative amino acid substitutions), less than 45% positivity, less than 40% positivity, less than 35% positivity, less than 30% positivity, less than 25% positivity or less than 20% positivity may be excluded from administration to a subject.

In some cases, expression of the at least one epitope peptide may be determined using standard biochemical techniques, for example, Western blot probing with an antibody which binds to the peptide epitope produced by the expression vector prior to administration of at least one vector to a subject.

The methods described herein include a method for designing a peptide vaccine, the method comprising: determining a potential of a set of putative epitopes to induce a sub-type of an immune response; ranking a plurality of putative epitopes from the set of putative epitopes by the sub-type of the immune response; from the plurality of putative epitopes ranked in step (b), identifying a set of desired epitopes such that the set of desired epitopes induces a desired sub-type of an immune response in a subject; and arranging the desired epitopes to provide a plasmid vaccine design.

The processes described herein include a peptide vaccine designed by the process of determining the potential of each putative epitope within the set of putative epitopes to induce a sub-type of an immune response; ranking a plurality of putative epitopes from the set of putative epitopes by the sub-type of the immune response; identifying a set of desired epitopes from the ranking such that the set of desired epitopes is capable of inducing a desired sub-type of an immune response in a subject; and designing a peptide vaccine from the set of desired epitope.

In some cases, arranging of the desired epitopes comprises separating two or more epitopes with a sequence of linker amino acids. In some cases, arranging of the desired epitopes comprises arranging two or more adjacent epitopes. In some cases, the desired sub-type of immune response is characterized by a ratio of Type I cytokine production to Type II cytokine production that is less than 1.

In some cases, the set of putative epitopes comprise a set of epitopes of self-proteins of the subject. In some cases, the set of putative epitopes contains epitopes from between about 2 and about 50 unique peptides. In some cases, the epitopes are extended epitopes. In some cases, the epitopes are derived from the same peptide. In some cases, the epitopes are derived from different peptides. In some cases, the epitopes are derived from the same antigen. In some cases, the epitopes are derived from different antigens.

In some cases, the epitopes may be derived from human proteins that may be used directly in a peptide based vaccine. In other cases, the epitopes may be derived from human proteins and the encoding nucleic acid sequences encoding the epitopes may be incorporated into a nucleic acid construct designed to induce expression of the epitope in a subject following administration. For example, epitopes encoded from the nucleic acid construct may allow for the immune response to at least one epitope to be entrained, amplified, attenuated, suppressed, or eliminated to specific sets of proteins (e.g., self-proteins). In some cases, the peptide or the nucleic acid construct may be optimized into a protein or plasmid-based vaccination to induce, amplify or entrain a Th1 immune response. In some cases, the epitopes may be extended Th1 epitopes. In other cases, the peptide or the nucleic acid construct may be optimized into a protein or plasmid-based vaccination to suppress, attenuate or eliminate a pathological response, in a subject (e.g., human or animal) in need thereof. In some cases, the set of putative epitopes is overexpressed in a subject with a disease compared to a subject without a disease.

In some cases, the method further comprises producing the plasmid vaccine, the plasmid vaccine comprising a set of nucleic acid sequences encoding a set of amino acids of the set of desired epitopes. In some cases, the method further comprises administering the plasmid vaccine to a subject.

### Vaccine Design Utilizing a System

The methods and processes described herein may further be carried out on a system. In some instances, the system is a system for designing a plasmid vaccine, which comprises a digital processing device comprising an operating system configured to perform executable instructions, and an electronic memory; a set of putative epitopes stored in the electronic memory; a computer program including instructions executable by the computer to create an application comprising: (i) a first software module configured to determine the potential of each putative epitope within the set of putative epitopes to induce a sub-type of an immune response; (ii) a second software module configured to rank a plurality of putative epitopes from the set of putative epitopes by the sub-type of the immune response, and identify a set of desired epitopes from the ranking, wherein the set of desired epitopes is capable of inducing a desired sub-type of an immune response in a subject; and (iii) a third software module configured to design a plasmid vaccine from the set of desired epitope identified in step (ii).

In some instances, the system is a system for designing a peptide vaccine, which comprises a digital processing device comprising an operating system configured to perform executable instructions, and an electronic memory; a set of putative epitopes stored in the electronic memory; a computer program including instructions executable by the computer to create an application comprising: (i) a first software module configured to determine the potential of each putative epitope within the set of putative epitopes to induce a sub-type of an immune response; (ii) a second software module configured to rank a plurality of putative epitopes from the set of putative epitopes by the sub-type of the immune response, and identify a set of desired epitopes from the ranking such that the set of desired epitopes is capable of inducing a desired sub-type of an immune response in a subject; and (iii) a third software module configured to design a peptide vaccine from the set of desired epitope identified in step (ii).

The system may further comprise a fourth software module that is configured to identify the set of putative epitopes from a literature search, a database search, a search of bioinformatics mediums, an analysis of a fluid sample from a subject, an analysis of a cellular sample from a subject, an analysis of a tissue sample from a subject, or a combination thereof.

The second software module may be further configured to rank the plurality of putative epitopes from the set of putative epitopes based on an adaptive immune response of the set of putative peptides in a subject. The second software module may be further configured to rank each putative epitope from the set of putative epitopes according to a parameter selected from: a binding of each epitope to major histocompatibility complex (MHC) alleles, an affinity of each epitope for major histocompatibility complex (MHC) alleles, or a combination thereof. Each epitope ranked in the top two quartiles of the set of putative epitopes may be identified in the set of desired epitopes.

The sub-type of the immune response may be selected from: production of IgG antibodies, production of specific Th cells in response to at least the first set of putative peptides, or a combination thereof. The sub-type of the immune response may be determined by an assay selected from: an enzyme linked immunosorbant assay (ELISA), an enzyme linked immunosorbant spot (ELISPOT) assay, a delayed type hypersensitivity responses (DTH), a lymphocyte proliferation or a cytoxicity assay, or a combination thereof.

The sub-type of the immune response may be a Type I immune response or a Type II immune response. The Type I response may be determined by an assay that measures the production of interferon gamma (IFNγ), interleukin-12 (IL-12), TNFα, or GM-CSF in the subject. The Type II response may be determined by an assay that measures the production of interleukin-10 (IL-10), interleukin-4 (IL-4), interleukin-5 (IL-5), or interleukin-6 (IL-6) in the subject. IFNγ may be measured using an assay selected from: ELISPOT assay, ELISA, rtPCR analysis of mRNA expression, immunohistochemistry, fluorescence in situ hybridization analysis (FISH), or a combination thereof. IL-10 may be measured using an assay selected from: ELISPOT assay, ELISA, rtPCR analysis of mRNA expression, immunohistochemistry, and fluorescence in situ hybridization analysis (FISH), or a combination thereof.

Each epitope within the set of putative epitopes may be differentiated by induction of a Type I immune response. Each epitope within the set of putative epitopes may be differentiated by suppression of a Type I immune response. Each epitope within the set of putative epitopes may be differentiated by induction of a Type II immune response.

Type II response may be determined by an assay that measures the production of interleukin-10 (IL-10), interleukin-4 (IL-4), interleukin-5 (IL-5), or interleukin-6 (IL-6) in the subject. IL-10 may be measured using an assay selected from: ELISPOT assay, ELISA, rtPCR analysis of mRNA expression, immunohistochemistry, and fluorescence in situ hybridization analysis (FISH), or a combination thereof.

Each epitope within the set of putative epitopes may be differentiated by induction of a Type I immune response. Each epitope within the set of putative epitopes may be differentiated by suppression of a Type I immune response. Each epitope within the set of putative epitopes may be differentiated by induction of a Type II immune response.

The set of desired epitopes may be presented on antigen presenting cells (APC)s in the subject. The APCs in the subject may be endogenous.

The arranging of the desired epitopes may comprise separating two or more epitopes with a sequence of linker nucleic acids. The arranging of the desired epitopes may comprise arranging two or more adjacent epitopes.

The subject may be a human. The human may have a disease. The human may be a healthy individual.

The computer may be connected to a computer network.

Digital Process Device.

In some instances, the digital process device may include one or more hardware central processing units (CPU) that carry out the device's functions. The digital processing device may further comprise an operating system configured to perform executable instructions. The digital processing device may be optionally connected to a computer network, the Internet such that it accesses the World Wide Web, a cloud computing infrastructure, an intranet, or a data storage device.

Suitable digital processing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers include those with booklet, slate, and convertible configurations, known to those of skill in the art.

The digital processing device may include an operating system configured to perform executable instructions. The operating system may, for example, include programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some instances, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, GoogleTV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

The device may include a storage and/or memory device. The storage and/or memory device may be one or more physical apparatuses used to store data or programs on a temporary or permanent basis. In some instances, the device is volatile memory and requires power to maintain stored information. In other instances, the device is non-volatile memory and retains stored information when the digital processing device is not powered. The non-volatile memory may comprise flash memory. The non-volatile memory may comprise dynamic random-access memory (DRAM). The non-volatile memory may comprise ferroelectric random access memory (FRAM). The non-volatile memory may comprise phase-change random access memory (PRAM). The device may be a storage device including, by way of non-limiting examples, CD-ROMs, DVDs, flash memory devices, magnetic disk drives, magnetic tapes drives, optical disk drives, and cloud computing based storage. The storage and/or memory device may be a combination of devices such as those disclosed herein.

The digital processing device may include a display to send visual information to a user. In some instances, the display is a cathode ray tube (CRT), a liquid crystal display (LCD), a thin film transistor liquid crystal display (TFT-LCD), or an organic light emitting diode (OLED) display. An OLED display may be a passive-matrix OLED (PMOLED) or active-matrix OLED (AMOLED) display. The display may be a plasma display. The display may be a video projector. The display may be a combination of devices such as those disclosed herein.

The digital processing device may include an input device to receive information from a user. The input device may be a keyboard. The input device may be a pointing device including, by way of non-limiting examples, a mouse, trackball, track pad, joystick, game controller, or stylus. The input device may be a touch screen or a multi-touch screen. The input device may be a microphone to capture voice or other sound input. The input device may be a video camera or other sensor to capture motion or visual input. The input device may be a Kinect™, Leap Motion™, or the like. The input device may be a combination of devices such as those disclosed herein.

Non-Transitory Computer Readable Storage Medium.

The systems, methods, and processes disclosed herein may include one or more non-transitory computer readable storage media encoded with a program including instructions executable by the operating system of an optionally networked digital processing device. A computer readable storage medium may be a tangible component of a digital processing device. A computer readable storage medium may be optionally removable from a digital processing device. A computer readable storage medium may include, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Computer Program.

The systems, methods, and processes disclosed herein may include at least one computer program, or use of the same. A computer program may include a sequence of instructions, executable in the digital processing device's CPU, written to perform a specified task. In some instances, computer readable instructions are implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain cases, is written in various versions of various languages.

In some instances, the functionality of the computer readable instructions are combined or distributed as desired in various environments. A computer program may comprise one sequence of instructions. A computer program may comprise a plurality of sequences of instructions. A computer program may be provided from one location. A computer program may be provided from a plurality of locations. A computer program may include one or more software modules. A computer program may include, in part or in whole, one or more web applications, one or more mobile applications, one or more standalone applications, one or more web browser plug-ins, extensions, add-ins, or add-ons, or combinations thereof.

Web Application.

A computer program may include a web application. In light of the disclosure provided herein, those of skill in the art will recognize that a web application, in various instances, utilizes one or more software frameworks and one or more database systems. A web application may be created upon a software framework such as Microsoft® .NET or Ruby on Rails (RoR). A web application may utilize one or more database systems including, by way of non-limiting examples, relational, non-relational, object oriented, associative, and XML database systems. Suitable relational database systems may include, by way of non-limiting examples, Microsoft® SQL Server, mySQL™, and Oracle®. A web application may be written in one or more markup languages, presentation definition languages, client-side scripting languages, server-side coding languages, database query languages, or combinations thereof. A web application may be written to some extent in a markup language such as Hypertext Markup Language (HTML), Extensible Hypertext Markup Language (XHTML), or eXtensible Markup Language (XML). A web application may be written to some extent in a presentation definition language such as Cascading Style Sheets (CSS). A web application may be written to some extent in a client-side scripting language such as Asynchronous Javascript and XML (AJAX), Flash® Actionscript, Javascript, or Silverlight®. A web application may be written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. A web application may be written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

Mobile Application.

A computer program may include a mobile application provided to a mobile digital processing device. The mobile application may be provided to a mobile digital processing device at the time it is manufactured. The mobile application may be provided to a mobile digital processing device via the computer network described herein.

A mobile application may be created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments may be available from several sources. Commercially available development environments may include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers may distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums may be available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Application.

A computer program may include a standalone application, which is a program that is run as an independent computer process, not an add-on to an existing process, e.g., not a plug-in. Those of skill in the art will recognize that standalone applications are often compiled. A compiler may be a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation may be often performed, at least in part, to create an executable program. In some aspects, a computer program includes one or more executable complied applications.

Web Browser Plug-in.

The computer program may include a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some instances, the toolbar comprises one or more web browser extensions, add-ins, or add-ons. In some cases, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) may be software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some instances, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules.

The systems, methods, and processes disclosed herein may include software, server, and/or database modules, or use of the same. In view of the disclosure provided herein, software modules are created by techniques known to those of skill in the art using machines, software, and languages known to the art. The software modules disclosed herein may be implemented in a multitude of ways. A software module may comprise a file, a section of code, a programming object, a programming structure, or combinations thereof. A software module may comprise a plurality of files, a plurality of sections of code, a plurality of programming objects, a plurality of programming structures, or combinations thereof. The one or more software modules may comprise, by way of non-limiting examples, a web application, a mobile application, and a standalone application. In some instances, software modules are in one computer program or application. In other instances, software modules are in more than one computer program or application. In some cases, software modules are hosted on one machine. In other cases, software modules are hosted on more than one machine. In further cases, software modules are hosted on cloud computing platforms. In some aspects, software modules are hosted on one or more machines in one location. In other aspects, software modules are hosted on one or more machines in more than one location.

Databases.

The methods, systems, and processes disclosed herein may include one or more databases, or use of the same. In view of the disclosure provided herein, those of skill in the art will recognize that many databases may be suitable for storage and retrieval of analytical information described elsewhere herein. Suitable databases may include, by way of non-limiting examples, relational databases, non-relational databases, object oriented databases, object databases, entity-relationship model databases, associative databases, and XML databases. A database may be internet-based. A database may be web-based. A database may be cloud computing-based. A database may be based on one or more local computer storage devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Identification of Unique Amino Acid Sequences From Endoglin (CD105) and Hypoxia Induced Factor (HIF)-1α for a Vaccine

This example describes the identification of unique and discrete amino acid sequences of antigenic epitopes from breast cancer stem cell and epithelial mesenchymal transformation peptides CD105 and HIF-1a, both of which are promiscuous MHC class II binders and unexpectedly stimulate high (magnitude and incidence) interferon gamma (IFNγ) and low or no interleukin (IL)-10 responses from human peripheral blood mononuclear cells (PBMC). CD105 and HIF1α have been juxtaposed to allow construction of extended epitope sequences for inclusion in either a peptide or DNA plasmid-based multi-antigen polyepitope CD4+ T-cell vaccine targeting breast cancer stem cell and epithelial mesenchymal transformation antigens in cancer patients.

Three peptides (p 96-114, p116-130 and p214-236) in the N terminal region of CD105 and one at the C-terminus (p626-642) stimulated strong IFNγ, low or no IL-10 responses, with IFNγ response incidence of 20% or greater. Three peptides at the N terminus of HIF1α (p38-53, p60-82, and p 93-117) stimulated strong IFNγ and no or low IL-10 responses. These peptides were identified via the stepwise screening method described herein.

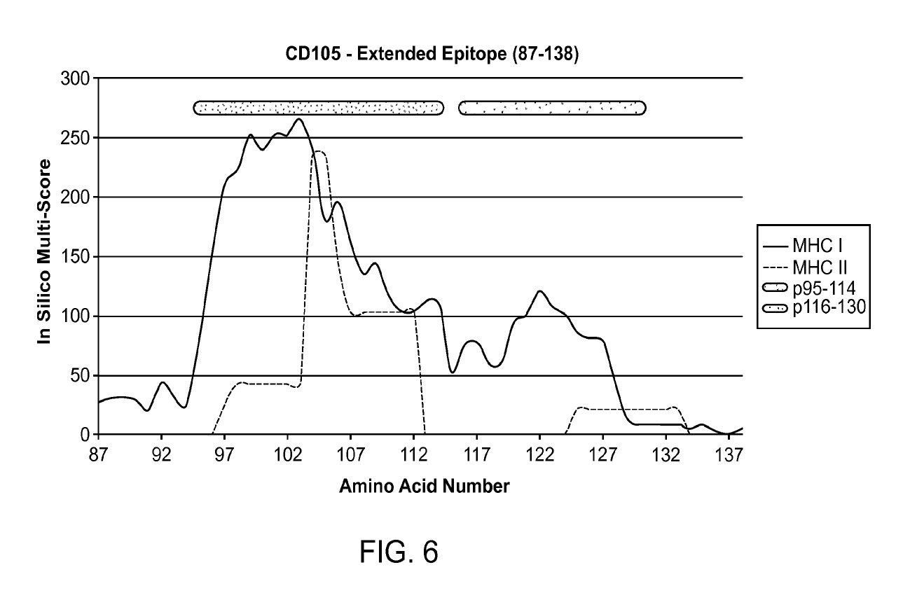

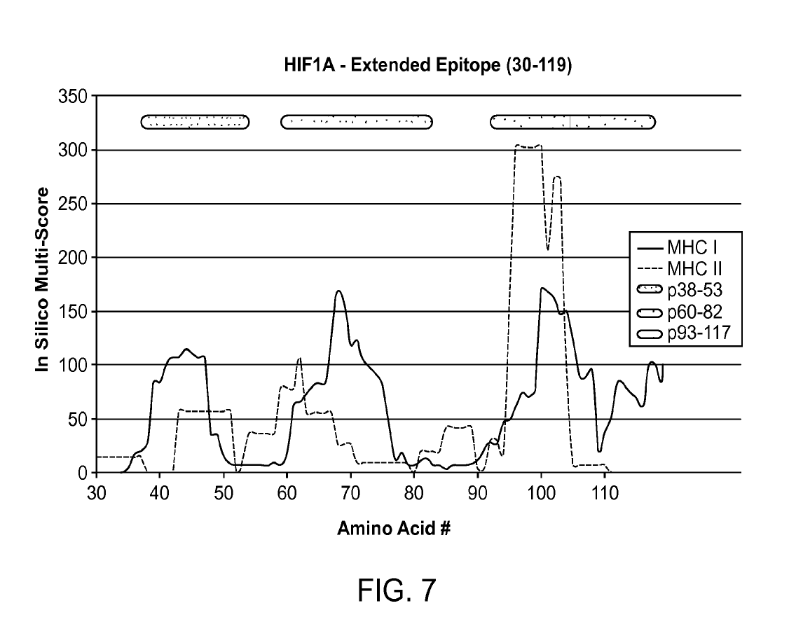

The CD105 Extended Epitope Amino acid sequence =
(SEQ ID NO: 1)
QNGTWPREVLLVLSVNSSVFLHLQALGIPLHLAYNSSLVTFQEPPGVNT
TEL Length = 52 amino acids MW = 5.7 kDa
(see FIG. 6).
HIF1α Extended Epitope Amino acid sequence =
(SEQ ID NO: 2)
RSKESEVFYELAHQLPLPHNVSSHLDKASVMRLTISYLRVRKLLDAGDL
DIEDDMKAQMNCFYLKALDGFVMVLTDDGDMIYISDNVNKY
Length = 90 aa MW = 10.4 kDa (see FIG. 7).

This example demonstrates that epitopes of CD105 and HIF1α may be singly for short epitopes (15-20-mer) or may be juxtaposed so as to allow the design of extended epitope vaccines (40-80-mer peptides) as described herein The close proximal juxtaposition (within 10 amino acids of each other) of certain of these selected peptides within the parent peptide may allow construction of in-tandem extended epitopes that are unlikely to contain (within the relatively short intervening, <10 amino acid sequences) tolerating and/or suppressive epitopes.

TABLE 1
Amino Acid Sequences of Peptides

| | | |
|---|---|---|
| HIF-1a p38-53 | YELAHQLPLPHNVSSH (16 amino acids) | SEQ ID NO: 3 |
| HIF-1a p60-82 | MRLTISYLRVRKLLDAGDLDIED (23 amino acids) | SEQ ID NO: 4 |
| HIF-1 a 93-117 | LKALDGFVMVLTDDGDMIYISDNVN (25 amino acids) | SEQ ID NO: 5 |
| CD105 p 96-114 | VLLVLSVNSSVFLHLQALGI (20 amino acids) | SEQ ID NO: 6 |
| CD105 p116-130 | LHLAYNSSLVTFQEP (15 amino acids) | SEQ ID NO: 7 |
| CD105 p214-236 | GHKEAHILRVLPGHSAGPRTVTV (23 amino acids) | SEQ ID NO: 8 |

Using the methods described herein, as well as in silico based epitope prediction analysis (for promiscuous binding to MHC class II), an extended epitope plasmid and short epitope plasmids from both CD105 (p87-138) and HIF1α (p30-119) may be constructed. Any of the peptides listed in Table 1 above, and/or extended epitopes (either as the peptide itself, or as the corresponding nucleic acid construct) singly, or in any combination, may be optimized into a peptide or plasmid-based vaccination that may specifically induce, amplify or entrain a protective immune response, or alternatively, will suppress, attenuate or eliminate a pathological one, in a subject (human or animal) in need thereof.

### Example 2

### Elimination of IL-10 Inducing T-Helper Epitopes from an IGFBP-2 Vaccine Ensures Potent Anti-Tumor Activity

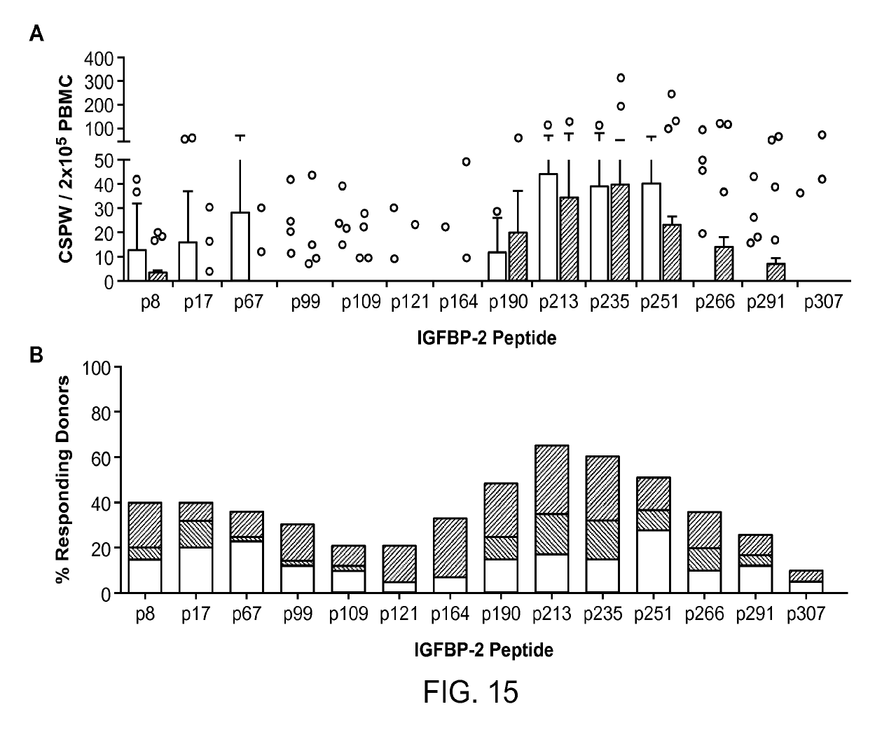

This example describes the identification of unique and discrete amino acid sequences of antigenic epitopes from IGFBP-2 and elimination of amino acid sequences which induce IL-10 secretion in a subject following administration (e.g., immunization) of the epitopes. Immunization against self-tumor antigens can induce T-regulatory cells which inhibit proliferation of Type I CD4+T-helper (Th1) and CD8+ cytotoxic T-cells. Type I T-cells are required for potent anti-tumor immunity. Immunosuppressive epitopes were identified and deleted from a cancer vaccine targeting IGFBP-2 to enhance vaccine efficacy. Epitopes in the N-terminus of IGFBP-2 that elicited predominantly Th1 while the C-terminus stimulated Th2 and mixed Th1/Th2 responses were identified by screening breast cancer patient lymphocytes with IFNγ and IL-10 ELISPOT (see FIG. 15). Epitope-specific Th2 responses demonstrated a higher functional avidity for antigen than epitopes which induced IFNγ (p=0.014).

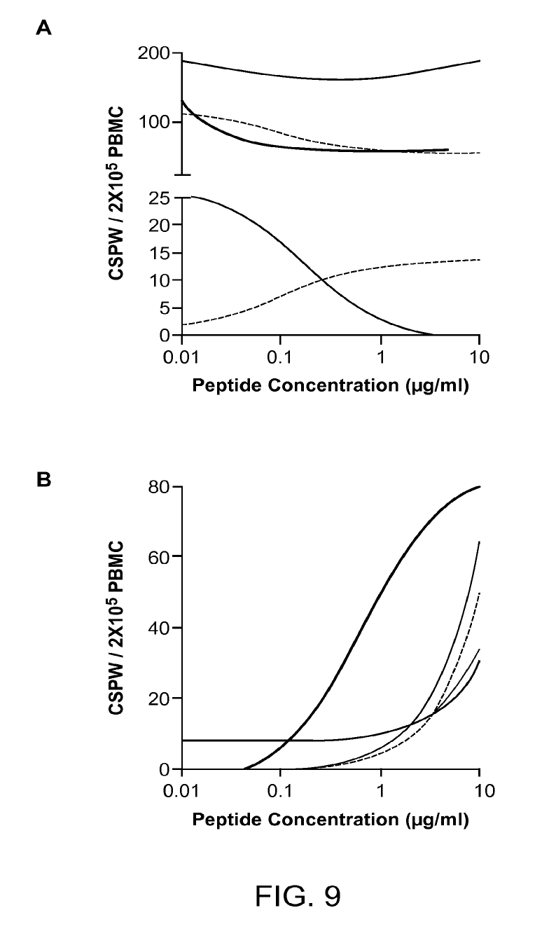

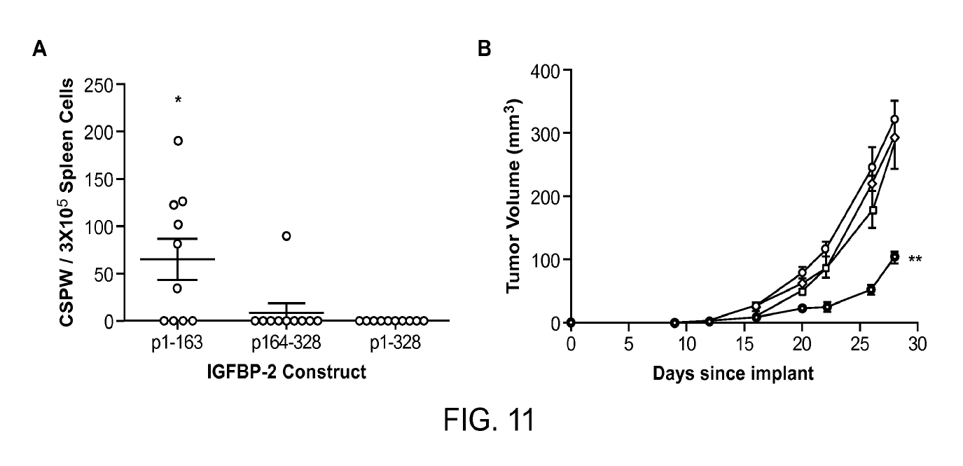

TgMMTV-neu mice were immunized with DNA constructs encoding IGFBP-2 N- and C-termini. T-cell lines expanded from the C-terminus vaccinated animals secreted significantly more Type II cytokines than those vaccinated with the N-terminus and could not control tumor growth when infused into tumor-bearing animals (see FIG. 9). In contrast, N-terminus epitope-specific T-cells secreted Th1 cytokines and significantly inhibited tumor growth, as compared with naïve T-cells, when adoptively transferred (p=0.005) (see FIG. 11).

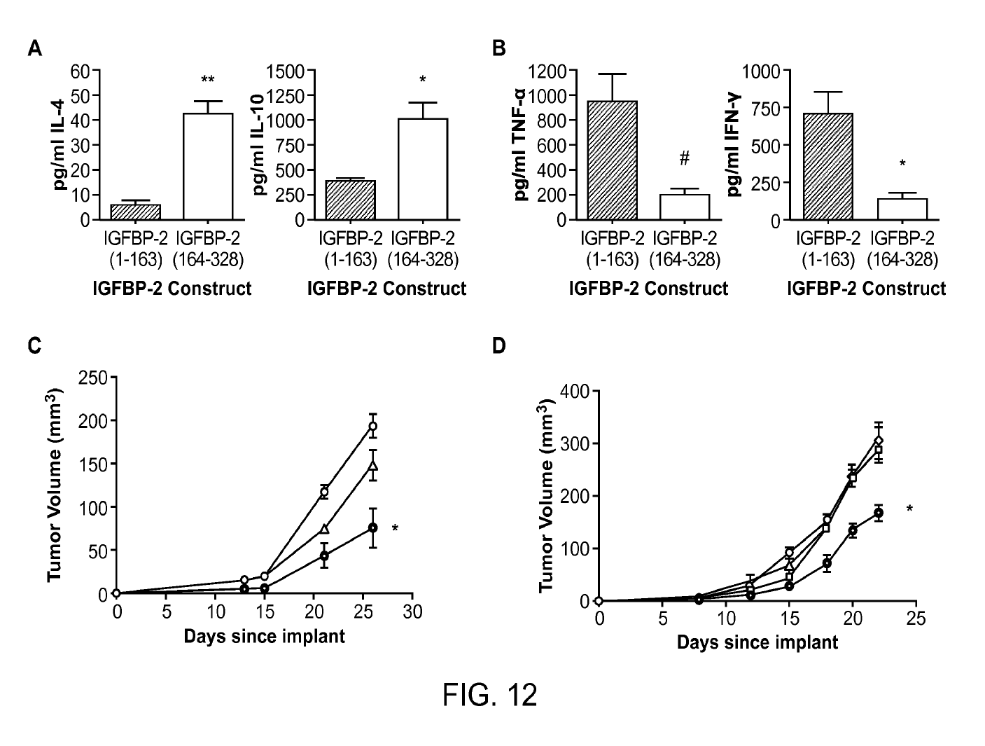

To determine whether removal of Th2 inducing epitopes had any effect on the vaccinated anti-tumor response, mice were immunized with the N-terminus, C-terminus and a mix of equivalent concentrations of both vaccines. The N-terminus vaccine significantly inhibited tumor growth (p<0.001) as compared to the C-terminus vaccine which had no anti-tumor effect. Mixing the C-terminus with the N-terminus vaccine abrogated the anti-tumor response of the N-terminus vaccine alone (see FIG. 12).

Epitopes derived from a self-tumor antigen were screened for sequences that may induce antigen-specific Treg or Th2. Those sequences were then eliminated from those epitopes to enhance vaccine efficacy. Specific peptides of self-peptides preferentially elicit IFNγ or IL-10 secretion by T-cells. Elimination of epitopes that elicit IL-10 secretion assures the anti-tumor potency of an IGFBP-2 directed vaccine.

Evaluation of Antigen-Specific T-Cell Phenotype and Functional Avidity.

IGFBP-2 peptides, predicted to bind promiscuously to human MHCII, were selected using web-based algorithms according to the methods described herein. Peripheral blood mononuclear cells (PBMC) from 20 female breast cancer patients were cryopreserved and evaluated by ELISPOT for antigen specific IFNγ or IL-10 secretion, according to our published methods. Some donors, who demonstrated either an IFNγ restricted (n=5) or IL-10 restricted epitope-specific response (n=5), had T-cells assessed at varying epitope concentrations; 10 μg/ml, 1 μg/ml, 0.1 μg/ml and 0.01 μg/ml for the appropriate cytokine secretion. Data is reported as the mean number of spots for each experimental antigen minus the mean number of spots detected in no antigen control wells (corrected spots per well: CSPW). Positive responses were defined by a statistically significant difference (p<0.05) between the mean number of spots from five replicates in the experimental wells and the mean number from no antigen control wells for an individual (see FIG. 15).

Antigen-specific IFNγ production by mouse spleen cells was quantitated by ELISPOT using PVDF plates (Millipore) that were coated with 10 μg/ml anti-mouse IFNγ (clone AN-18; Mabtech) and 5 μg/ml biotinylated anti-mouse IFNγ (clone R4-6A2; Mabtech). Data are reported as CSPW as defined above (see FIG. 11).

Generation of Human and Murine IGFBP-2-Specific Th1 and Th2 Cell Lines.

Human T-cell lines were generated using methods known to those of skill in the art. Spleen cells from IGFBP-2 (1-163) (N-terminus)-vaccinated mice were stimulated with a pool of peptides; p8-22, p17-31, p67-81, p99-113, p109-123 and p121-135 (10 μg/ml each) and IGFBP-2 (164-328) (C-terminus)-vaccinated mice were stimulated with p164-178, p190-204, p213-227, p235-249, p251-265, p266-280, p291-305, p307-321 (e.g., 10 μg/ml each) peptides. The T-cells were subjected to a second in vitro stimulation on day 8 by adding equivalent numbers of peptide-loaded (10 μg/ml) autologous irradiated (e.g., 3000 rads) splenic cells to the original culture. 10 ng/ml recombinant mouse IL-7 (e.g., R & D Systems), 5 ng/ml recombinant human IL-15 (e.g., PreproTech, Inc.) and 10 U/ml recombinant human IL-2 (e.g., Hoffman-LaRoche) were added on days 5 and 12, with additional IL-2 on days 15 and 18 for T-cell expansion.

Assessment of T-Cell Phenotype.

Receptor expression was documented in the expanded T-cells by adding PE-Cy7-conjugated anti-mouse CD49b (e.g., 0.5 μg), APC-conjugated anti-mouse CD4 (e.g., 0.2 μg) or APC-conjugated anti-human CD4 (e.g., 20 μl), PerCP-conjugated anti-mouse CD3 (e.g., 0.2 μg) or PE-Cy7-conjugated anti-human CD3 (e.g., 20 μl) and FITC-conjugated anti-mouse CD19 (e.g., 0.5 μg). For extracellular staining, cells were incubated 30 minutes with the receptor antibodies. Intracellular expression of FOXP3 was documented after permeablization and fixation with the FOXP3 Buffer Set (e.g., Biolegend) according to the manufacturer's instructions and staining with PE-conjugated anti-mouse FOXP3 (e.g., 0.5 μg) and anti-mouse CD4 or PE-conjugated anti-human FOXP3 (e.g., 20 μl) and anti-human CD4. Flow cytometry was performed on the FACSCanto (e.g., BD Biosciences) and data analyzed using FlowJo software (e.g., BD Biosciences). Typically, 100,000 cells were collected per sample. Results are reported as a percentage of total cell number or a percentage of a specific cell population.

Cytokine levels in the murine T-cell cultures were assessed according to manufacturer's instructions using the appropriate ELISA (e.g., eBioscience) on medium collected from the splenic T-cell lines on day 10 of culture.

Vaccine Construction.

IGFBP-2 (1-163) (N-terminus) and IGFBP-2 (164-328) (C-terminus) were amplified using the primers and conditions listed in Table 2 with the Herculase II polymerase (e.g., Stratagene). For the IGFBP-2 (1-328) (full length) construct, cDNA was made from RNA extracted from the human breast cancer cell line, MCF-7 (e.g., ATCC). The cDNA was amplified using primers and conditions listed in Table 2. The insert and eukaryotic expression vector, pUMVC3 (e.g., National Gene Vector Biorepository), were cut with EcoRl and BamHl restriction enzymes and ligated using E. coli ligase (e.g., New England Biolabs). Transformation of XL1 Blue competent bacteria (e.g., Stratagene) allowed kanamycin resistant clone selection. Sequencing (e.g., www.agencourt.com) was performed on each clone and each large scale DNA prep (e.g., Qiagen) to confirm identity. All DNA plasmids were determined to express the correct sized peptide in vitro by transfecting HEK-293 (e.g., ATCC) cells using Polyfect reagent (e.g., Qiagen) and Western blot probing with anti-IGFBP-2 polyclonal antibodies (e.g., Santa Cruz Biotechnology, Inc).

Sequence Alignment.

N-terminal (amino acids 1-163) or C-terminal IGFBP-2 (amino acids 164-328) sequences were aligned with human, viral, bacterial or fungal peptides searching the ref seq_peptide database in NCBI's DELTA-BLAST algorithm using the default parameters. Alignments with less than 35% positivity (identical amino acids or conservative amino acid substitutions) over 80 amino acids were excluded as insignificant homology. Thirty-five percent represents a conservative assessment for identification of potential cross-reactive sequences for allergens.

Vaccination, Adoptive Transfer, and Assessment of Tumor Growth.

Animal care and use were in accordance with institutional guidelines. Female FVB/N-TgN (MMTVneu)-202Mul mice (Tg-MMTVneu) (6-8 weeks old; mean weight: 18.5 g, range: 15.4-23.1 g) (e.g., Jackson Laboratory) were immunized with IGFBP-2 DNA constructs or pUMVC3 vector alone (e.g., 50 μg plasmid) as a mixture in complete Freund's adjuvant/incomplete Freund's adjuvant (e.g., Sigma). Three immunizations were given two weeks apart. For tumor challenge, a syngeneic mouse mammary tumor cell line, MMC, (e.g., 0.5×10⁶ cells) was implanted into the mammary fat pad two weeks after the last vaccine or seven days before T-cell adoptive transfer (n=10/group). Tumors were measured as previously described. All tumor growth is presented as mean tumor volume (mm³±SEM). Data are representative of two independent experiments. (see Tables 2-5).

For adoptive transfer, 5×10⁶ IGFBP-2 N-terminal- or C-terminal-specific T-cells were transferred into tumor-bearing mice by i.v. tail vein injection. The same number of splenocytes derived from naïve mice were used as a control infusion.

Statistical Analysis.

The unpaired, two-tailed Student's t-test, Fischer's exact test or X² test was used to evaluate differences between groups. The half maximal concentration (EC50) of peptide was calculated as log(agonist) vs. response and reported as mean±SEM for five donors with IFNγ and four donors for IL-10 responses (one IL-10 donor demonstrated no dose tritration at the concentrations evaluated). p<0.05 was considered significant. All statistical analyses were performed using GraphPad Prism 5.04 (e.g., GraphPad Software).

The IGFBP-2 C-Terminus is Enriched for Epitopes that Induce IL-10-Secreting T-Cells.

Investigations indicate the predominant cellular immune response in most patients with breast cancer is of a Th2 phenotype. Sequences within a self-antigen that were specific for eliciting Th1 vs. Th2 or Treg for the purpose of excluding immune suppressive sequences from an epitope-based vaccine construct were identified. Th2/Treg were analyzed by examining IL-10 secretion.

IGFBP-2 epitope-induced IL-10 and IFNγ secretion was variable in breast cancer PBMC. Epitopes within the C-terminus (p190-p307) of the peptide were more immunogenic, stimulating a greater magnitude IL-10 and IFNγ response than epitopes in the N-terminus. The mean IL-10 epitope-specific response (18 CSPW; range, 0-129 CSPW) in the C-terminus was 6-fold greater than the mean IL-10 epitope-specific response in the N-terminus (3 CSPW; range, 0-44 CSPW; p<0.001). The mean IFN-γ epitope-specific response (12 CSPW; range, 0-82 CSPW) in the C-terminus was 2-fold greater than the mean IFNγ epitope-specific response in the N-terminus (6 CSPW; range, 0-70 CSPW; p=0.022). Epitopes in the C-terminus equally elicited IFNγ and IL-10 secretion (p=0.132). In contrast, epitopes derived from the N-terminus of IGFBP-2 induced 3-fold more IFNγ secretion than IL-10 secretion (p=0.012) (see FIG. 15).

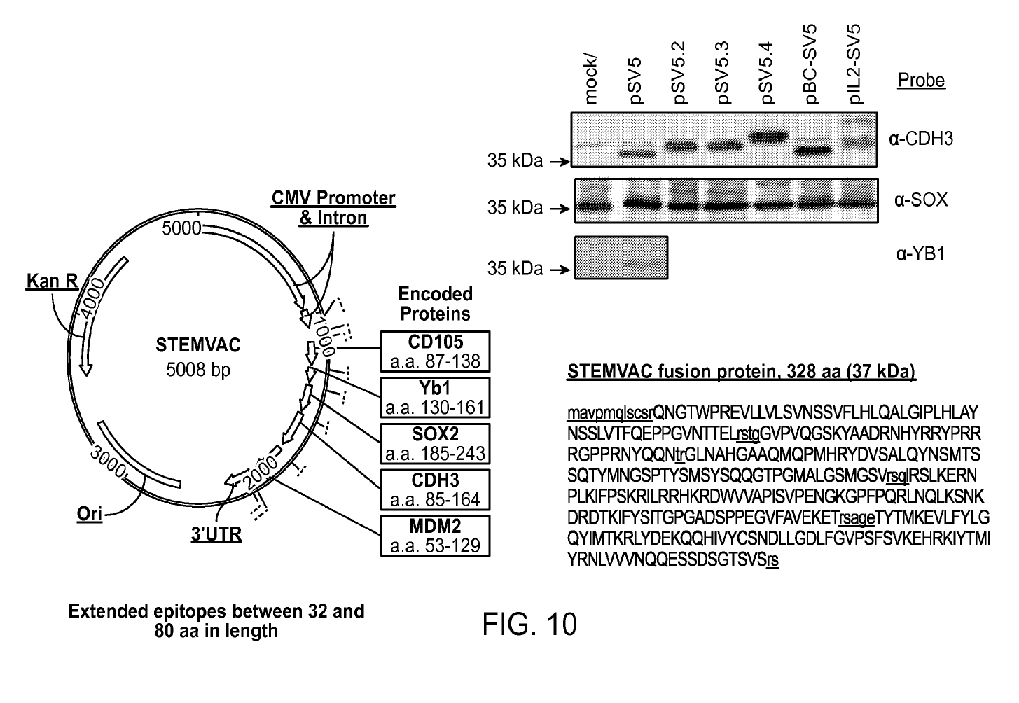

Individual epitopes were shown to induce exclusively IFNγ or IL-10 or the secretion of both cytokines (mixed) in the breast cancer patient population (FIG. 10). A significantly greater number of patients responded to the C-terminus of the peptide (mean responder, 42%), compared to the N-terminus (mean responder, 31%; p=0.007). The C-terminal epitopes induced a mix of both IL-10 and IFNγ secretion in response to antigen in a higher percentage of patients (mean responder, 20%) than that induced by the N-terminal epitopes (mean responder, 7%; p=0.003) where responses appeared to be more restricted to either Th1 or Th2.

To assess whether the T-cells elicited were Th2 or FOXP3+ Treg, the phenotype of cultured T-cell lines was evaluated. T-cells generated were CD3+(mean: 90%, range 86-94%) composed primarily of CD4+(mean: 73%, range: 70-77%) with fewer CD8+(mean: 27%, range: 24-30%). No culture demonstrated an outgrowth of Treg (mean CD4+ FOXP3+: 1.7%, range: 0.9-2.5%) as compared to baseline.

IGFBP-2 Epitope-Specific Th2 Demonstrate a Higher Functional Avidity and Homology to a Greater Number of Bacterial and Self-Peptides than IGFBP-2 Epitope-Specific Th1.

Titration studies documented that the peptides that induced IL-10 secretion were recognized by T-cells with a higher functional avidity (mean EC₅₀ concentration: 0.12±0.02 μg/ml) (FIG. 9) than those peptides that induced an IFNγ response (mean EC₅₀ concentration 2.1±0.43 μg/ml; p=0.014) (FIG. 9). The N- and C-terminus differed in the amount of sequence homologies shared with foreign antigens. 157 bacterial species that demonstrated 35% shared amino acid positivity over 80 or more amino acids (range 35-43%) for the human IGFBP-2 C-terminus were identified (see Table 3). In contrast, the N-terminus demonstrated no sequence homology with bacterial peptides, a difference of over 100-fold. There was no difference in the number of viral homologies between the two termini (N-term, 0 and C-term, 0).

The IGFBP-2 N-terminus shared significant homology with other IGFBP peptides, and only one additional self-peptide, CYR61 (see Table 4). The C-terminus also demonstrated significant homology with other IGFBP peptides but also to nine additional self-peptides including thyroglobulin, nidogens, and testicans (see Table 5). Only 16% of all homologous sequences for the N-terminus were non-IGFBP related while 64% of homologous sequences for the C-terminus were self-peptides other than IGFBP family members.

An N-Terminus, but not IGFBP-2 C-Terminus, Vaccine Both Stimulates Type I Immunity and Inhibits Tumor Growth.

Human and murine IGFBP-2 are highly homologous (82%) and tumors that arise in the TgMMTV-neu overexpress IGFBP-2. Mice were immunized with DNA constructs encoding the N-terminus (1-163), the C-terminus (164-328) and the full length (1-328) of IGFBP-2. The N-terminus vaccine could elicit peptide-specific Th1 (mean, 73 CSPW; range, 0-190 CSPW) compared to the C-terminus vaccine (mean, 10 CSPW; range, 0-89 CSPW; p=0.023) or the IGFBP-2 full length sequence (mean, 0 CSPW; p=0.007) (FIG. 11). The mean tumor volume of N-terminus vaccinated mice (104.2±8.4 mm³) was significantly less than that observed in the empty vector control (319.1±33.2 mm³), C-terminus immunized (295.8±15.5 mm³) and IGFBP-2 full length (278.3±33 mm³) vaccinated mice, (p<0.001 for all) (FIG. 11). Indeed, tumor growth after vaccination with the C-terminus and full length constructs was no different than control (p>0.15 for all).

IGFBP-2 Vaccine-Induced Th2 can Abrogate the Anti-Tumor Effect of IFGBP-2-Specific Th1.

Cytokine secretion was determined from T-cell lines generated after vaccination. T-cell lines derived from mice vaccinated with the N-terminus (mean, 77% CD3+ cells) were divided equally between CD4+(mean, 50%) and CD8+(mean, 50%) cells. T-cell lines generated from mice vaccinated with the C-terminus (mean, 65% CD3+ cells) were predominantly CD4+(mean, 64%) with fewer CD8+(mean, 36%) cells. Less than 0.5% of B cells, NK cells or FOXP3+CD4+ T-cells were detected in any culture. Expanded T-cell lines from the C-terminus secreted significantly more of the Type II cytokines IL-4 (mean, 42.4±5.4 ng/ml; p<0.001) and IL-10 (mean, 1011±154 ng/ml; p=0.002) than those from the N-terminus (mean IL-4, 5.5±1.9 ng/ml; mean IL-10, 368.8±45.5 ng/ml) (FIG. 12). T-cell lines from mice vaccinated with the N-terminus construct secreted significantly more Th1 cytokines, IFNγ (mean, 702.5±125.7 ng/ml; p=0.008) and TNFα (mean, 926±244 ng/ml; p=0.015) than T-cells from mice vaccinated with the C-terminus construct (mean IFNγ, 135.8±33.4 ng/ml; mean TNFα, 186.5±64.4 ng/ml) (FIG. 12). T-cells from mice vaccinated with the N-terminus adoptively transferred into tumor-bearing mice inhibited tumor growth (mean, 76.1±23.6 mm³) compared to naïve T-cells (mean, 195±14.4 mm³; p=0.005) (FIG. 12). Conversely, tumor growth in mice treated with T-cells derived from animals vaccinated with the C-terminus construct (mean, 149.2±18.3 mm³) was not statistically different than the naïve T-cell treated mice (p=0.09). Immunization with a vaccine which mixed both N- and C-terminus constructs in equivalent amounts abrogated the anti-tumor effect (mean, 292.3±16.7 mm³) of the N-terminus construct when used alone (mean, 178.7±16.6 mm³; p=0.001). Mean tumor growth after immunization with the combination vaccine was not significantly different than the empty vector control (313±41.3 mm³; p=0.712) or the C-terminus vaccine (164-328) alone (300.4±23.4 mm³; p=0.409) (FIG. 12).

The generation of tumor-specific Th1, via vaccination, can result in the activation of both innate immune cells and CD8+ cytotoxic T-cells (CTL). Vaccine-stimulated antigen-specific Th1 secrete Type I cytokines, such as IFNγ, which enhance the function of local APC and augment endogenous antigen presentation. An increased processing of tumor cells by the APC results in epitope spreading, which is associated with tissue destruction. Many current cancer vaccine approaches, especially those which employ the use of whole intact antigen, elicit Th2 or mixed Th1/Th2 immunity. Subunit or epitope-based vaccines may be much more effective for preferentially inducing Th1 than whole antigen approaches. Data presented here demonstrate that a self-tumor antigen contains sequences that are capable of specifically stimulating either a Th1 or Th2 response. Moreover, the Th2 generated by such epitopes are of a higher functional avidity than the Th1 cells elicited, thus may compete more effectively for antigen/MHC complexes at the site of the tumor. Removal of Th2 inducing sequences from a vaccine construct, however, will allow Th1 dominance and an effective anti-tumor response.

The differentiation of a naïve Th-cell into one with a mature phenotype is influenced by the binding of a particular peptide to the MHC (signal 1), the co-stimulation provided at the time of antigen recognition (signal 2), and the cytokine environment in which the immune response is generated (signal 3). Signals 2 and 3 can be influenced by the adjuvants provided with vaccination. Signal 1 was identified by determining whether immunosuppressive epitopes could be identified within a tumor antigen peptide sequence then removed. IGFBP-2 sequences stimulated both IFNγ as well as IL-10. Sequences that elicited predominantly IFNγ secretion in response to antigen, allowing epitopes that generated mixed responses were identified and removed from the vaccine construct.

The IFGBP-2 N- and C-termini differed significantly in the prevalence of Th1- vs. Th2-inducing epitopes. Using techniques known to those of skill in the art, methodology for predicting the potential for cross-reactivity to allergens which requires a minimum of 35% identity over 80 amino acid sequences to define risk for cross-interaction was used. The IGFBP-2 C-terminus harbored over 100-fold greater sequences with potential cross-reactivity to bacterial antigens than the N-terminus. The C-terminus had a greater sequence homology with numerous self-peptides outside of the insulin like growth factor receptor family, in contrast to the N-terminus whose homology was restricted.

TABLE 2
Primer Sequences and PCR Conditions for the Indicated Construct.

| DNA Construct | Primers | PCR conditions |
|---|---|---|
| IGFBP-2 | 5′-actg gaa ttc acc gcc agc atg ctg ccg aga-3′ | 98° C., 45 sec. |
| (1-163) | (SEQ ID NO: 9) | 69° C., 30 sec/72° C., 60 |
| | 5′-cagt gga tcc cta ctg cat ccg ctg ggt gt-3′ | sec |
| | (SEQ ID NO: 10) | (30 cycles) |
| | | 72° C., 7 min |
| IGFBP-2 | 5′-actg gaa ttc acc gcc agc atg aac cac gtg gac | 98° C., 45 sec. |
| (164-328) | agc acc at-3′ (SEQ ID NO: 11) | 69° C., 30 sec/72° C., 60 |
| | 5′-cagt gga tcc cta ctg cat ccg ctg ggt gt-3′ | sec |
| | (SEQ ID NO: 12) | (30 cycles) |
| | | 72° C., 7 min |
| IGFBP-2 | 5′-gaa ttc acc gcc agc atg ctg ccg aga-3′ | 98° C., 45 sec |
| (1-328) | (SEQ ID NO: 13) | 66° C., 30 sec/72° C., 60 |
| | 5′-gga tcc cta ctg cat ccg ctg ggt gt-3′ | sec |
| | (SEQ ID NO: 14) | (30 cycles) |
| | | 72° C., 7 min |

TABLE 3
Bacterial Sequence Homologies for IGFBP-2 C-Terminus

| | Number of positive | |
|---|---|---|
| | amino acids/total | |
| | amino acids | |
| Bacterial peptides with homology to IGFBP-2 (164-328) | examined | % positivity |
| WP_009027405.1\|ring-cleavage extradiol dioxygenase | 42/114 | 37 |
| [Bradyrhizobium sp. ORS 375] | | |
| NP_773913.1\|hypothetical peptide bll7273 | 41/111 | 37 |
| [Bradyrhizobium diazoefficiens USDA] | | |
| WP_010058321.1\|putative glyoxylasc peptide, partial | 33/95 | 35 |
| [Rhizobium etli] | | |
| WP_010066373.1\|putative glyoxylase, partial | 34/95 | 36 |
| [Rhizobium etli] | | |
| WP_008836585.1\|glyoxalase bleomycin resistance peptide | 39/95 | 41 |
| dioxygenase | | |
| [Mesorhizobium] | | |
| WP_008965179.1\|ring-cleavage extradiol dioxygenase | 38/106 | 36 |
| [Bradyrhizobium sp. STM 3809] | | |
| YP_005606980.1\|hypothetical peptide BJ6T_21130 | 41/111 | 37 |
| [Bradyrhizobium japonicum USDA | | |
| WP_003578050.1\|glyoxalase | 33/95 | 35 |
| [Rhizobium leguminosarum] | | |
| YP_005453204.1\|hypothetical peptide S23_59020 | 42/111 | 38 |
| [Bradyrhizobium sp.S23321] | | |
| WP_008528865.1\|glyoxylase, partial [Rhizobium sp. Pop5] | 33/95 | 35 |
| YP_007513616.1\|glyoxalase Bleomycin resistance peptide | 43/118 | 36 |
| dihydroxybiphenyl dioxygenase | | |
| WP_018453658.1\|ring-cleavage extradiol dioxygenase | 41/111 | 37 |
| [Mesorhizobium sp. WSM4349] | | |
| WP_007614565.1\|ring-cleavage extradiol dioxygenase | 41/111 | 37 |
| [Bradyrhizobium sp. WSM471] | | |
| WP_004674985.1\|glyoxalase [Rhizobium etli] | 33/95 | 35 |
| WP_007596546.1\|ring-cleavage extradiol dioxygenase | 41/111 | 37 |
| [Bradyrhizobium sp. WSM1253] | | |
| YP_008364002.1\|ring-cleaving dioxygenase peptide | 33/95 | 35 |
| [Rhizobium etli bv. Mimosae] | | |
| YP_468764.1\|ring-cleaving dioxygenase | 33/95 | 35 |
| [Rhizobium etli CFN 42] | | |
| WP_007760845.1\|glyoxalase | 36/101 | 36 |
| [Rhizobium sp. CF080] | | |
| WP_008563431.1\|hypothetical peptide | 40/111 | 36 |
| [Bradyrhizobium sp. CCGE-LA00]1 | | |
| YP_001205467.1\|glyoxalase Bleomycin resistance peptide | 30/82 | 37 |
| dihydroxybiphenyl dioxygenase | | |
| WP_018643731.1\|ring-cleavage extradiol dioxygenase | 43/115 | 37 |
| [Bradyrhizobium japonicum] | | |
| YP_916165.1\|glyoxalase bleomycin resistance peptide | 38/99 | 38 |
| dioxygenase | | |
| [Paracoccus] | | |
| WP_018901176.1\|glyoxalase | 34/95 | 36 |
| [Rhizobium sp. 2MFCol3.1] | | |
| WP_009798010.1\|hypothetical peptide | 37/97 | 38 |
| [Nitrobacter sp. Nb-311A] | | |
| WP_010017603.1\|putative glyoxylase peptide, partial | 33/95 | 35 |
| [Rhizobium etli] | | |
| YP_001819164.1\|glyoxalase bleomycin resistance peptide | 35/97 | 36 |
| dioxygenase | | |
| [Opitutus] | | |
| YP_007181098.1\|ring-cleavage extradiol dioxygenase | 36/94 | 38 |
| [Deinococcus peraridilitoris] | | |
| YP_004613302.1\|Glyoxalase bleomycin resistance peptide | 35/95 | 37 |
| dioxygenase | | |
| [Mesorhizobium] | | |
| WP_020039799.1\|hypothetical peptide | 33/93 | 35 |
| [Salipiger mucosus] | | |
| NP_101952.1\|hypothetical peptide mlr0078 | 36/95 | 38 |
| [Mesorhizobium loti | | |
| MAFF303099] | | |
| WP_006700058.1\|glyoxalase | 36/95 | 38 |
| [Rhizobium lupini] | | |
| WP_006150988.1\|glyoxalase | 33/81 | 41 |
| [Streptococcus infantis] | | |
| YP_007306117.1\|putative ring-cleavage extradiol dioxygenase | 34/93 | 37 |
| [Mesorhizobium australicum] | | |
| WP_006206040.1\|Glyoxalase bleomycin resistance peptide | 37/104 | 36 |
| dioxygenase | | |
| [Mesorhizobium] | | |
| YP_004143509.1\|glyoxalase bleomycin resistance peptide | 35/95 | 37 |
| dioxygenase | | |
| [Mesorhizobiuin] | | |
| WP_018859462.1\|glyoxalase | 33/95 | 35 |
| [Rhizobium sp. 42MFCr.1] | | |
| WP_018116556.1\|glyoxalase | 33/95 | 35 |
| [Rhizobium sp. JGI 0001005-H05] | | |
| WP_016466568.1\|hypothetical peptide | 34/81 | 42 |
| [Streptococcus sp. HPH0090] | | |
| WP_018239121.1\|glyoxalase | 30/85 | 35 |
| [Rhizobium sp. BR816] | | |
| WP_000262997.1\|glyoxalase | 34/81 | 42 |
| [Streptococcus infantis] | | |
| WP_006154005.1\|glyoxalase | 34/81 | 42 |
| [Streptococcus infantis] | | |
| WP_008139282.1\|ring-cleavage extradiol dioxygenase | 43/115 | 37 |
| [Bradyrhizobium sp. YR681] | | |
| YP_002886321.1\|Glyoxalase bleomycin resistance peptide | 34/94 | 36 |
| dioxygenase | | |
| [Exiguobacterium] | | |
| WP_003349449.1\|glyoxalase | 40/95 | 42 |
| [Bacillus methanolicus] | | |
| WP_003352106.1\|glyoxalase | 41/95 | 43 |
| [Bacillus methanolicus] | | |
| YP_007323883.1\|Glyoxalase bleomycin resistance peptide | 41/97 | 42 |
| dioxygenase | | |
| [Fibrella] | | |
| NP_244171.1\|hypothetical peptide BH3305 | 35/94 | 37 |
| [Bacillus halodurans C-125] | | |
| WP_007572105.1\|Glyoxalase family peptide | 32/89 | 36 |
| [Patulibacter sp. I11] | | |
| YP_001432381.1\|glyoxalase bleomycin resistance peptide | 33/83 | 40 |
| dioxygenase | | |
| [Roseiflexus] | | |
| WP_004254007.1\|glyoxalase | 33/81 | 41 |
| [Streptococcus mitis] | | |
| WP_010787986.1\|Catechol-2,3-dioxygenase subunit | 38/96 | 40 |
| [Bacillus | | |
| atrophaeus] | | |
| WP_021882458.1\|Catechol-2,3-dioxygenase | 39/95 | 41 |
| [Paenibacillus sp. P22] | | |
| WP_009732453.1\|hypothetical peptide | 36/92 | 39 |
| [Streptococcus sp. F0442] | | |
| WP_007791909.1\|glyoxalase | 33/95 | 35 |
| [Rhizobium sp. CF122] | | |
| YP_644103.1\|glyoxalase bleomycin resistance peptide | 37/97 | 38 |
| dioxygenase | | |
| [Rubrobacter] | | |
| YP_003972236.1\|catechol-2,3-dioxygenase subunit | 38/96 | 40 |
| [Bacillus atrophaeus1942] | | |
| WP_017436653.1\|glyoxalase | 36/95 | 38 |
| [Geobacillus caldoxylosilyticus] | | |
| YP_003010789.1\|glyoxalase bleomycin resistance peptide | 40/97 | 41 |
| dioxygenase | | |
| [Paenibacillus] | | |
| WP_007530969.1\|glyoxalase | 35/101 | 35 |
| [Rhizobium mesoamericanum] | | |
| WP_008478518.1\|glyoxalase | 32/85 | 38 |
| [Nitrolancetus hollandicus] | | |
| WP 003252641.1\|glyoxalase | 37/95 | 39 |
| [Geobacillus thermoglucosidasius] | | |
| YP_004589182.1\|Glyoxalase bleomycin resistance peptide | 37/95 | 39 |
| dioxygenase | | |
| [Geobacillus] | | |
| YP_003990495.1\|glyoxalase bleomycin resistance peptide | 37/95 | 39 |
| dioxygenase | | |
| [Geobacillus] | | |
| WP_002173545.1\|glyoxalase | 52/127 | 41 |
| [Bacillus cereus] | | |
| WP_016125785.1\|glyoxalase | 52/127 | 41 |
| [Bacillus cereus] | | |
| YP_005056176.1\|Glyoxalase bleomycin resistance peptide | 40/97 | 41 |
| dioxygenase | | |
| [Granulicella] | | |
| WP_006418304.1\|glyoxalase family peptide | 34/94 | 36 |
| [Eremococcus coleocola] | | |
| WP_006332912.1\|conserved hypothetical peptide | 33/95 | 35 |
| [Mesorhizobium sp.STM 4661] | | |
| WP_021151680.1\|Glyoxalase family peptide | 36/92 | 39 |
| [Streptococcus sp. IISISS3] | | |
| WP_008381503.1\|glyoxalase | 38/94 | 40 |
| [Enterococcus sp. C1] | | |
| WP_000009851.1\|glyoxalase | 51/127 | 40 |
| [Bacillus cereus] | | |
| YP_008690001.1\|glyoxalase | 38/92 | 41 |
| [Streptococcus sp. I-G2] | | |
| YP_008687719.1\|glyoxalase | 38/92 | 41 |
| [Streptococcus sp. I-P16] | | |
| WP_007608560.1\|Glyoxalase bleomycin resistance peptide | 34/95 | 36 |
| dioxygenase, partial | | |
| YP_008420225.1\|catechol-2,3-dioxygenase subunit | 38/96 | 40 |
| [Bacillus amyloliquefaciens] | | |
| YP_007496478.1\|catechol-2,3-dioxygenase subunit | 38/96 | 40 |
| [Bacillus amyloliquefaciens] | | |
| YP_007185508.1\|hypothetical peptide B938_04050 | 38/96 | 40 |
| [Bacillus amyloliquefaciens subsp.] | | |
| YP_008411815.1\|catechol-2,3-dioxygenase subunit | 38/96 | 40 |
| [Bacillus amyloliquefaciens] | | |
| YP_001420458.1\|hypothetical peptide RBAM_008430 | 38/96 | 40 |
| [Bacillus amyloliquefaciens] | | |
| YP_005129547.1\|hypothetical peptide BACAU_0818 | 38/96 | 40 |
| [Bacillus amyloliquefaciens subsp.] | | |
| WP_008534992.1\|glyoxalase | 30/81 | 37 |
| [Streptococcus sp. C150] | | |
| YP_005540439.1\|hypothetical peptide BAMTA208_03815 | 37/96 | 39 |
| [Bacillus amyloliquefaciens] | | |
| WP_017763607.1\|glyoxalase [Bacillus thuringiensis] | 52/127 | 41 |
| NP_834066.1\|glyoxalase family peptide | 52/127 | 41 |
| [Bacillus cereus ATCC 14579] | | |
| WP_000009842.1\|glyoxalase | 52/127 | 41 |
| [Bacillus cereus] | | |
| WP_000009838.1\|glyoxalase | 52/127 | 41 |
| [Bacillus cereus] | | |
| YP_006606889.1\|glyoxalase | 52/127 | 41 |
| [Bacillus thuringiensis HD-771] | | |
| YP_003666551.1\|glyoxalase | 52/127 | 41 |
| [Bacillus thuringiensis BMB171] | | |
| YP_008625416.1\|hypothetical peptide BAPNAU_0774 | 37/96 | 39 |
| [Bacillus amyloliquefaciens] | | |
| YP_005420099.1\|hypothetical peptide BANAU_0761 | 37/96 | 39 |
| [Bacillus amyloliquefaciens subsp] | | |
| YP_005574328.1\|glyoxalase family peptide | 52/127 | 41 |
| [Bacillus thuringiensis serovar chinensis] | | |
| YP_001376297.1\|glyoxalase bleomycin resistance peptide | 34/95 | 36 |
| dioxygenase | | |
| [Bacillus] | | |
| WP_017657192.1\|hypothetical peptide | 52/127 | 41 |
| [Bacillus sp. WBUNB009] | | |
| WP_000009846.1\|glyoxalase | 52/127 | 41 |
| [Bacillus cereus] | | |
| WP 000009847.1\|glyoxalase | 52/127 | 41 |
| [Bacillus cereus] | | |
| WP_000009848.1\|glyoxalase | 52/127 | 41 |
| [Bacillus cereus] | | |
| YP_002369169.1\|glyoxalase | 52/127 | 41 |
| [Bacillus cereus B4264] | | |
| WP_021147245.1\|Glyoxalase family peptide | 32/81 | 40 |
| [Streptococcus sp. HSISS4] | | |
| WP_016080228.1\|glyoxalase | 51/127 | 40 |
| [Bacillus cereus] | | |
| WP_007085978.1\|glyoxalase family peptide | 34/92 | 37 |
| [Bacillus bataviensis] | | |
| YP_006070527.1\|ring-cleavage extradiol dioxygenase | 31/81 | 38 |
| [Streptococcus salivarius | | |
| WP_018394429.1\|hypothetical peptide | 37/95 | 39 |
| [Bacillus sp. 37MA] | | |
| WP_000009837.1\|glyoxalase | 52/127 | 41 |
| [Bacillus thuringiensis] | | |
| WP 016086201.1\|glyoxalase | 52/127 | 41 |
| [Bacillus cereus] | | |
| NP_980734.1\|glyoxalase family peptide | 52/127 | 41 |
| [Bacillus cereus ATCC 10987] | | |
| YP_006596780.1\|glyoxalase | 52/127 | 41 |
| [Bacillus cereus FRI-35] | | |
| WP_000009828.1\|glyoxalase | 52/127 | 41 |
| [Bacillus cereus] | | |
| WP_000009827.1\|glyoxalase | 52/127 | 41 |
| [Bacillus cereus] | | |
| WP_000009824.1\|glyoxalase | 52/127 | 41 |
| [Bacillus cereus] | | |
| WP_000009823.1\|glyoxalase | 52/127 | 41 |
| [Bacillus cereus] | | |
| WP_000009829.1\|glyoxalase | 52/127 | 41 |
| [Bacillus cereus group] | | |
| YP_003700984.1\|glyoxalase bleomycin resistance peptide | 36/93 | 39 |
| dioxygenase | | |
| [Bacillus] | | |
| WP_018387644.1\|hypothetical peptide | 33/94 | 35 |
| [Xanthobacteraceae] | | |
| WP_000009812.1\|glyoxalase | 52/127 | 41 |
| [Bacillus cereus] | | |
| WP_003094776.1\|glyoxalase | 30/81 | 37 |
| [Streptococcus vestibularis] | | |
| WP_016718514.1\|glyoxalase | 52/127 | 41 |
| [Bacillus cereus] | | |
| YP_005555895.1\|YfiE | 38/96 | 40 |
| [Bacillus subtilis subsp. subtilis str. RO-NN-1] | | |
| WP 021143928.1\|Glyoxalase family peptide | 32/81 | 40 |
| [Streptococcus sp. HSISS1] | | |
| WP_002891269.1\|glyoxalase | 32/81 | 40 |
| [Streptococcus salivarius] | | |
| YP_030504.1\|glyoxase | 50/127 | 39 |
| YP_007423766.1\|Glyoxalase | 52/127 | 41 |
| [Bacillus thuringiensis serovar kurstaki str.HD73] | | |
| WP_000009844.1\|glyoxalase | 52/127 | 41 |
| [Bacillus cereus group] | | |
| WP_000009835.1\|glyoxalase | 52/127 | 41 |
| [Bacillus cereus] | | |
| WP_000009832.1\|glyoxalase | 52/127 | 41 |
| [Bacillus cereus] | | |
| WP_000009836.1\|glyoxalase | 52/127 | 41 |
| [Bacillus cereus] | | |
| WP_000009833.1\|glyoxalase | 52/127 | 41 |
| [Bacillus cereus] | | |
| WP_000009834.1\|glyoxalase | 52/127 | 41 |
| [Bacillus cereus] | | |
| WP_003302787.1\|glyoxalase | 52/127 | 41 |
| [Bacillus thuringiensis] | | |
| YP_006230710.1\|catechol-2,3-dioxygenase subunit | 37/96 | 39 |
| [Bacillus sp. JS] | | |
| WP_010284708.1\|glyoxalase | 37/95 | 39 |
| [Bacillus sp. 10403023] | | |
| WP_016621174.1\|glyoxalase family peptide | 38/94 | 40 |
| [Enterococcus faecalis] | | |
| WP_005236471.1\|glyoxalase | 38/94 | 40 |
| [Enterococcus casseliflavus] | | |
| WP_000009850.1\|glyoxalase | 51/127 | 40 |
| [Bacillus cereus] | | |
| YP_007753120.1\|hypothetical peptide ECBG_02114 | 38/94 | 40 |
| [Enterococcus casseliflavus EC20] | | |
| WP_005229961.1\|glyoxalase | 38/94 | 40 |
| [Enterococcus casseliflavus] | | |
| WP_016610999.1\|glyoxalase family peptide | 38/94 | 40 |
| [Enterococcus casseliflavus] | | |
| WP_010749563.1\|hypothetical peptide | 38/94 | 40 |
| [Enterococcus casseliflavus] | | |
| YP_005567940.1\|glyoxalase family peptide | 52/127 | 41 |
| [Bacillus thuringiensis serovar finitimus] | | |
| YP_007426021.1\|hypothetical peptide C663_0846 | 38/96 | 40 |
| [Bacillus subtilis XF-1] | | |
| YP_007210464.1\|hypothetical peptide A7A1_0594 | 38/96 | 40 |
| [Bacillus subtilis subsp. Subtilis] | | |
| YP_005560045.1\|hypothetical peptide BSNT_01369 | 38/96 | 40 |
| [Bacillus subtilis subsp. Natto] | | |
| NP_388705.2\|catechol-2,3-dioxygenase subunit | 38/96 | 40 |
| [Bacillus subtilis subsp. subtilis] | | |
| YP_006396067.1\|catechol-2,3-dioxygenase | 33/95 | 35 |
| [Sinorhizobium fredii USDA 257] | | |
| WP_000009821.1\|glyoxalase | 51/127 | 40 |
| [Bacillus cereus] | | |
| WP_018672727.1\|glyoxalase | 34/95 | 36 |
| [Brevibacillus laterosporus] | | |
| WP_000009856.1\|glyoxalase | 52/127 | 41 |
| [Bacillus cereus] | | |
| YP_003794080.1\|glyoxalase | 49/127 | 39 |
| [Bacillus cereus biovar anthracis str. CI] | | |
| WP_016181685.1\|hypothetical peptide | 36/94 | 38 |
| [Enterococcus avium] | | |
| WP_000009830.1\|glyoxalase | 52/127 | 41 |
| [Bacillus cereus] | | |
| WP 010497223.1\|glyoxalase | 34/94 | 36 |
| [Lactobacillus acidipiscis] | | |
| YP_008631724.1\|conserved hypothetical peptide | 33/93 | 35 |
| WP_006699989.1\|ring-cleavage extradiol dioxygenase | 33/93 | 35 |
| [Rhizobium lupini] | | |
| WP_016765267.1\|glyoxalase | 38/95 | 40 |
| [Bacillus megaterium] | | |
| YP_003564423.1\|glyoxalase family peptide | 38/95 | 40 |
| [Bacillus megaterium QMB1551] | | |
| YP_003694888.1\|glyoxalase bleomycin resistance peptide | 37/94 | 39 |
| dioxygenase | | |
| [Starkeya] | | |
| WP_017868743.1\|glyoxalase | 33/94 | 35 |
| [Lactobacillus pobuzihii] | | |
| WP_019156447.1\|hypothetical peptide | 37/94 | 39 |
| [Bacillus massiliosenegalensis] | | |

TABLE 4
Human Peptide Sequence Homologies for IGFBP-2 N-Terminus.

| | Number of | |
|---|---|---|
| | positive amino | |
| | acids/total | |
| | amino acids | % |
| Protein with homology to IGFBP-2 (1-163) | examined | positivity |
| Insulin-like growth factor-binding peptide 3 | 55/106 | 52 |
| Insulin-like growth factor-binding peptide 5 | 51/97 | 53 |
| Insulin-like growth factor-binding peptide 4 | 61/109 | 56 |
| Insulin-like growth factor-binding peptide 1 | 58/116 | 50 |
| Insulin-like growth factor-binding peptide 6 | 41/97 | 42 |
| Protein CYR61 | 31/81 | 38 |

TABLE 5
Human Peptide sequence Homologies for IGFBP-2 C-Terminus.

| | Number of | |
|---|---|---|
| | positive amino | |
| | acids/total | |
| Protein with | amino acids | % |
| homology to IGFBP-2 (164-328) | examined | positivity |
| Insulin-like growth factor-binding peptide 1 | 58/93 | 62 |
| Insulin-like growth factor-binding peptide 4 | 59/100 | 59 |
| Insulin-like growth factor-binding peptide 5 | 59/124 | 48 |
| Nidogen-1 | 35/90 | 39 |
| Insulin-like growth factor-binding peptide 3 | 42/83 | 51 |
| Nidogen-2 | 33/84 | 39 |
| HLA class II histocompatibility antigen | 39/96 | 41 |
| gamma chain | | |
| Testican-3 | 41/100 | 41 |
| Thyroglobulin | 41/107 | 38 |
| Testican-1 | 40/99 | 40 |
| Insulin-like growth factor-binding peptide 6 | 44/82 | 54 |
| Testican-2 | 38/82 | 46 |
| SPARC-related modular calcium-binding | 31/82 | 38 |
| peptide 2 | | |
| SPARC-related modular calcium-binding | 37/100 | 37 |
| peptide 1 | | |

### Example 3

### IGFBP-2 Epitope-Specific Th2 Demonstrate a Higher Functional Avidity and Homology to a Greater Number of Bacterial and Self-Peptides than IGFBP-2 Epitope-Specific Th1

This example shows that the IGFBP-2 epitope-specific Th2 demonstrate a higher functional avidity and homology to a greater number of bacterial and self-peptides than IGFBP-2 epitope-specific Th1. Titration studies documented that the peptides that induced IL-10 secretion were recognized by T-cells with a higher functional avidity (mean EC₅₀ concentration: 0.12±0.02 μg/ml) (see FIG. 11) than those peptides that induced an IFNγ response (mean EC₅₀ concentration 2.1±0.43 μg/ml; p=0.014). FIG. 11 shows that an N-terminus, but not C-terminus, IGFBP-2 vaccine both stimulates Type I immunity and inhibits tumor growth. (A) IFNγ ELISPOT in splenocytes from mice immunized with the indicated vaccine. The data are presented as corrected spots per well (CSPW). The horizontal bar indicates the mean CSPW±SEM. n=10 mice/group; *p<0.01. (B) Mean tumor volume (mm³±SEM) from mice injected with pUMVC3 alone (), pUMVC3-hIGFBP2 (1-328) (▪), pUMVC3-hIGFBP2 (164-328) (▴) or pUMVC3-hIGFBP2 (1-163) (∘). n=5 mice/group; **p<0.001.

The N- and C-terminus differed in the amount of sequence homologies shared with foreign antigens. 157 bacterial species that demonstrated 35% shared amino acid positivity over 80 or more amino acids (range 35-43%) for the human IGFBP-2 C-terminus were identified (see Table 3). In contrast, the N-terminus demonstrated no sequence homology with bacterial peptides, a difference of over 100-fold. There was no difference in the number of viral homologies between the two termini (N-term, 0 and C-term, 0).

The IGFBP-2 N-terminus shared significant homology with other IGFBP peptides, and only one additional self-peptide, CYR61 (see Table 4). The C-terminus also demonstrated significant homology with other IGFBP peptides but also to nine additional self-peptides including thyroglobulin, nidogens, and testicans (see Table 5). Only 16% of all homologous sequences for the N-terminus were non-IGFBP related while 64% of homologous sequences for the C-terminus were self-peptides other than IGFBP family members.

For example, FIG. 12 shows IGFBP-2 vaccine-induced Th2 abrogates the anti-tumor effect of IGFBP-2-specific Th1. Type II cytokines IL-4 and IL-10 (A) and Type I cytokines TNFα and IFNγ (B) secretion from T-cell lines expanded with peptides in IGFBP2 (1-163) or IGFBP2 (164-328) (mean ng/mI±SD); **p<0.001, *p<0.01 and #p<0.05. (C) Mean tumor volume (mm³±SEM) from mice infused with CD3+ T-cells expanded from mice vaccinated with pUMVC3-hIGFBP2 (1-163) (∘), pUMVC3-hIGFBP2 (164-328) (▴) or naïve T-cells (). n=4 mice/group; *p<0.01. (D) Mean tumor volume (mm³±SEM) from mice injected with pUMVC3 alone (), pUMVC3-hIGFBP2 (164-328) (▴), pUMVC3-hIGFBP2 (1-163) (∘) or pUMVC3-hIGFBP2 (1-163)+pUMVC3-hIGFBP2 (164-328) (▾). n=5 mice/group; *p<0.01.

### Example 4

### Screening Breast Cancer Patient Lymphocytes for Reactivity to IFNγ and IL-10 ELISPOT

This example demonstrates screening of breast cancer patient lymphocytes with IFNγ and IL-10 ELISPOT for epitopes in the N-terminus of IGFBP-2 that elicited predominantly Th1 while the C-terminus stimulated Th2 and mixed Th1/Th2 responses. Epitope-specific Th2 demonstrated a higher functional avidity for antigen than epitopes which induced IFNγ (p=0.014).

TgMMTV-neu mice were immunized with DNA constructs encoding IGFBP-2 N- and C-termini. T-cell lines expanded from the C-terminus vaccinated animals secreted significantly more Type II cytokines than those vaccinated with the N-terminus and could not control tumor growth when infused into tumor-bearing animals. In contrast, N-terminus epitope-specific T-cells secreted Th1 cytokines and significantly inhibited tumor growth, as compared with naïve T-cells, when adoptively transferred (p=0.005) (see Tables 2-5).

To determine whether removal of Th2 inducing epitopes had any effect on the vaccinated anti-tumor response, mice were immunized with the N-terminus, C-terminus and a mix of equivalent concentrations of both vaccines. The N-terminus vaccine significantly inhibited tumor growth (p<0.001) as compared to the C-terminus vaccine which had no anti-tumor effect. Mixing the C-terminus with the N-terminus vaccine abrogated the anti-tumor response of the N-terminus vaccine alone (see Tables 2-5).

### Example 5

### Epitopes Derived from Stem Cell/EMT Antigens Preferentially Elicit T-Cells that Secrete IFNγ or IL-10 in Breast Cancer Patients and Selection of Peptides as Candidate Vaccine Epitopes that have Low Immune Suppressive Potential

This example demonstrates an evaluation of epitopes derived from stem cell/EMT antigens preferentially elicit T-cells that secrete IFNγ or IL-10 in breast cancer patients. Peptides as candidate vaccine epitopes that have low immune suppressive potential may be chosen. Peptides will be screened for immunogenicity using an IFNγ ELISPOT assay as previously described. A positive response will be defined as a precursor frequency that is significantly (p<0.05) greater than the mean of no-antigen wells. Leukapheresis products from 40 breast cancer patients and 40 controls have been archived perform these assays.

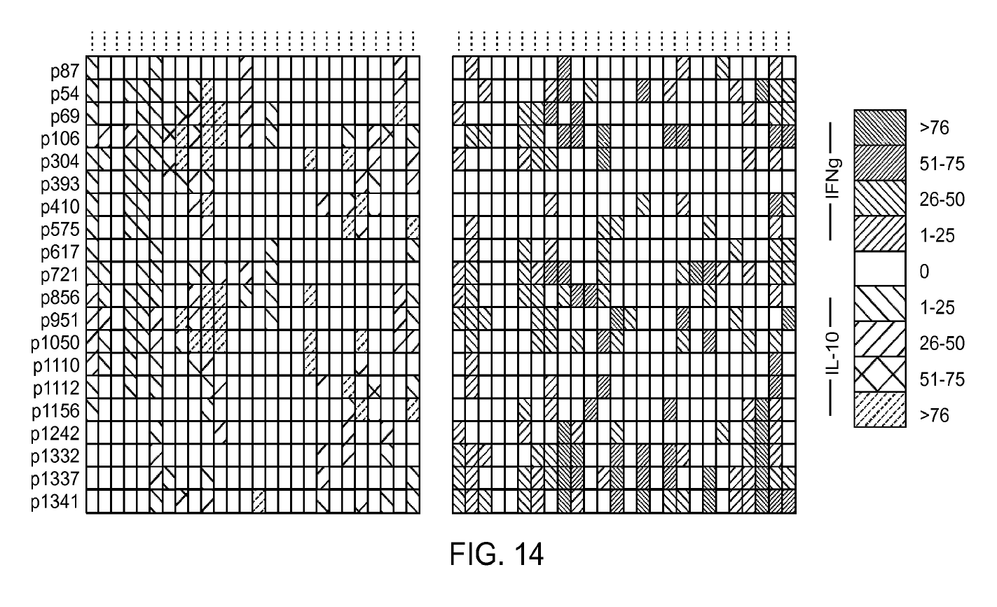

Identification of class II epitopes that might preferentially enhance the growth of Th2 or self-regulatory T-cells would allow such peptides to be excluded from any vaccine formulation. Epitope specific IL-10 secretion to exclude suppressive peptides will be evaluated via IL-10 ELISPOT using methods that have been previously reported as well as shown in FIG. 14.

Short term peptides will be used to identify whether peptide specific Th1 cells respond to peptide presented on endogenous APC (e.g., native epitopes). Short term peptide specific T-cell lines will be generated using methods described herein and demonstrate that the candidate Th1 peptide generated T-cells respond to native peptide and not an irrelevant peptide (such as myoglobin) via IFNγ ELISPOT. Commercially available recombinant peptides will be used as the source of antigen. Response to peptide and peptide antigens is considered to be positive as described above. The epitopes will be validated as Class II binding by conducting class II MHC blocking assays on the generated T-cell lines. Those peptides that elicit both peptide and peptide specific reactivity will be further considered as part of a stem cell/EMT targeted vaccine.

### Example 6

### Screening Breast Cancer Patient Lymphocytes for Reactivity to IFNγ and IL-10 ELISPOT

This example describes Phase I clinical vaccine trials that have been initiated to evaluate the potential for immediate toxicity due to intradermal (i.d.) vaccination. More than 200 subjects have received GM-CSF (e.g., 100-150 μg) admixed with HER2 peptide/peptide/or DNA-based vaccines administered i.d. monthly for 3-6 months. The cumulative toxicity data from patients enrolled on those trials revealed no grade 3 or 4 toxicity.

Patients have been evaluated for the potential of toxicity due to immunologic consequences of vaccination. Targeting HER2 has not resulted in untoward toxicity after vaccination. In order to assess potential toxicity, subjects will continue to be evaluated at each visit based on the modified NCI toxicity criteria as well as a complete physical examination. In addition, serum chemistries, including renal function tests, uric acid, blood counts, serum glucose, and liver function tests will be evaluated. The development of connective tissue disorders and laboratory autoantibody responses will also be clinically assessed as a potential immunologic toxicity associated with the use of DNA vaccination would be the development of anti-DNA antibodies. Therefore, anti-ANA, anti-C3, anti-thyroid and ds-DNA antibodies will be assessed prior to and at the end of the vaccination regimen, and at 12 months of follow-up.

The sample size of 22 subjects was chosen such that if no toxicities occur, the probability of such an occurrence is at least 90% if the true toxicity rate, e.g. any Grade 3 or 4 toxicity, is 10% or less. Such an occurrence will be taken as preliminary evidence that the true toxicity rate is less than 10%. The study will continue and be deemed sufficiently safe as long as the observed toxicity rate is consistent with a true grade 3 rate of 15% or less and a true grade 4 rate of 5% or less. Towards this end, stopping rules will be in place so that if there exists sufficient evidence to suggest true toxicity rates in excess of these thresholds, the study will be stopped. Sufficient evidence will be taken to be a lower one-sided confidence limit in excess of the appropriate threshold. For grade 3, such a limit will be reached if this level of toxicity occurred in 2 of the first 3 or fewer, 3 of the first 7 or fewer, 4 of the first 12 or fewer, 5 of the first 17 or fewer, or 6 of the first 22 or fewer enrolled patients. For grade 4, any of the following would lead to stopping: 2 of the first 10 or fewer, 3 of the first 22 or fewer enrolled patients experience grade 4 toxicity. If the true probability of grade 3 toxicity is 10% or 30%, then the probability of stopping the study is approximately 0.06 and 0.76, respectively. If the true probability of grade 4 toxicity is 3% or 23%, then the probability of stopping is roughly 0.05 and 0.93, respectively (probabilities estimated from 5,000 simulations).

The immunogenicity of a stem cell/EMT multi-antigen polyepitope vaccine in patients with triple negative breast cancer may be determined. Using 22 patients, the study can be 80% confident that the estimated immunologic response rate is within at least 0.14 of the true response rate. Spearman's correlation coefficient will be used to estimate the correlation between two continuous measures.

The generation of antigen specific T-cell immunity elicited after immunization may be determined by using a vaccine strategy that focuses on the generation of Th1 immunity. For this reason, our primary immunologic analysis will be focused on defining the magnitude of the Th1 antigen specific immune response using IFNγ ELISPOT. Assay validation was established in preliminary studies using the HLA-A2 flu peptide and tetanus peptide over a PBMC range of 1.0-3.5×10⁵ cells and also with the use of IFNγ-coated polystyrene beads in 20 donor PBMC. These studies demonstrated that the assay is linear and precise between 2.0 and 3.5×10⁵ PBMC/well, has a detection limit of 1:60,000, and has a detection efficiency of 93%. Pre-vaccine and post-vaccine samples will be analyzed simultaneously to correct for variability.

The antigens to be evaluated are: 1 ug/ml peptide antigens (recombinant peptides are available on all of the proposed candidate antigens, human myoglobin (negative control)) or 1 μg/ml CMV lysate and 0.5 U/ml tt (positive controls) and peptide antigens encompassed within the vaccine at 10 μg/ml. A patient is considered to be successfully immunized if the patient develops peptide specific precursor frequencies more robust than 1:20,000 PBMC to the majority of the immunizing antigens. If patients have pre-existent immunity to any of the antigens, then their responses must augment over 2 times baseline response to be considered “immunized”.

The Antigen Specific Th Phenotype Elicited after Immunization May be Determined.

The vaccination strategy may be to elicit highly skewed Th1 antigen specific T-cells to multiple stem cell/EMT antigens. The assessment of cytokine secretion by antigen specific T-cells phenotypes the vaccinated response. Supernatants may be removed 72 hours after antigen stimulation in the ELISPOT assay. The supernatants may be evaluated for a panel of cytokines by multiplex analysis, for example, Th1 (IFNγ, IL-2, TNF-α, IL-1b, GM-CSF) and Th17 (IL-17), and Th2 (IL-6, IL-4, IL-10, IL-13) cytokines. The cytokine panel may be supplemented with TGF-β in an ELISA format.

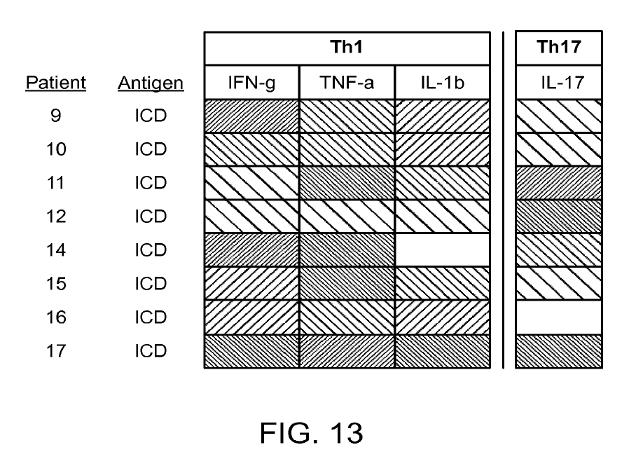

Supernatants from ELISPOT assays were collected during the conduct of a Phase II study of a HER2 peptide vaccine. FIG. 13 shows exemplary data on 8 advanced stage HER2+ breast cancer patients receiving vaccinations. Values collected via cytokine multiplexing are color coded as to the magnitude of antigen specific cytokine increase (red) or decrease (blue) with vaccination (displayed as a cytokine “heat map”). The intensity of the colors symbolizes lowest (pale) to highest (vivid) quartile of response. The data suggest specific patterns of Th response to the HER2 ICD peptide (immunizing antigen); Th1/17, Th2, and “mixed”. Patient 12 and 17 increased HER2 specific Type 1 cytokine and IL-17 secretion with vaccination. This type of response is similar to what would be expected after immunization with a vaccine designed to elicit Th1 immunity. Patient 16 decreased both HER2 specific Th1 and Th17 cytokine production. This phenotype may limit the development or retention of tumor antigen specific immunity.

Statistical Analysis.

The unpaired, two-tailed Student's t-test, Fischer's exact test or X² test was used to evaluate differences between groups. The half maximal concentration (EC50) of peptide was calculated as log(agonist) vs. response and reported as mean±SEM for five donors with IFNγ and four donors for IL-10 responses (one IL-10 donor demonstrated no dose tritration at the concentrations evaluated). p<0.05 was considered significant. All statistical analyses were performed using GraphPad Prism 5.04 (GraphPad Software).

Based on the observations that approximately 25% of patients in the study described in FIG. 13 had a Type I “good” response, a response rate may be set at 25% as the benchmark by which this treatment will be evaluated for success. If the true response rate is 60%, 22 patients provide 97% power to observe a statistically significantly improved response rate compared to the fixed rate of 25% (one-sided level of significance of 0.05). If the true response rate is 50%, the power is 82%.

### Example 7

### Evaluation of Stem Cell/EMT Antigen-Derived Epitopes Preferentially Elicit T-Cells that Secrete IFNγ or IL-10 and Selection of Peptides as Candidate Vaccine Epitopes with Low Immune Suppressive PotentialSamples Screened by IL-10 ELISPOT

Identification of Peptides that Induced Antigen Specific IFNγ Secreting T-Cells Compared to IL-10 Secreting T-Cells.

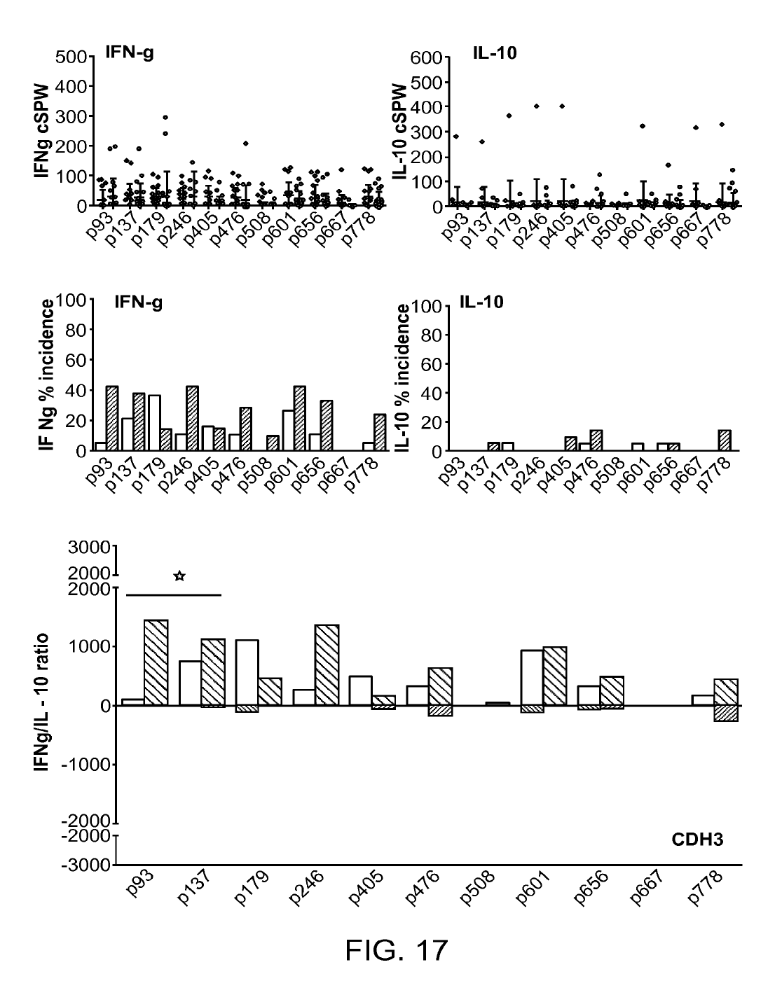

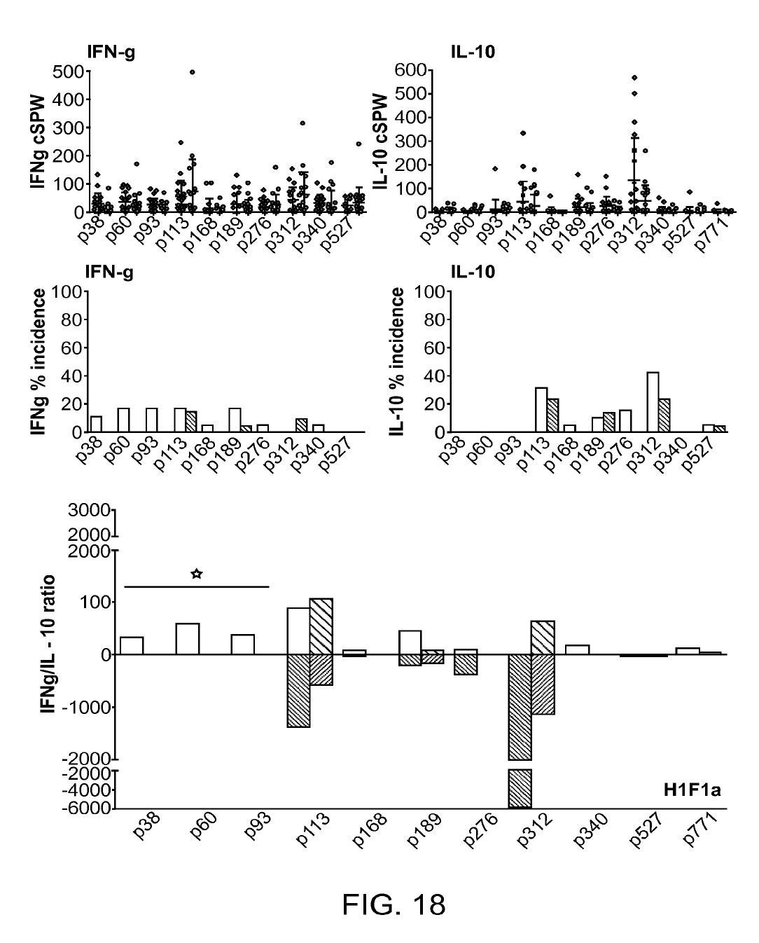

A matrix scoring system that prioritized antigens for in vivo evaluation of extended epitopes that demonstrated IFNγ specific activity in the absence of IL-10 activity across the populations was used. Extended epitopes were superior to shorter class II epitopes in that the longer epitopes elicited a diverse immune response consisting of both T and B-cells and anti-tumor responses were dependent on both CD4 and CD8 T-cells. Regions in a candidate antigen that contained multiple epitopes that preferentially induced a greater magnitude and incidence of IFNγ responses and little or no IL-10 inducing activity were identified. A ratio (IFNγ/IL-10 activity ratio) of the incidence×magnitude of antigen specific IFNγ induction/the incidence×magnitude of antigen specific IL-10 induction was evaluated (see FIGS. 17 and 18).

For example, FIG. 18, shows the lower magnitude and incidence IFNγ predominance. IFNγ/IL-10 activity ratios for selected antigens. IFNγ/IL-10 ratio, defined as the mean cSPW×incidence per peptide, shown by donor type. IFNγ cSPW×incidence shown on the positive y-axis for volunteer donors, shown in white bars, and cancer donors, shown in white bars with black pattern. IL-10 cSPW×incidence shown on the negative y-axis for volunteer donors, shown in black bars, and cancer donors, shown in black bars with white pattern. (A) CDH3, (B) HIF1α, (C) survivin, and (D) FOXQ1.

The evaluated antigens were categorized into 4 major groups based on the IFNγ/IL-10 activity ratio. The first group, exemplified by CDH3 (FIG. 17) displayed a high incidence/magnitude IFNγ response with very little IL-10 activity. This pattern indicated a top tier antigen and included CDH3, SOX2, MDM2, and Yb-1. The second tier antigens demonstrated a similar predominant IFNγ response with minimal to no IL-10 induction in regions of selected extended epitopes, however the magnitude of the immune response was greater than a log lower than the top tier candidates. FIG. 18, HIF1α, exemplifies this category which also includes CD105, CDC25B, and SATB1. Vaccine candidates were derived from these categories (see FIGS. 17 and 18).

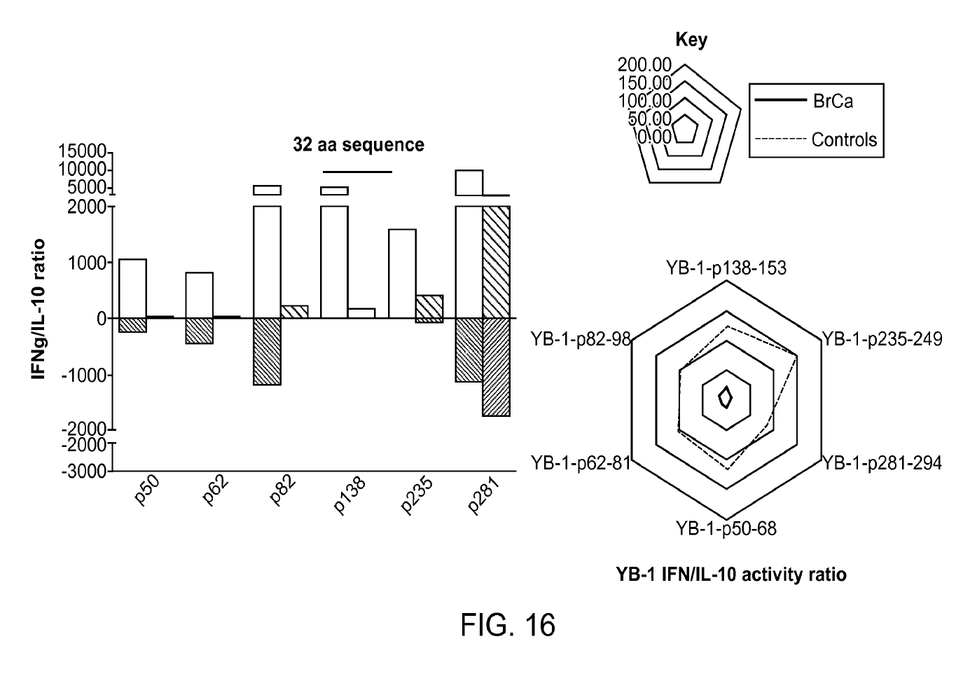

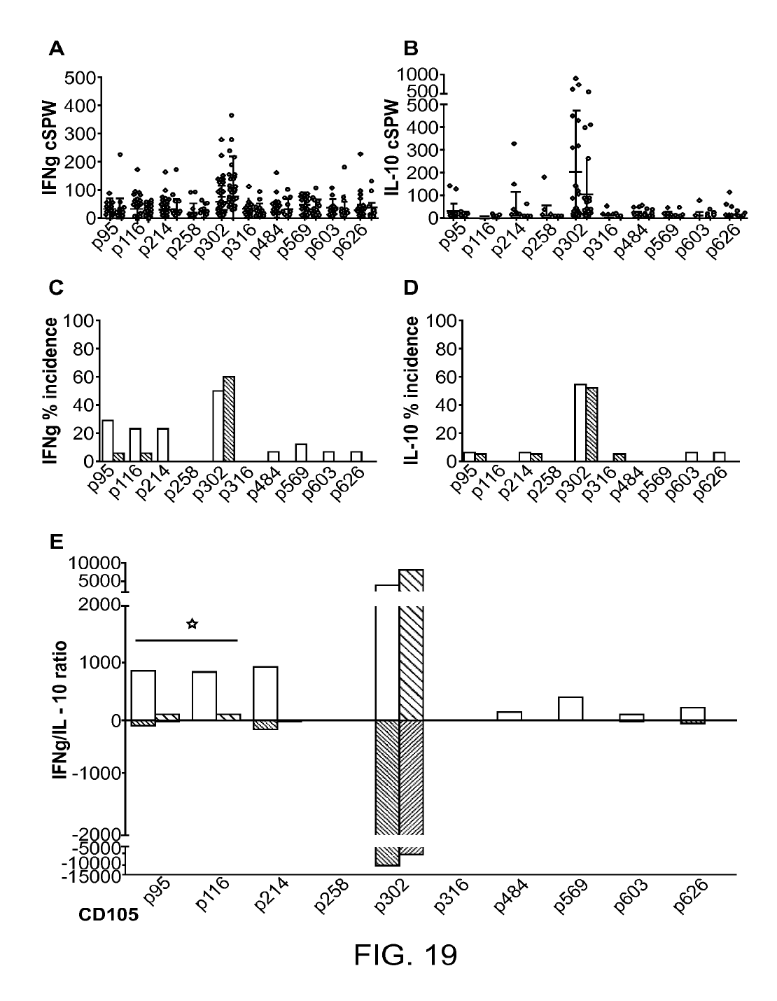

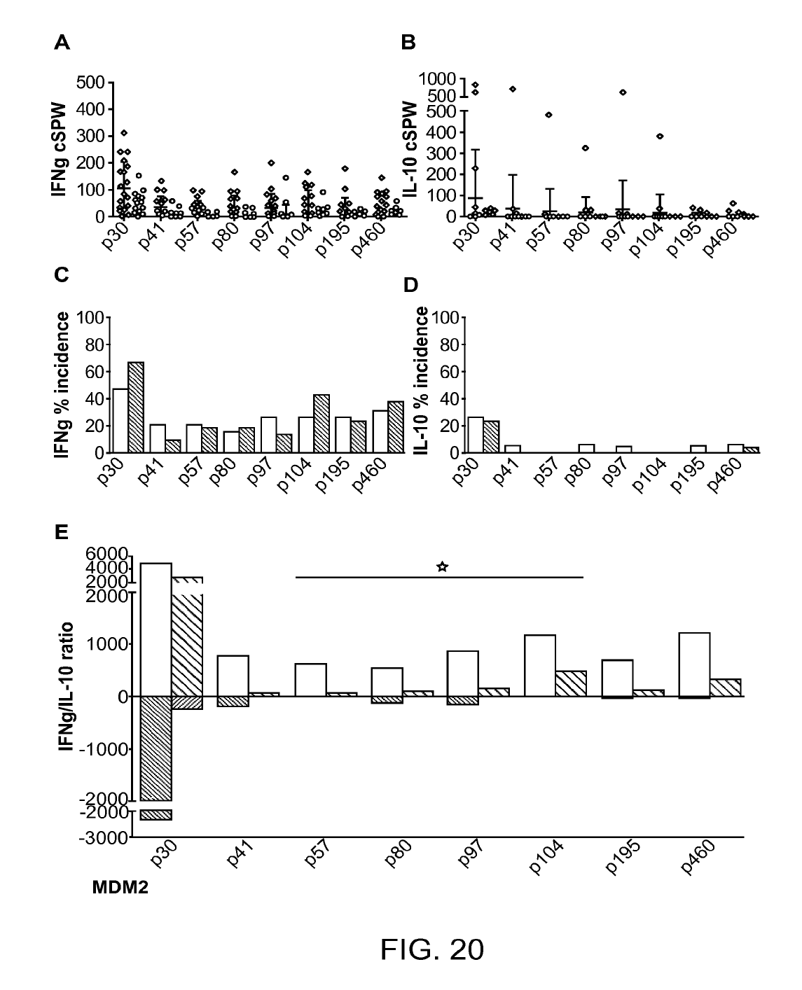

The other two groups had characteristics which were much less desirable for a vaccine immunogen. Although there are epitopes that stimulate a high magnitude and incidence IFNγ response, these sequences equally induce high magnitude IL-10 immunity at an equal incidence in tested individuals. Antigens such as c-met, IGF-1R, PRL3, and SIX1 are grouped into this category. Finally, some candidate antigens were not immunogenic as demonstrated by both a low incidence as well as low magnitude of any immune response, as shown in FIG. 19 for FOXQ1. ID1 and SNAIL also were associated with very low incidence and magnitude of immune response. Immunogenic and inducing high magnitude and incidence IL-10 responses or weakly immunogenic, these latter two categories of antigens will be excluded from further consideration for the final vaccine formulation. In FIG. 16, a list of extended epitopes based on IFNγ/IL-10 activity ratio is shown. See also FIGS. 17-20.

### Example 8

### Construction of a Multiantigen Th1 Polyepitope Plasmid Based Vaccine Targeting Stem Cell/EMT Antigens and Determination of Safety and Immunogenicity

Determination of Immunogenicity and Effectiveness of Plasmid Based Vaccine Constructs Containing Either or Both Short Th Epitopes or Extended Th Epitopes Using the TgMMTVneu Mouse Model with the IGF-1R Antigen.

In order to directly compare the ability of the short and extended epitope plasmid vaccines to control tumor growth, a syngeneic tumor implant model was employed. Mice (TgMMTVneu) were separated into 4 vaccination groups (pIGF-IRexep, pIGF-IRshep, vector, and IGF-IR peptides) and implanted with syngeneic breast cancer cells (MMC) 7 days after the 3rd vaccination. Dosages were as stated above. The ability of MMC cells to form a tumor, and the tumor growth rate was measured. The IGF-IR peptide vaccine, the short epitope plasmid vaccine, and the extended epitope plasmid vaccine all significantly controlled tumor growth compared to the group that was vaccinated with vector alone (p<0.0001, from 14-31 days). The mice vaccinated with pIGF-IRexep had the slowest growing tumors, but they were not significantly different from tumor growth in animals vaccinated with pIGF-IRshep, p>0.05.

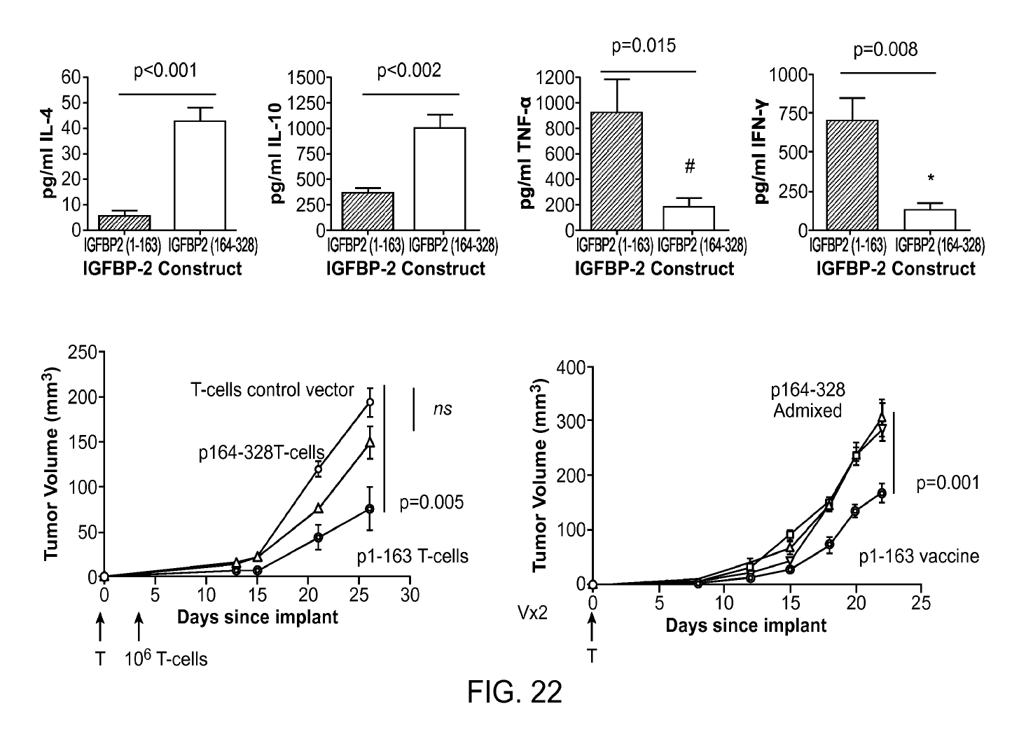

For example, FIG. 22 Th2 immune responses abrogate the anti-tumor efficacy of Th1 immune responses.

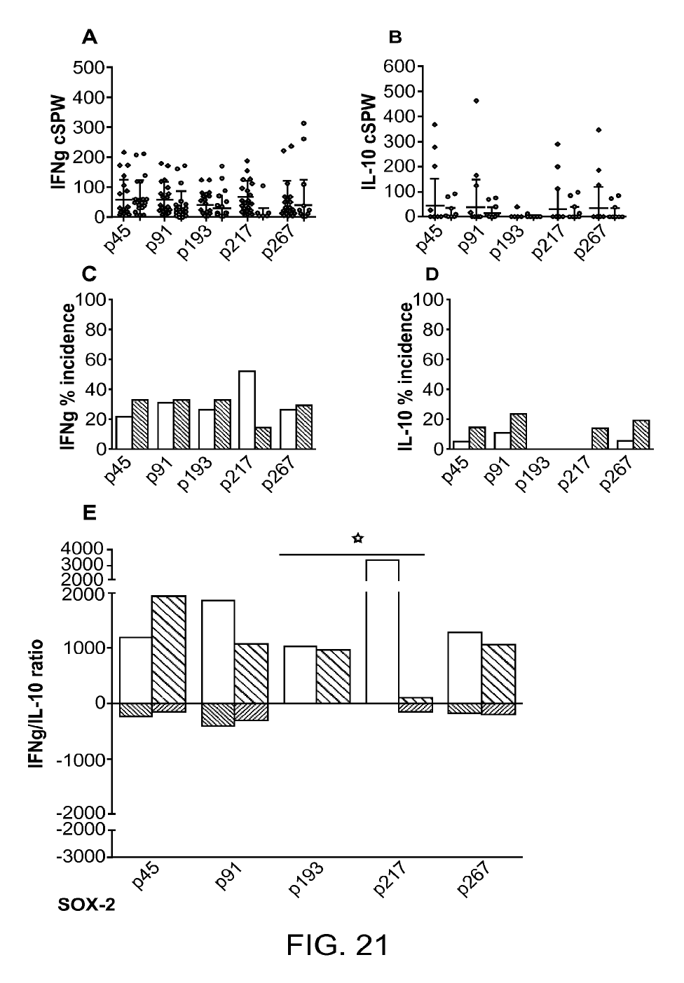

For example, FIG. 21 HIF1α peptide and plasmid vaccine immunogenicity and efficacy were determined in mice. (A) DTH responses were measured by change in ear thickness (mm) 24 hours after application of HIF1α peptide mix in 50% DMSO. Plotted are responses of individual FVB/NJ mice from the different vaccination cohorts: Controls (both adjuvant only and vector groups, see Methods), HIF1α Peps (peptide vaccine), and pHIF1α (plasmid vaccine). Dotted line represents 0.0 mm change in ear thickness from baseline. *** p<0.001 vs. controls. (B) DTH responses measured 24 hours after application of HIF1α peptide mix in 50% DMSO. Plotted are responses of individual MMTV-C3(1)-Tag transgenic mice from the different vaccination cohorts as listed above. *** p<0.001 vs. controls. (C) Efficacy of vaccines to control M6 tumor growth was assessed by measuring tumor volume (mm3) over time post-implant (days) in MMTV-C3(1)-Tag transgenic mice. Vaccination groups were adjuvant only (∘), Vector (□), HIF1α Peptides (▴), or pHIF1α (▪). Error bars show SEM for each group. HIF1α peptide vaccinated mice and HIF1α DNA vaccinated mice had significantly smaller tumor burden vs. control mice as early as 24 days after implant. ****p<0.0001 vs. adjuvant only group. (D) IFN-g ELISPOT assessed T-cell responses to peptide or control stimulations. Each plotted point represents the spots per well of individual FVB/NJ mice in vaccination groups treated with adjuvant only (∘), Vector (□), HIF1α Peptides (▴), or pHIF1α (▪). Lines show Mean & SEM of responses. “¹” p<0.001 HIF1α peptides vs. No Ag response. Although HIF1α plasmid generated DTH responses, IFNγ ELISPOT are low level.

For another example, FIG. 22 CD105 peptide and plasmid vaccine immunogenicity and efficacy were determined in mice. (A) DTH responses were measured by change in ear thickness (mm) 24 hours after application of CD105 peptide mix in 50% DMSO. Plotted are responses of individual FVB/NJ mice from the different vaccination cohorts: Controls (both adjuvant only and vector groups, see Methods), CD105 Peps (peptide vaccine), and pCD105 (plasmid vaccine). Dotted line represents 0.0 mm change in ear thickness from baseline. * p<0.05, ***p<0.001 vs. controls. (B) DTH responses measured 24 hours after application of CD105 peptide mix in 50% DMSO. Plotted are responses of individual MMTV-C3(1)-Tag transgenic mice from the different vaccination cohorts as listed above. *** p<0.001 vs. controls. (C) Efficacy of vaccines to control M6 tumor growth was assessed by measuring tumor volume (mm3) over time post-implant (days) in MMTV-C3(1)-Tag transgenic mice. Vaccination groups were adjuvant only (∘), Vector (□), CD105 Peptides (▴), or pCD105 (▪). Error bars show SEM for each group. CD105 peptide vaccinated mice and CD105 DNA vaccinated mice had significantly smaller tumor burden vs. control mice as early as 24 days after implant. ****p<0.0001 vs. adjuvant only group. (D) IFN-g ELISPOT assessed T-cell responses to peptide or control stimulations. Each plotted point represents the spots per well of individual FVB/NJ mice in vaccination groups treated with adjuvant only (∘), Vector (□), CD105 Peptides (▴), or pCD105 (▪). Lines show Mean & SEM of responses. No significance found for CD105 peptide responses in any group.

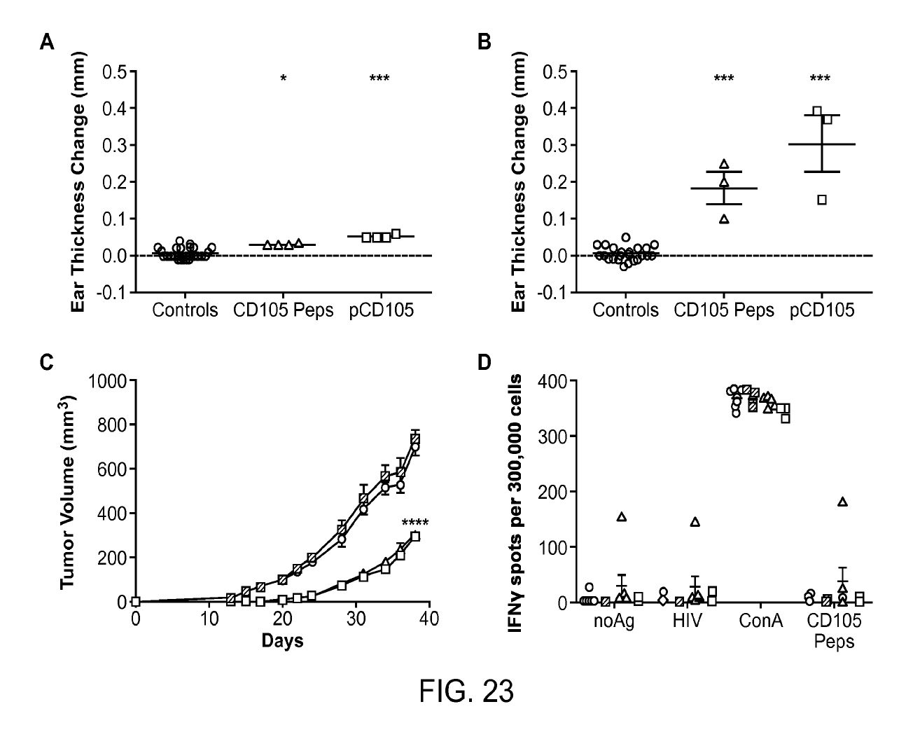

For another example, FIG. 23 CDH3 peptide and plasmid vaccine immunogenicity and efficacy were determined in mice. (A) DTH responses were measured by change in ear thickness (mm) 24 hours after application of CDH3 peptide mix in 50% DMSO. Plotted are responses of individual FVB/NJ mice from the different vaccination cohorts: Controls (see Methods), CDH3 Peps (peptide vaccine), pCDH3 (plasmid vaccine) and pUbVV-CDH3. Dotted line represents 0.0 mm change in ear thickness from baseline. * p<0.05, ** p<0.01 vs. controls. (B) DTH responses measured 24 hours after application of CDH3 peptide mix in 50% DMSO. Plotted are responses of individual FVB/N/Tg-neu transgenic mice from the different vaccination cohorts as listed above. * p<0.05, ** p<0.01 vs. controls. (C) Efficacy of vaccines to control MMC tumor growth was assessed by measuring tumor volume (mm3) over time post-implant (days) in FVB/N/Tg-neu transgenic mice. Vaccination groups were adjuvant only (∘), Vector (□), CDH3 Peptides (▴), pCDH3 (▪), or pUBVV-CDH3 (♦). Error bars show SEM for each group. Neither CDH3 peptide or DNA vaccinated mice had significantly smaller tumor burden vs. control mice. (D) IFNγ ELISPOT assessed T-cell responses to peptide or control stimulations. Each plotted point represents the spots per well of individual FVB/NJ mice in vaccination groups treated with adjuvant only (∘), CDH3 Peptides (▴), pCDH3 (▪), or pUBVV-CDH3 (4). Lines show Mean & SEM of responses. **** p<0.0001 CDH3 peptides vs. No Ag response.

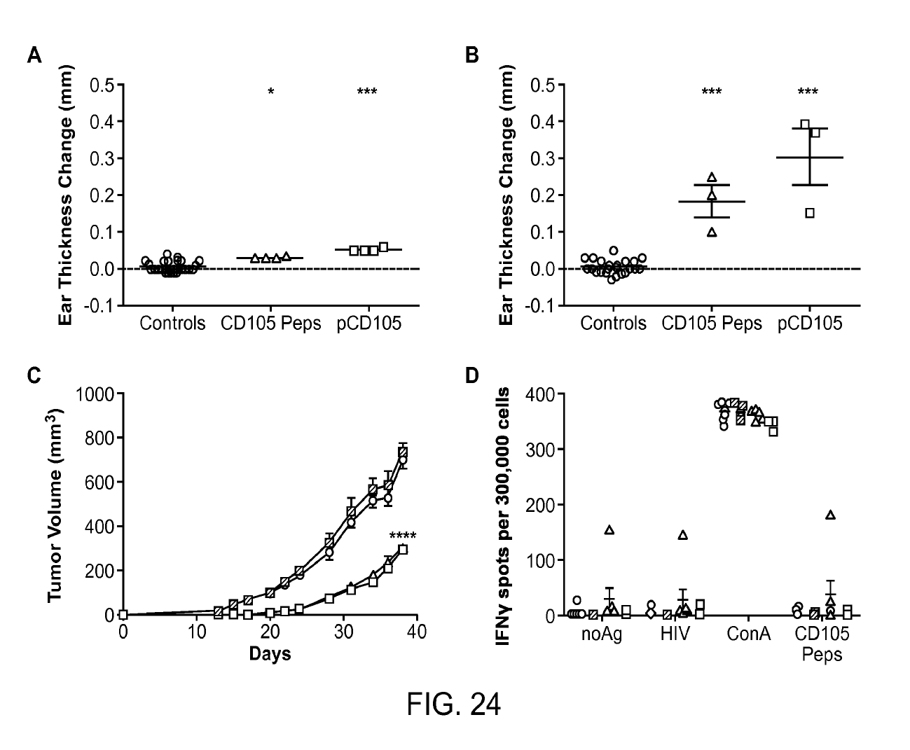

For another example, FIG. 24, SOX2 peptide and plasmid vaccine immunogenicity and efficacy were determined in mice. (A) DTH responses were measured by change in ear thickness (mm) 24 hours after application of SOX2 peptide mix in 50% DMSO. Plotted are responses of individual FVB/NJ mice from the different vaccination cohorts: Controls (see Methods), SOX2 Peps (peptide vaccine), pSOX2 (plasmid vaccine) and pUbVV-SOX2. Dotted line represents 0.0 mm change in ear thickness from baseline. No significance found versus controls. (B) DTH responses measured 24 hours after application of SOX2 peptide mix in 50% DMSO. Plotted are responses of individual FVB/N/Tg-neu transgenic mice from the different vaccination cohorts as listed above. * p<0.05 vs. controls. (C) Efficacy of vaccines to control MMC tumor growth was assessed by measuring tumor volume (mm3) over time post-implant (days) in FVB/N/Tg-neu transgenic mice. Vaccination groups were adjuvant only (∘), Vector (□), SOX2 Peptides (▴), pSOX2 (▪), or pUBVV-SOX2 (♦). Error bars show SEM for each group. *** p<0.001, ****p<0.0001 vs. adjuvant only group. (D) IFNγ ELISPOT assessed T-cell responses to peptide or control stimulations. Each plotted point represents the spots per well of individual FVB/NJ mice in vaccination groups treated with adjuvant only (∘), SOX2 Peptides (▴), pSOX2 (▪), or pUBVV-SOX2 (♦). Lines show Mean & SEM of responses. *** p<0.001 SOX2 peptides, **** p<0.0001 pSOX2, **** p<0.0001 pUbVV-SOX2 vs. No Ag response. Although SOX2 peptide and plasmid generated IFN-γ ELISPOT responses, DTH responses are low level or not significant in plasmid and peptide.

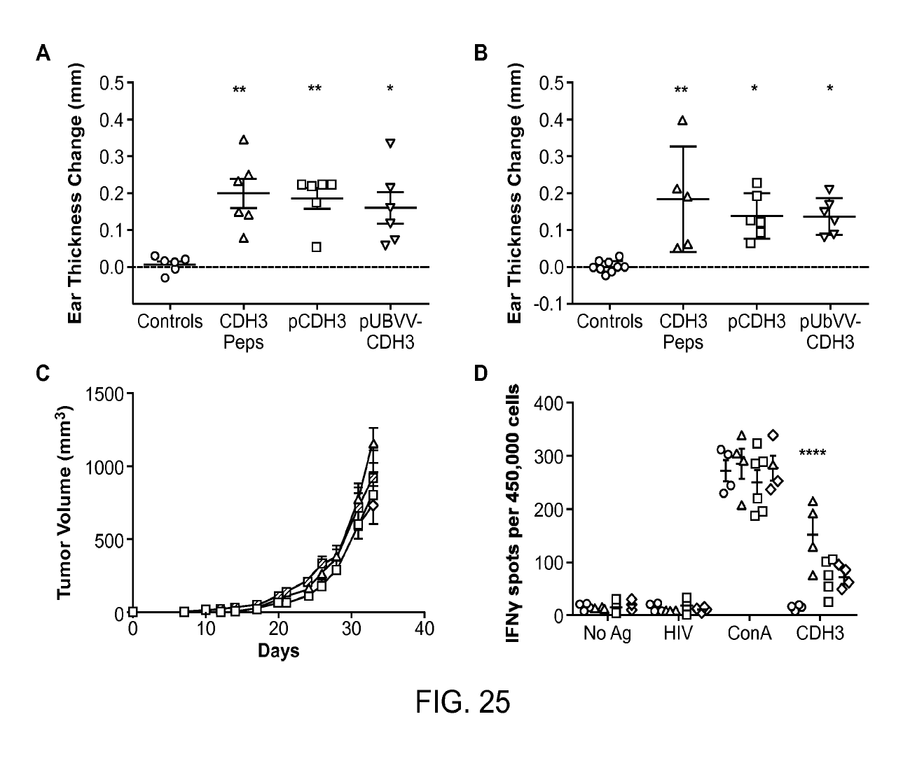

For another example, FIG. 25 MDM2 peptide and plasmid vaccine immunogenicity and efficacy were determined in mice. (A) DTH responses were measured by change in ear thickness (mm) 24 hours after application of MDM2 peptide mix in 50% DMSO. Plotted are responses of individual FVB/NJ mice from the different vaccination cohorts: Controls (see Methods), MDM2 Peps (peptide vaccine), pMDM2 (plasmid vaccine) and pUbVV-MDM2. Dotted line represents 0.0 mm change in ear thickness from baseline. *** p<0.001 vs. controls. (B) DTH responses measured 24 hours after application of MDM2 peptide mix in 50% DMSO. Plotted are responses of individual FVB/N/Tg-neu transgenic mice from the different vaccination cohorts as listed above. p<0.001 vs. controls. (C) IFNγ ELISPOT assessed T-cell responses to peptide or control stimulations. Each plotted point represents the spots per well of individual FVB/NJ mice in vaccination groups treated with adjuvant only (∘), Vector (□), MDM2 Peptides (▴), pMDM2 (▪), or pUBVV-MDM2 (♦). Lines show Mean & SEM of responses. *** p<0.001 pMDM2 vs. No Ag response.

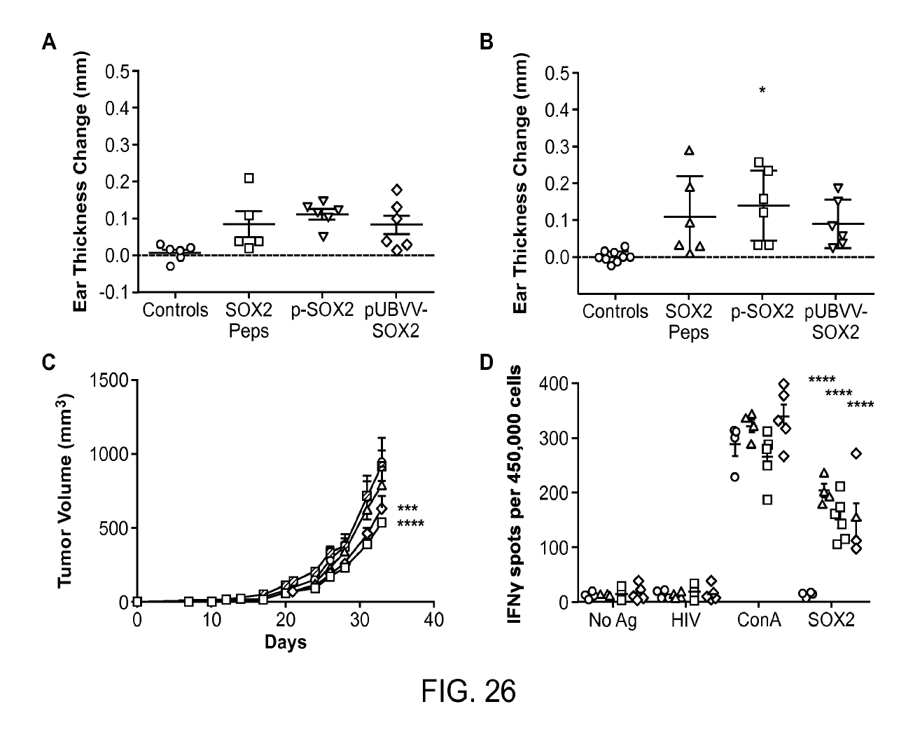

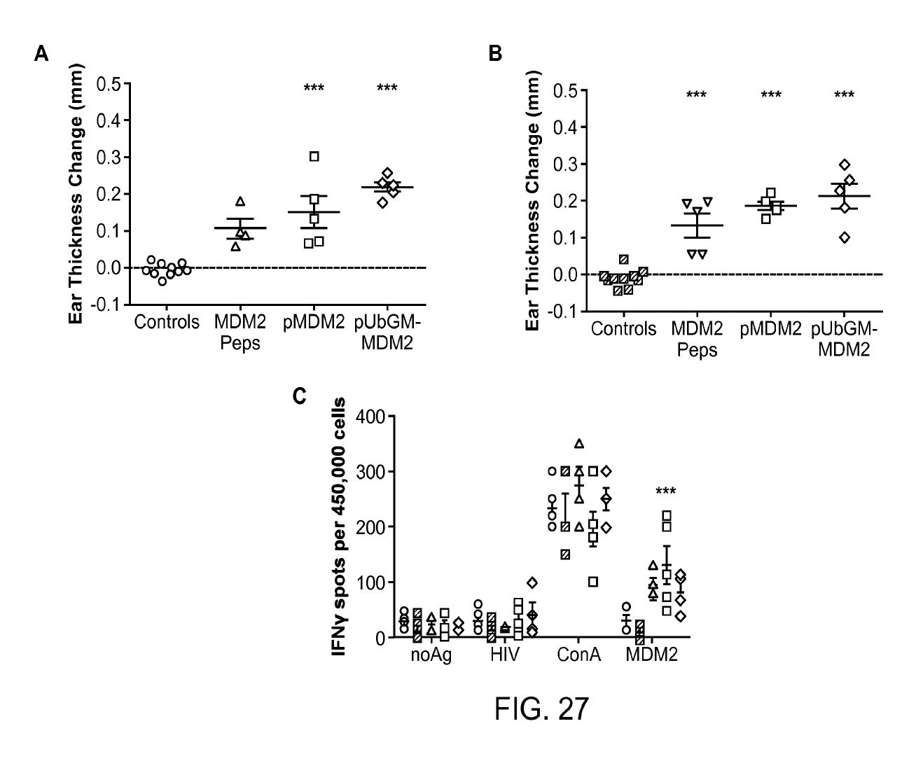

Following vaccination, the masses of mice were determined. For example, at FIG. 26 the mass of mice three months after the last vaccine. Mice (n=5) were left untreated, immunized with pUMVC3 alone, pUMVC3-hHIF1α (30-119), or pUMVC3-hCD105 (87-138), x-axis, with CFA/IFA as an adjuvant. The mass of each mouse, y-axis, (mean±SEM) was recorded three months after the last vaccine. The mass of mice was also determined ten days after the last vaccine. See FIG. 27 Mice (n=5) were left untreated, immunized with pUMVC3 alone, pUMVC3-hHIF1α (30-119), or pUMVC3-hCD105 (87-138), x-axis, with CFA/IFA as an adjuvant. The mass of each mouse, y-axis, (mean±SEM) was recorded ten days after the last vaccine.

Determination and Construction of Sequences for Short and Extended Epitopes from IGF-1R.

Two plasmids were constructed, the DNA sequences verified, and used for vaccination experiments. The short epitope plasmid, pIGF-IRshep, expressed a protein with tandemly linked MHC II epitopes corresponding to human IGF-IR. Additionally, there are four amino acids at the N-terminus (MAVP) and three amino acids at the C-terminus (AAA) that are not related to the IGF-IR sequence. The extended epitope plasmid, pIGF-IRexep, expresses a protein with two 1360. Additionally, there are four amino acids at the N-terminus (MAVP) and three amino acids at the C-terminus (AAA) that are not related to the IGF-IR sequence. The vector backbone of each plasmid is pUMVC3, which contains the CMV promoter, directing constitutive expression of the genes in mammalian cells. This vector is qualified for clinical use. The chosen epitopes in the C-terminal region of IGF-IR were assayed with synthetic peptides and demonstrated a propensity to induce greater stimulation of Th1 (IFNγ) compared to Th2 (IL-10) cells in ELISPOT assays of human PBMC samples (described in original proposal).

As T-cell immunity is required for the generation of anti-tumor antibodies, a delayed type hypersensitivity (DTH) assay to show that antigen-specific reactive T-cells were generated by pIGF-IRexep vaccination was performed. FVB mice were received three injections, at two week intervals, with either pIGF-IRexep, pUMVC3 vector, IGF-IR peptides, or adjuvant alone (plasmids and peptides were dosed at 50 ug/injection with CFA/IFA adjuvant). Two weeks after the 3rd vaccination the DTH assay was performed by vigorously rubbing either PBS or the IGF-IR peptide mix on to the mouse ears, and ear swelling was monitored for three days. The results demonstrate that significant DTH responses to IGF-IR peptides occurred in peptide-vaccinated and pIGF-IRexep-vaccinated mice compared to vector and adjuvant controls compared to ears treated with PBS (p<0.05, 4-48 hrs by one way ANOVA). Neither vector nor adjuvant controls had significant DTH reactions compared to PBS treatments.

Evaluation of the Clinical Efficacy of Short Vs. Extended IGF-1R Epitopes in TgMMTVneu Mice.

In order to directly compare the ability of the short and extended epitope plasmid vaccines to control tumor growth, a syngeneic tumor implant model was employed. Mice (TgMMTVneu) were separated into 4 vaccination groups (pIGF-IRexep, pIGF-IRshep, vector, and IGF-IR peptides) and implanted with syngeneic breast cancer cells (MMC) 7 days after the 3rd vaccination. Dosages were as stated above. The ability of MMC cells to form a tumor, and the tumor growth rate was measured. The IGF-IR peptide vaccine, the short epitope plasmid vaccine, and the extended epitope plasmid vaccine all significantly controlled tumor growth compared to the group that was vaccinated with vector alone (p<0.0001, from 14-31 days). The mice vaccinated with pIGF-IRexep had the slowest growing tumors, but they were not significantly different from tumor growth in animals vaccinated with pIGF-IRshep, p>0.05.

Determination of the Mechanism of Action of the Therapeutic Efficacy Via Blocking Studies.

In order to further delineate the role of B and T-cells in the tumor protection mediated by the pIGF-IRexep and IGF-IR peptide vaccines, critical effectors were blocked using depleting antibodies specific for T- and B-cells. Mice were depleted of lymphocyte classes with specific antibodies following vaccination. MMC tumor growth was measured after vaccination in animals depleted for T or B cells. The pIGF-IRexep vaccine was tumor protective compared to vector-vaccinated animals (p<0.01), except in the groups where B- or T-cells had been depleted. This result indicates a role for both lymphocyte classes in the protective immune response. The IGF-IR peptide vaccine was tumor protective compared to vector vaccinated animals (p<0.01), except in the group where T-cells had been depleted. Depletion of B-cells had no significant effect on tumor protection by the peptide vaccine. The extended epitope plasmid vaccine can induce tumor protective immunity through both B- and T-cells, but the short epitope peptides induce only tumor protective T-cell immunity.

1-140. (canceled)

141. A method for designing a plasmid vaccine, the method comprising:
a) determining a set of putative epitopes to induce a sub-type of an immune response, wherein the sub-type of the immune response is selected from: production of IgG antibodies, production of specific Th cells in response to the set of putative peptides, or a combination thereof;
b) ranking a plurality of putative epitopes from the set of putative epitopes by the sub-type of the immune response;
c) from the plurality of putative epitopes ranked in step (b), identifying a set of desired epitopes such that the set of desired epitopes induces a desired sub-type of an immune response in a subject; and
d) arranging the desired epitopes to provide a plasmid vaccine design.

142. The method of claim 141, wherein the set of putative epitopes comprise a set of epitopes of self-proteins of the subject.

143. The method of claim 141, wherein the set of putative epitopes contains epitopes from between about 2 and about 50 unique peptides.

144. The method of claim 141, wherein the set of putative epitopes is overexpressed in a subject with a disease compared to a subject without a disease.

145. The method of claim 141, wherein at step (a), the method further comprises identifying the set of putative epitopes by a method selected from: a literature search, a database search, a search of bio informatics mediums, an analysis of a fluid sample from a subject, an analysis of a cellular sample from a subject, an analysis of a tissue sample from a subject, or a combination thereof.

146. The method of claim 141, wherein at step (b), the method further comprises ranking the plurality of putative epitopes from the set of putative epitopes by a method selected from: a literature search, a database search, a search of bio informatics mediums, analysis of a fluid sample from a subject, analysis of a cellular sample from a subject, analysis of a tissue sample from a subject, or a combination thereof.

147. The method of claim 141, wherein at step (b), the method further comprises ranking the plurality of putative epitopes from the set of putative epitopes by identifying an adaptive immune response to the set of putative peptides in a subject.

148. The method of claim 141, wherein the sub-type of the immune response is identified by an assay selected from: an enzyme linked immunosorbant assay (ELISA), an enzyme linked immunosorbant spot (ELISPOT) assay, a delayed type hypersensitivity responses (DTH), a lymphocyte proliferation or a cytoxicity assay, or a combination thereof.

149. The method of claim 141, wherein at step (b), ranking includes ranking each epitope in the set of putative epitopes according to a parameter selected from: binding of each epitope to major histocompatibility complex (MHC) alleles, affinity of each epitope for major histocompatibility complex (MHC) alleles, or a combination thereof.

150. The method of claim 149, wherein each epitope ranked in the top two quartiles of the set of putative epitopes is identified in the set of desired epitopes.

151. The method of claim 141, wherein the sub-type of the immune response is a Type I immune response and the Type I response is determined by measuring production of interferon gamma (IFNγ), interleukin-12 (IL-12), or TNFα in the subject.

152. The method of claim 151, wherein IFNγ is measured using an assay selected from: ELISPOT assay, ELISA, rtPCR analysis of mRNA expression, immunohistochemistry, fluorescence in situ hybridization analysis (FISH), or a combination thereof.

153. The method of claim 141, wherein the sub-type of the immune response is a Type II immune response and the Type II immune response is determined by measuring production of interleukin-10 (IL-10), interleukin-4 (IL-4), interleukin-5 (IL-5), or interleukin-6 (IL-6) in the subject.

154. The method of claim 153, wherein IL-10 is measured using an assay selected from: ELISPOT assay, ELISA, rtPCR analysis of mRNA expression, immunohistochemistry, and fluorescence in situ hybridization analysis (FISH), or a combination thereof.

155. The method claim 141, wherein each epitope within the set of putative epitopes is differentiated by induction of a Type I immune response.

156. The method of claim 141, wherein each epitope within the set of putative epitopes is differentiated by induction of a Type II immune response.

157. The method of claim 141, wherein the arranging of the desired epitopes comprises separating two or more epitopes with a sequence of linker nucleic acids.

158. The method of claim 141, further comprising step (e), administering a plasmid vaccine of step (d) to the subject.

159. The method of claim 141, wherein the putative epitopes are extended epitopes.

160. The method of claim 141, wherein the putative epitopes are derived from the same peptide.

161. The method of claim 141, wherein the desired sub-type of the immune response is characterized by a ratio of Type I cytokine production to Type II cytokine production that is greater than 1.

162. The method of claim 141, wherein the desired sub-type of the immune response is characterized by a ratio of Type I cytokine production to Type II cytokine production that is less than 1.

163. The method of claim 141, further comprising step (e), producing a plasmid vaccine of step (d), wherein the plasmid vaccine comprises a set of nucleic acid sequences encoding a set of amino acids of the set of desired epitopes.


SEQUENCE LISTING (15 sequences)

SEQ ID NO: 1 - protein, 52 aa, Artificial Sequence
  Synthetic polypeptide
QNGTWPREVLLVLSVNSSVFLHLQALGIPLHLAYNSSLVTFQEPPGVNTTEL

SEQ ID NO: 2 - protein, 90 aa, Artificial Sequence
  Synthetic polypeptide
RSKESEVFYELAHQLPLPHNVSSHLDKASVMRLTISYLRVRKLLDAGDLDIEDDMKAQMNCFYLKALDGFVMVLTDDGDMIYISDNVNKY

SEQ ID NO: 3 - protein, 16 aa, Artificial Sequence
  Synthetic polypeptide
YELAHQLPLPHNVSSH

SEQ ID NO: 4 - protein, 23 aa, Artificial Sequence
  Synthetic polypeptide
MRLTISYLRVRKLLDAGDLDIED

SEQ ID NO: 5 - protein, 25 aa, Artificial Sequence
  Synthetic polypeptide
LKALDGFVMVLTDDGDMIYISDNVN

SEQ ID NO: 6 - protein, 20 aa, Artificial Sequence
  Synthetic polypeptide
VLLVLSVNSSVFLHLQALGI

SEQ ID NO: 7 - protein, 15 aa, Artificial Sequence
  Synthetic polypeptide
LHLAYNSSLVTFQEP

SEQ ID NO: 8 - protein, 23 aa, Artificial Sequence
  Synthetic polypeptide
GHKEAHILRVLPGHSAGPRTVTV

SEQ ID NO: 9 - DNA, 31 bp, Artificial Sequence
  Synthetic polynucleotide
actggaattcaccgccagcatgctgccgaga

SEQ ID NO: 10 - DNA, 30 bp, Artificial Sequence
  Synthetic polynucleotide
cagtggatccctactgcatccgctgggtgt

SEQ ID NO: 11 - DNA, 42 bp, Artificial Sequence
  Synthetic polynucleotide
actggaattcaccgccagcatgaaccacgtggacagcaccat

SEQ ID NO: 12 - DNA, 30 bp, Artificial Sequence; identical to SEQ ID NO: 10
  Synthetic polynucleotide

SEQ ID NO: 13 - DNA, 27 bp, Artificial Sequence
  Synthetic polynucleotide
gaattcaccgccagcatgctgccgaga

SEQ ID NO: 14 - DNA, 26 bp, Artificial Sequence
  Synthetic polynucleotide
ggatccctactgcatccgctgggtgt

SEQ ID NO: 15 - protein, 328 aa, Artificial Sequence
  Synthetic polypeptide
MAVPMQLSCSRQNGTWPREVLLVLSVNSSVFLHLQALGIPLHLAYNSSLVTFQEPPGVNTTELRSTGGVPVQGSKYAADRNHYRRYPRRRGPPRNYQQNTRGLNAHGAAQMQPMHRYDVSALQYNSMTSSQTYMNGSPTYSMSYSQQGTPGMALGSMGSVRSQLRSLKERNPLKIFPSKRILRRHKRDWVVAPISVPENGKGPFPQRLNQLKSNKDRDTKIFYSITGPGADSPPEGVFAVEKETRSAGETYTMKEVLFYLGQYIMTKRLYDEKQQHIVYCSNDLLGDLFGVPSFSVKEHRKIYTMIYRNLVVVNQQESSDSGTSVSRS